(19) United States
(12) Patent Application Publication
(10) Pub. No.: US 2024/0139188 A1
(43) Pub. Date: May 2, 2024

(54) Arylamide Compounds For Treatment And Prevention Of Fungal Infections

(71) Applicant: Innovation Pharmaceuticals Inc., Wakefield, MA (US)

(72) Inventors: Gustavo H. Goldman, Sao Paulo (BR); Thaila Fernanda dos Reis, Sao Paulo (BR)

(21) Appl. No.: 18/472,450

(22) Filed: Sep. 22, 2023

Related U.S. Application Data
(60) Provisional application No. 63/409,800, filed on Sep. 25, 2022.
(60) Provisional application No. 63/451,012, filed on Mar. 9, 2023.

(51) Int. Cl. A61K 31/506; A61K 38/12; A61P 31/10

(52) U.S. Cl. CPC A61K 31/506; A61K 38/12; A61P 31/10

(57) ABSTRACT

The present disclosure provides methods for treating or preventing a fungal infection with one or more arylamide compounds, or pharmaceutically acceptable salts thereof, or compositions comprising the same and an additional anti-fungal agent, and pharmaceutical compositions comprising one or more arylamide compounds and at least one additional anti-fungal agent.

### FIELD

The present disclosure is directed, in part, to methods of treating or preventing a fungal infection with one or more arylamide compounds, or pharmaceutically acceptable salts thereof, or compositions comprising the same.

### BACKGROUND

Fungal diseases occur in more than 1 billion people worldwide and are responsible for 1.5 million deaths (Bongomin et al., J. Fungi (Basel), 2017. 3. 57; and Brown et al., Science Transl. Med., 2012, 4, 165). Aspergillosis encompasses a group of heterogeneous diseases caused by Aspergillus spp. (Rudramurthy et al., J. Fungi, 2019, 5, 1-23). In immunocompetent and immunosuppressed patients, aspergillosis are characterized by noninvasive and invasive diseases, respectively (Alastruey-Izquierdo et al., Respiration, 2018, 96, 159-170; Denning et al., Eur. Respir. J., 2016, 47, 45-68; Patterson et al., Clin. Infect. Dis., 2016, 63, 433-442; Perlin et al., Lancet Infect. Dis., 2017, 17, e383-e392; and Rudramurthy et al., J. Fungi, 2019, 5, 1-23). The most lethal form of aspergillosis in recipients of both hematopoietic stem cells and solid-organ transplants is invasive pulmonary aspergillosis (IPA) and A. fumigatus is the leading cause of this disease, which comprises more than 300,000 cases worldwide and is associated with a mortality rate of up to 90% in the most susceptible populations (Almyroudis et al., Med. Mycol., 2005, 43, 247-259; Azie et al., Diagn. Microbiol. Infect. Dis., 2012, 73, 293-300; Brown et al., Science Transl. Med., 2012, 4, 165; Gonçalves et al., Mycoses. 2016, 59, 198-219; Guinea et al., Clin. Microbiol. Infect., 2010, 16, 870-877; Rudramurthy et al., J. Fungi, 2019, 5, 1-23; and Rüping et al., Drugs, 2008, 68, 1941-1962).

Azoles (itraconazole, posaconazole, voriconazole, and isavuconazole) are fungicidal drugs for A. fumigatus and are used as first line therapy against IPA while the fungistatic echinocandins, such as caspofungin (CAS), can be used as salvage therapy and have been recommended in combination therapies against emerging azole-resistant infections (Jenks et al., J. Fungi (Basel), 2018, 4, 98; Mavridou et al., Antimicrob. Agents Chemother., 2015, 59, 1738-44; and Ostrosky-Zeichner et al., Infect. Dis. Clin. North Am., 2017, 31, 475-487). Azoles inhibit the ergosterol biosynthesis pathway by directly targeting the cyp51/erg11 encoding the lanosterol 14-demethylase (Perfect, Nat. Rev. Drug Discov., 2017, 16, 603-616; and Robbins et al., Annu. Rev. Microbiol., 2017 71, 753-775). CAS acts by noncompetitively inhibiting the fungal β-1,3-glucan synthase (Fks1), required for the biosynthesis of β-1,3-glucan, and essentially blocking fungal cell wall synthesis (Perlin, Ann. N. Y. Acad. Sci., 2015, 1354, 1-11). Specifically, caspofungin affects the composition and organization of the A. fumigatus cell wall with hyphae hyperbranching, lysis of hyphal apical compartments, resulting in loss of cell wall β-1,3-glucan. In the presence of CAS, the cell wall integrity (CWI) pathway is activated, and the CWI mitogen-activated protein kinase MpkA and the phosphatase calcineurin turn on the transcription factors RlmA and CrzA, which translocate to the nucleus and regulate the activation of several stress responses and cell wall modifications, including chitin synthase gene expression resulting in overproduction of chitin (Ries et al., mBio., 2017, 8, e00705-17; and Soriani et al., Mol. Microbiol., 2008, 67, 1274-1291).

Considering the paucity of available antifungal drugs and the increasing number of azole-resistant environmental isolates, clinical azole-resistant A. fumigatus isolates are currently a crucial problem and a major threat to immunosuppressed patients (Arikan-Akdagli et al., J. Fungi, 2018, 4, 1-13; Chen et al., J. Mycol. Med., 2020, 30, 100915; Garcia-Rubio et al., Drugs, 2017, 77, 599-613; Resendiz Sharpe et al., Med. Mycol., 2018, 56(suppl_1), 83-92; Verweij et al., J. Antimicrob. Chemother., 2016, 71, 2079-2082;

Wiederhold et al., Curr. Opin. Infect. Dis., 2020, 33, 290-297; Wiederhold, Infect. Drug Resist., 2017 10, 249-259; and Bastos et al., PloS Pathog, 2021, 17, e1010073).

Because of the scarcity in antifungal agents currently in development (Hoenigl et al., Drugs, 2021, 81, 1703-1729), repurposing of currently approved drugs alone or in combination with currently used antifungal agents, presents a potential opportunity for the discovery of new antifungal agents (Nosengo, Nature, 2016, 534, 314-316; Kaul et al., Future Microbiol., 2019, 14, 829-831; Iyer et al., Nat. Ver. Microbiol., 2021, 19, 454-466). By using this strategy, several compounds have already been identified as potential new antifungal agents, and more importantly as potentiators of antifungal drugs currently in clinical use (Rhein et al., Lancet Infect. Dis., 2016, 16, 809-818; Joffe et al., Front. Microbiol., 2017, 8, 1-14; Duffy et al., Antimicrob. Agents Chemother., 2017, 61, 9; Wall et al., J. Fungi, 2019 5, 4; Revie et al., mSphere, 2020, 5, e00256-20; Iyer et al., Nat. Commun., 2020, 11, 6429; Iyer et al., Nat. Ver. Microbiol., 2021, 19, 454-466).

Brilacidin (BRI) is a non-peptidic host defense peptide/protein (HDP) mimetic that has been administered to patients or healthy volunteers in a total of 9 clinical trials, with successful demonstration of efficacy in Phase 2 human trials: i) intravenously for treatment of acute bacterial skin and skin structure infections (and also some beneficial treatment effects when administered intravenously for treatment of COVID-19); ii) by oral rinse for prevention of chemoradiation-induced oral mucositis in head and neck cancer patients; and iii) by retention enema for treatment of ulcerative proctitis or ulcerative proctosigmoiditis. An established safety and efficacy profile for brilacidin is available for these routes of administration.

Clearly, there is a high medical need for the development of safe and effective therapies that can treat or prevent fungal infections.

### SUMMARY

The present disclosure provides pharmaceutical compositions comprising a compound having the formula:

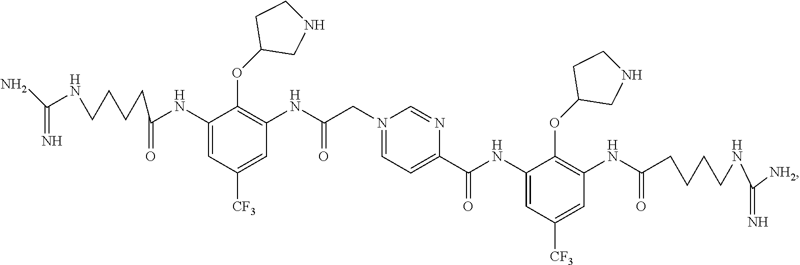

or a pharmaceutically acceptable salt thereof, and one or more other anti-fungal agents which is an azole or an echinocandin.

The present disclosure also provides methods of treating or preventing a Cryptococcus fungal infection in a mammal comprising administering to the mammal in need thereof a compound having the formula:

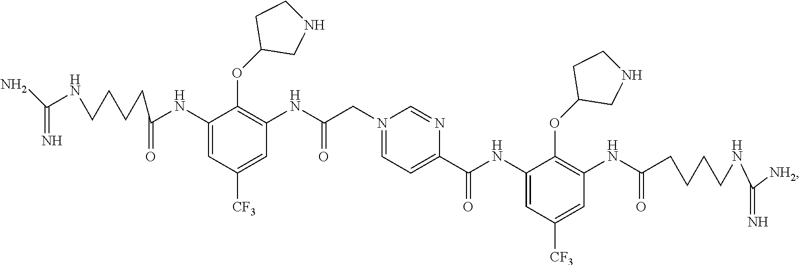

or a pharmaceutically acceptable salt thereof.

The present disclosure also provides methods of killing or inhibiting the growth of a Cryptococcus species comprising contacting the Cryptococcus species with a compound having the formula:

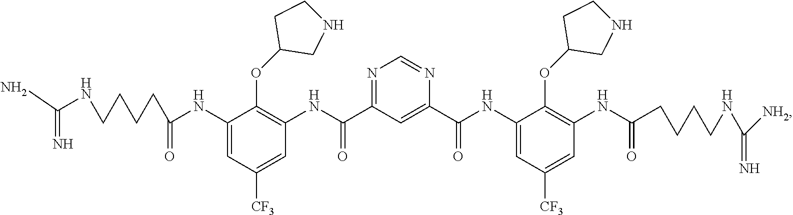 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3OC3CCNC3)ncn2)c1OC1CCNC1]

or a pharmaceutically acceptable salt thereof.

The present disclosure also provides methods of treating or preventing a fungal infection in a mammal comprising administering to the mammal in need thereof: a compound having the formula:

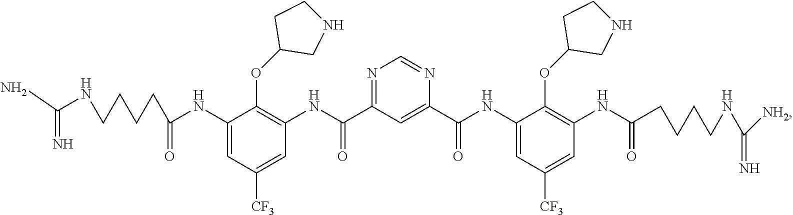 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3OC3CCNC3)ncn2)c1OC1CCNC1]

or a pharmaceutically acceptable salt thereof; and one or more other anti-fungal agents which is an azole or an echinocandin.

The present disclosure also provides methods of killing or inhibiting the growth of a fungus comprising contacting the fungus with: a compound having the formula:

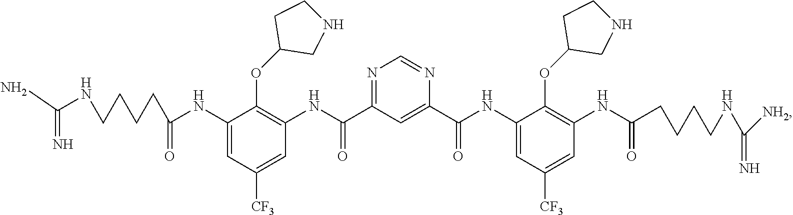 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3OC3CCNC3)ncn2)c1OC1CCNC1]

or a pharmaceutically acceptable salt thereof; and one or more other anti-fungal agents which is an azole or an echinocandin.

The present disclosure provides a compound having the formula:

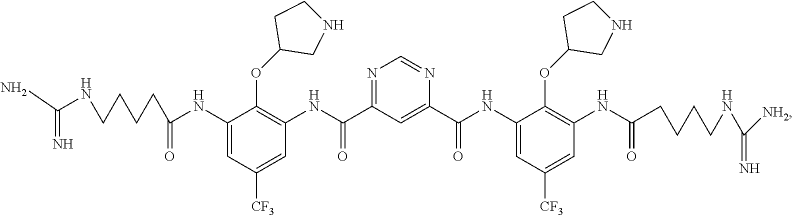 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3OC3CCNC3)ncn2)c1OC1CCNC1]

or a pharmaceutically acceptable salt thereof, for use in the treatment of a Cryptococcus fungal infection.

The present disclosure provides pharmaceutical compositions comprising a compound having the formula:

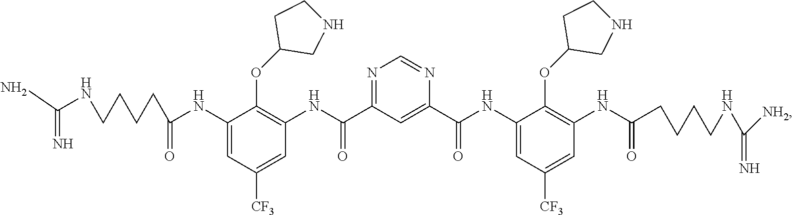 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3OC3CCNC3)ncn2)c1OC1CCNC1]

or a pharmaceutically acceptable salt thereof, and one or more other anti-fungal agents which is an azole or an echinocandin for use in the treatment of a fungal infection.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows screening of repurposing chemical libraries identified several compounds that enhance or synergize caspofungin activity; Panel A shows a heat map of % of activity using Alamar blue; the % of activity is based on A. fumigatus grown in the absence or presence of a specific compound (minimal medium (MM)+CAS 0.2 μg/ml or enhancers or synergizers 20 μM alone, or a combination of enhancer or synergizers from 0.6 to 20 μM) divided by the control (MM), both grown for 48 hours at 37° C.; hierarchical clustering was performed in Multiple Experiment Viewer (MeV) (word wide web at “mev.tm4.org/”), using Pearson correlation with complete linkage clustering; heat map scale and gene identities are shown; Panel B shows chemical structures of the synergizers.

FIG. 2 shows that BRI can convert CAS into a fungicidal drug; Panel A shows A. fumigatus conidia were incubated for 48 hours at 37° C. with different combinations of BRI+CAS and BRI+VOR; after this period, non-germinated conidia were plated on MM and colony forming units (CFUs) were assessed; the results are expressed as the % of viable conidia with respect to initial inoculum and are the average of three repetitions±standard deviation (p<0.0001); Panel B shows BRI+CAS disrupts the A. fumigatus membrane potential; A. fumigatus was grown for 16 hours at 37° C. and exposed to CAS 0.125 μg/ml, BRI 1 μM, or CAS 0.125 μg/ml+BRI 1 μM for 30 minutes and 3 μg/ml DIBAC₄(3); germlings were previously transferred to MM without glucose (non-carbon source, NCS) for 4 hours or not (MM) before adding CAS, BRI, or CAS+BRI for 30 minutes and subsequently DIBAC₄(3); the results are expressed as the % of fluorescent cells and are the average of three repetitions of 50 germlings each±standard deviation; Panel C shows metabolic activity expressed by XTT of A. fumigatus biofilm formation in the presence of VOR or CAS alone and combinations of BRI+VOR and BRI+CAS; biofilm was formed for 24 hours of incubation at 37° C. and after this period 50 μL of fresh MM containing CAS, VOR, or a combination of VOR+CAS or CAS+BRI were added to the biofilm to reach the final concentration as indicated and incubated for further 12 hours at 37° C.; untreated biofilm was used as a positive control; the XTT assays were performed in six replicates and the results are expressed as the average±standard deviation (*, p<0.05, **, p<0.01, ***, p<0.001, and **** p<0.0001); Panel D shows microscopic images of A. fumigatus after 48 hours exposure to BRI 20 μM, CAS 0.5 μg/ml, BRI 20 μM+CAS 0.5 μg/ml, VOR 0.25 μg/ml, and BRI 20 μM+VOR 0.25 μg/ml; bars, 20 μm; Panel E and Panel F show the Fractional Inhibitory Concentration (FIC) index for BRI+CAS and BRI+VOR, respectively.

FIG. 3 shows calcineurin and the MAPK MpkA are important for the BRI+CAS synergism; Panel A shows metabolic activity expressed by Alamar blue of A. fumigatus grown for 48 hours in the absence or presence of BRI 20 μM or BRI 20 μM+FRAX486 5 to 80 μM (*, p<0.05); Panel B shows metabolic activity expressed by Alamar blue of A. fumigatus grown for 48 hours in the absence or presence of BRI 20 μM or BRI 20 μM+PP121 5 to 80 μM (*, p<0.05 and **, p<0.01); Panel C shows growth of the wild-type, ΔcalA, ΔcalA::calA⁺, ΔmpkA, and ΔmpkA::mpkA⁺ on MM for 5 days at 37° C.; Panel D shows metabolic activity expressed by Alamar blue of A. fumigatus wild-type, ΔcalA, and ΔmpkA grown for 48 hours in the absence or presence of BRI 20 μM (*, p<0.05 and **, p<0.01); Panel E shows metabolic activity expressed by Alamar blue of A. fumigatus grown for 48 hours in the absence or presence of BRI 20 μM or BRI 20 μM+cyclosporin 25 to 200 μg/ml (*, p<0.05); Panel F shows metabolic activity expressed by Alamar blue of A. fumigatus grown for 48 hours in the absence or presence of BRI 20 μM or BRI 20 μM+Chelerenthrine 0.78 to 6.25 μg/ml (*, p<0.05); Panel G shows metabolic activity expressed by Alamar blue of A. fumigatus grown for 48 hours in the absence or presence of BRI 20 μM or BRI 20 μM+Calphostin C 6.25 to 50 μg/ml (*, p<0.05 and **, p<0.01); all the results with Alamar blue are the average of three repetitions±standard deviation. Panel H shows CrzA:GFP translocation to the nucleus when germlings were exposed to BRI 5 μM, CAS 0.07 μg/ml or BRI 5 μM+CAS 0.07 μg/ml. Conidia were germinated in YG medium and grown for 13 hours and then exposed or not to BRI, CAS, and BRI+CAS for 30 minutes. The results are the average of two repetitions with 30 germlings each±standard deviation.

FIG. 4 shows BRI can synergize CAS in other human fungal pathogens; Panel A shows metabolic activity expressed by XTT of C. neoformans grown for 48 hours in the absence or presence of CAS 0 to 32 μg/ml or BRI 0.625 μM+CAS 0 to 32 μg/ml; percentage of survival expressed as colony forming units/ml of C. neoformans cells grown for 48 hours in the absence or presence of CAS 0 to 16 μg/ml or BRI 0.625 μM+CAS 0 to 16 μg/ml; Panel B shows metabolic activity expressed by XTT of C. albicans grown for 48 hours in the absence or presence of CAS 0 to 1 μg/ml or BRI 20 μM+CAS 0 to 1 μg/ml; percentage of survival expressed as colony forming units/ml of C. albicans cells grown for 48 hours in the absence or presence of CAS 0 to 1 μg/ml or BRI 20 μM+CAS 0 to 1 μg/ml; Panel C shows metabolic activity expressed by XTT of C. albicans CAS-resistant strains grown for 48 hours in the absence or presence of CAS 0.5 μg/ml, BRI 5 to 20 μM, or BRI 5 to 20 μM+CAS 0.5 μg/ml; Panel D shows metabolic activity expressed by XTT of C. auris grown for 48 hours in the absence or presence of CAS 0.125 μg/ml, BRI 10 μM or BRI 10 μM+CAS 0.125 μg/ml; Panel E shows metabolic activity expressed by XTT of C. auris 467/2015 strain grown for 48 hours in the absence or presence of CAS 0 to 1 μg/ml or BRI 10 μM+CAS 0 to 1 μg/ml; and percentage of survival expressed as colony forming units/ml of C. auris 467/2015 strain grown for 48 hours in the absence or presence of CAS (or 1 μg/ml or BRI 10 μM+CAS 0 to 1 μg/ml. (*, p<0.05, **, p<0.01, ***, p<0.001, and ****, p<0.0001); all the results are the average of three repetitions±standard deviation.

FIG. 5 shows the combination of BRI+CAS is not toxic to human cells and can significantly decrease the A. fumigatus fungal burden in a chemotherapeutic murine model; Panel A shows A549 lung cells grown in the absence or presence of different concentrations of BRI and CAS; Panel A shows A549 lung cells grown in the absence or presence of different concentrations of BRI and CAS, and positive control is DMSO 10%; percentage of cell viability is expressed as the absorbance value of experiment well/absorbance value of control well×100; all the results are the average of three repetitions±standard deviation; Panel B shows A549 lung cells were infected with A. fumigatus conidia in the absence or presence of different concentrations of BRI and CAS; percentage of cell viability is expressed as the absorbance value of experiment well/absorbance value of control well×100; all the results are the average of three repetitions±standard deviation; Panel C shows fungal burden was determined 72 hours post-infection by real-time qPCR based on 18S rRNA gene of A. fumigatus and an intronic region of the mouse GAPDH gene; fungal and mouse DNA quantities were obtained from the Ct values from an appropriate standard curve; fungal burden was determined through the ratio between ng of fungal DNA and mg of mouse DNA; the results are the means (±standard deviation) of five lungs for each treatment; statistical analysis was performed by using one-way ANOVA followed by Tukey's multiple comparison test.

FIG. 6 shows that BRI acts in A. fumigatus by affecting the cell wall integrity pathway; Panel A shows caspofungin displays a fungistatic activity against the wild-type, ΔmpkA, and ΔcalA strains, when conidia (2×10³) were grown for 24 hours in 200 μl liquid minimal medium (MM) supplemented or not with caspofungin 0.2 μg/ml for 24 hours at 37° C., then plated on solid MM and incubated for 48 hours at 37° C.; Panel B and Panel C show the Fractional Inhibitory Concentration (FIC) index for BRI+CAS in the ΔmpkA and ΔcalA mutant strains, respectively; Panel D shows chitin synthase mRNA accumulation in A. fumigatus wild-type, ΔmpkA and ΔcalA strains, when the A. fumigatus strains were grown for 24 hours at 37° C. and exposed or not to different concentrations of CAS and/or BRI for 1 hour; results are the average of three repetitions; Panel E shows exposure of chitin and as measured by Calcofluor White (CFW) staining; results are the average of 8 repetitions±standard deviation.

### DESCRIPTION OF EMBODIMENTS

Aspergillus fumigatus (A. fumigatus) is the main etiological agent of a group of heterogeneous diseases called aspergillosis of which the most lethal form is the invasive pulmonary aspergillosis (IPA). Fungicidal azoles and amphotericin are the first line defense against A. fumigatus, but fungistatic echinocandins, such as caspofungin (CAS), can be used as salvage therapy for IPA. Here, repurposing libraries were screened and several compounds that can potentiate CAS activity against A. fumigatus were identified, among them the host defense peptide mimetic, brilacidin (BRI). BRI converts CAS into a fungicidal drug and potentiates voriconazole (VOR) against A. fumigatus. BRI increases the ability of both CAS and VOR to control A. fumigatus biofilm growth. BRI depolarizes the A. fumigatus cell membrane leading to disruption of membrane potential. By using a combination of protein kinase inhibitors and screening of a catalytic subunit null mutant library, the mitogen activated protein kinase (MAPK) MpkA and the phosphatase calcineurin were identified as mediators of the synergistic action of BRI. These results suggest the most likely BRI mechanism of action for CAS potentiation is the inhibition of A. fumigatus cell wall integrity (CWI) pathway. BRI synergizes with CAS against Candida albicans (C. albicans), Candida auris (C. auris), and Cryptococcus neoformans (C. neoformans). Interestingly, BRI overcomes the CAS-acquired resistance in both A. fumigatus and C. albicans and the CAS-intrinsic resistance in C. neoformans. Cell toxicity assays and fungal burden studies in an immunosuppressed murine model of IPA showed that BRI combined with CAS is not toxic to the cells and significantly clears A. fumigatus lung infection, respectively. These results indicate that combinations of BRI and antifungal drugs in clinical use are likely to improve the treatment outcome of IPA and other fungal infections.

Unless defined otherwise, all technical and scientific terms have the same meaning as is commonly understood by one of ordinary skill in the art to which the embodiments disclosed belongs.

As used herein, the terms “a” or “an” means “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by +10% and remain within the scope of the disclosed embodiments.

As used herein, the term “acylamino” refers to an amino group substituted by an acyl group (e.g., —O—C(═O)—H or —O—C(═O)-alkyl). An example of an acylamino is —NHC(═O)H or —NHC(—O)CH₃. The term “lower acylamino” refers to an amino group substituted by a lower acyl group (e.g., —O—C(═O)—H or —O—C(═O)-C₁₋₆ alkyl). An example of a lower acylamino is —NHC(═O)H or —NHC(—O)CH₃.

As used herein, the term “alkyl” refers to a saturated hydrocarbon group which is straight-chained or branched. An alkyl group can contain from 1 to 20, from 2 to 20, from 1 to 12, from 1 to 10, from 1 to 8, from 1 to 6, from 1 to 4, or from 1 to 3 carbon atoms. Examples of alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl), hexyl, isohexyl, heptyl, 4,4-dimethylpentyl, octyl, nonyl, decyl, undecyl, 2,2,4-trimethylpentyl, and dodecyl, and the like.

As used herein, the term “alkenyl” refers to a straight or branched chain radical of 2 to 20 carbon atoms, unless the chain length is limited thereto, including, but not limited to, ethenyl, 1-propenyl, 2-propenyl, 2-methyl-1-propenyl, 1-butenyl, 2-butenyl, cyclohexenyl, and the like. In some embodiments, the alkenyl chain is from 2 to 10 carbon atoms in length, from 2 to 8 carbon atoms in length, or from 2 to 4 carbon atoms in length.

As used herein, the term “alkoxy” refers to a straight or branched chain radical of 1 to 20 carbon atoms, unless the chain length is limited thereto, bonded to an oxygen atom, including, but not limited to, methoxy, ethoxy, n-propoxy, isopropoxy, and the like. In some embodiments, the alkoxy chain is from 1 to 10 carbon atoms in length, from 1 to 8 carbon atoms in length, or from 1 to 6 carbon atoms in length.

As used herein, the term “alkylamino” refers to an amino group substituted by an alkyl group. An example of an alkylamino is —NHCH₂CH₃.

As used herein, the term “alkynyl” refers to a straight or branched chain radical of 2 to 20 carbon atoms, unless the chain length is limited thereto, wherein there is at least one triple bond between two of the carbon atoms in the chain, including, but not limited to, acetylene, 1-propylene, 2-propylene, and the like. In some embodiments, the alkynyl chain is from 2 to 10 carbon atoms in length, from 2 to 8 carbon atoms in length, or from 2 to 4 carbon atoms in length.

As used herein, the term “alkylene” or “alkylenyl” refers to a divalent alkyl linking group. An example of an alkylene (or alkylenyl) is methylene or methylenyl (—CH₂—).

As used herein, the term “alkylamino” refers to an amino group which is substituted with one alkyl group having from 1 to 6 carbon atoms. The term “dialkylamino” refers to an amino group which is substituted with two alkyl groups, each having from 1 to 6 carbon atoms.

As used herein, the term “alkylthio” refers to a thio group which is substituted with one alkyl group having from 1 to 6 carbon atoms.

As used herein, the term “amidino” refers to —C(═NH)NH₂.

As used herein, the term “aminoalkyl” refers to an alkyl group substituted by an amino group. An example of an aminoalkyl is —CH₂CH₂NH₂.

As used herein, the term “aminosulfonyl” refers to —S(═O)₂NH₂.

As used herein, the term “aminoalkoxy” refers to an alkoxy group substituted by an amino group. An example of an aminoalkoxy is —OCH₂CH₂NH₂.

As used herein, the term “aminoalkylthio” refers to an alkylthio group substituted by an amino group. An example of an aminoalkylthio is —SCH₂CH₂NH₂.

As used herein, the term “animal” includes, but is not limited to, humans and non-human vertebrates such as wild, domestic and farm animals.

As used herein, the term “aryl” refers to monocyclic or bicyclic aromatic groups containing from 6 to 12 carbons in the ring portion or from 6 to 10 carbons in the ring portion, such as the carbocyclic groups phenyl, naphthyl or tetrahydronaphthyl. An aryl can represent carbocyclic aryl groups, such as phenyl, naphthyl or tetrahydronaphthyl, as well as heterocyclic aryl (“heteroaryl”) groups, such as pyridyl, pyrimidinyl, pyridazinyl, furyl, and pyranyl.

As used herein, the term “arylamino” refers to an amino group substituted by an aryl group. An example of an alkylamino is —NH(phenyl).

As used herein, the term “arylene” refers to an aryl linking group, for example, an aryl group that links one group to another group in a molecule.

As used herein, the term “carbamoyl” refers to —C(═O)—NH₂.

As used herein, the term “chemically nonequivalent termini” refers to a functional group such as an ester, amide, sufonamide, or N-hydroxyoxime that, when reversing the orientation of the functional group (for example, —(C═O)O—) produces different chemical entities (for example, —R¹C(═O)OR²— versus —R¹OC(═O)R²—).

As used herein, the term, “compound” refers to all stereoisomers, tautomers, and solvates of the compounds described herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, un-recited elements or method steps.

As used herein, the term “cyano” refers to —CN.

As used herein, the term “cycloalkyl” refers to non-aromatic cyclic hydrocarbons including cyclized alkyl, alkenyl, and alkynyl groups that contain up to 20 ring-forming carbon atoms. Cycloalkyl groups can include mono- or polycyclic ring systems such as fused ring systems, bridged ring systems, and spiro ring systems. In some embodiments, polycyclic ring systems include 2, 3, or 4 fused rings. A cycloalkyl group can contain from 3 to about 15, from 3 to 10, from 3 to 8, from 3 to 6, from 4 to 6, from 3 to 5, or from 5 to 6 ring-forming carbon atoms. Ring-forming carbon atoms of a cycloalkyl group can be optionally substituted by oxo or sulfido. Examples of cycloalkyl groups include, but are not limited to, cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (having a bond in common with) to the cycloalkyl ring, for example, benzo or thienyl derivatives of pentane, pentene, hexane, and the like (e.g., 2,3-dihydro-1H-indene-1-yl, or 1H-inden-2(3H)-one-1-yl).

As used herein, the term “dialkylamino” refers to an amino group substituted by two alkyl groups.

As used herein, the term “diazamino” refers to —N(NH₂)₂.

As used herein, the term “guanidino” refers to —NH(═NH)NH₂.

As used herein, the term “halo” refers to halogen groups including, but not limited to fluoro, chloro, bromo, and iodo.

As used herein, the term “haloalkyl” refers to an alkyl group having one or more halogen substituents. Examples of haloalkyl groups include, but are not limited to, CF₃, C₂F₅, CHF₂, CCl₃, CHCl₂, C₂Cl₅, CH₂CF₃, and the like.

As used herein, the term “heteroaryl” refers to an aromatic heterocycle having up to 20 ring-forming atoms and having at least one heteroatom ring member (ring-forming atom) such as sulfur, oxygen, or nitrogen. In some embodiments, the heteroaryl group has at least one or more heteroatom ring-forming atoms, each of which are, independently, sulfur, oxygen, or nitrogen. In some embodiments, the heteroaryl group has from 1 to about 20 carbon atoms, from 1 to 5, from 1 to 4, from 1 to 3, or from 1 to 2, carbon atoms as ring-forming atoms. In some embodiments, the heteroaryl group contains 3 to 14, 3 to 7, or 5 to 6 ring-forming atoms. In some embodiments, the heteroaryl group has 1 to 4, 1 to 3, or 1 to 2 heteroatoms. Heteroaryl groups include monocyclic and polycyclic (e.g., having 2, 3 or 4 fused rings) systems. Examples of heteroaryl groups include, but are not limited to, acridinyl, benzimidazolyl, benzofuryl, benzothienyl, benzoxazolyl, benzthiazolyl, carbazolyl, furazanyl, furyl, imidazolyl, indazolyl, indolyl (such as indol-3-yl), indolinyl, indolizinyl, isobenzofuranyl, isoindolyl, isoquinolyl, isothiazolyl, isoxazolyl, naphthyridinyl, oxazolyl, perimidinyl, phenanthridinyl, phenanthrolinyl, phenazinyl, phenothiazinyl, phenoxazinyl, phthalazinyl, purinyl, pyranyl, pyrazinyl, pyrazolyl, pyridazinyl, pyridyl, pyrimidinyl, 2H-pyrrolyl, pyrrolyl, pyrryl, quinolyl, quinazolinyl, 4H-quinolizinyl, tetrazolyl, 1,2,4-thiadiazolyl, thianthrenyl, thiazolyl, thienyl, triavinyl, triazolyl, xanthenyl, and the like. Suitable heteroaryl groups also include 1,2,3-triazole, 1,2,4-triazole, 5-amino-1,2,4-triazole, imidazole, oxazole, isoxazole, 1,2,3-oxadiazole, 1,2,4-oxadiazole, 3-amino-1,2,4-oxadiazole, 1,2,5-oxadiazole, 1,3,4-oxadiazole, pyridine, and 2-aminopyridine.

As used herein, the term “heteroarylamino” refers to an amino group substituted by a heteroaryl group. An example of an alkylamino is —NH—(2-pyridyl).

As used herein, the term “heteroarylene” refers to a heteroaryl linking group, such as, a heteroaryl group that links one group to another group in a molecule.

As used herein, the term “heterocycle” or “heterocyclic ring” represents a stable 5- to 7-membered mono- or bicyclic or stable 7- to 10-membered bicyclic heterocyclic ring system any ring of which may be saturated or unsaturated, and which consists of carbon atoms and from one to three heteroatoms selected from N, O, and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. Such rings may contain one oxygen or sulfur, one to three nitrogen atoms, or one oxygen or sulfur combined with one or two nitrogen atoms. The heterocyclic ring may be attached at any heteroatom or carbon atom which results in the creation of a stable structure. Examples of such heterocyclic groups include, but are not limited to, piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyridavinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, and oxadiazolyl. Morpholino is the same as morpholinyl.

As used herein, the term “heterocycloalkyl” refers to non-aromatic heterocycles having up to 20 ring-forming atoms including cyclized alkyl, alkenyl, and alkynyl groups, where one or more of the ring-forming carbon atoms is replaced by a heteroatom such as an O, N, or S atom. Hetercycloalkyl groups can be mono or polycyclic (e.g., fused, bridged, or spiro systems). In some embodiments, the heterocycloalkyl group has from 1 to about 20 carbon atoms, or 3 to about 20 carbon atoms. In some embodiments, the heterocycloalkyl group contains 3 to 14, 3 to 7, or 5 to 6 ring-forming atoms. In some embodiments, the heterocycloalkyl group has 1 to 4, 1 to 3, or 1 to 2 heteroatoms. In some embodiments, the heterocycloalkyl group contains 0 to 3 double bonds. In some embodiments, the heterocycloalkyl group contains 0 to 2 triple bonds. Examples of heterocycloalkyl groups include, but are not limited to, morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, pyrrolidin-2-one-3-yl, and the like. In addition, ring-forming carbon atoms and heteroatoms of a heterocycloalkyl group can be optionally substituted by oxo or sulfido. For example, a ring-forming S atom can be substituted by 1 or 2 oxo (form a S(O) or S(O)₂). For another example, a ring-forming C atom can be substituted by oxo (form carbonyl). Also included in the definition of heterocycloalkyl are moieties that have one or more aromatic rings fused (having a bond in common with) to the nonaromatic heterocyclic ring including, but not limited to, pyridinyl, thiophenyl, phthalimidyl, naphthalimidyl, and benzo derivatives of heterocycles such as indolene, isoindolene, 4,5,6,7-tetrahydrothieno[2,3-c]pyridine-5-yl, 5,6-dihydrothieno[2,3-c]pyridin-7(4H)-one-5-yl, isoindolin-1-one-3-yl, and 3,4-dihydroisoquinolin-1(2H)-one-3yl groups. Ring-forming carbon atoms and heteroatoms of the heterocycloalkyl group can be optionally substituted by oxo or sulfido.

As used herein, the term “hydroxyalkyl” or “hydroxylalkyl” refers to an alkyl group substituted by a hydroxyl group. Examples of a hydroxylalkyl include, but are not limited to, —CH₂OH and —CH₂CH₂OH.

As used herein, the terms “individual” or “patient” or “subject” used interchangeably, refers to any animal, including mammals, such as mice, rats, other rodents, rabbits, dogs, cats, swine, cattle, sheep, horses, or primates, such as humans.

As used herein, the term “inhibiting the growth” means reducing by any measurable amount the growth of one or more fungi. In some embodiments, the inhibition of growth may result in cell death of the fungi.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalant.

As used herein, the term “n-membered”, where n is an integer, typically describes the number of ring-forming atoms in a moiety, where the number of ring-forming atoms is n. For example, pyridine is an example of a 6-membered heteroaryl ring and thiophene is an example of a 5-membered heteroaryl ring.

As used herein, the phrase “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent group, provided that the normal valency of the designated atom or moiety is not exceeded, and that the substitution results in a stable compound. For example, if a methyl group is optionally substituted, then 3 hydrogen atoms on the carbon atom can be replaced with substituent groups.

As used herein, the phrase “pharmaceutically acceptable” refers to those compounds, materials, compositions, and/or dosage forms which are, within the scope of sound medical judgment, suitable for use in contact with tissues of humans and animals.

As used herein, the term “semicarbazone” refers to ═NNHC(═O)NH₂.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the fungal infection being treated or the biological effect desired. As such, the therapeutic effect can be a decrease in the severity of symptoms associated with the fungal infection and/or inhibition (partial or complete) of progression of the fungal infection, or improved treatment, healing, prevention or elimination of a fungal infection, or side-effects. The amount needed to elicit the therapeutic response can be determined based on the age, health, size and sex of the subject. Optimal amounts can also be determined based on monitoring of the subject's response to treatment.

As used herein, the terms “treat,” “treated,” or “treating” mean both therapeutic treatment and prophylactic or preventative measures wherein the object is to prevent or slow down (lessen) a fungal infection, or obtain beneficial or desired clinical results. For purposes herein, beneficial or desired clinical results include, but are not limited to, alleviation of symptoms; diminishment of extent of fungal infection; stabilized (i.e., not worsening) state of fungal infection; delay in onset or slowing of fungal infection or fungal infection progression; amelioration of the fungal infection state or remission (whether partial or total), whether detectable or undetectable; an amelioration of at least one measurable physical parameter, not necessarily discernible by the patient; or enhancement or improvement of fungal infection. Treatment includes eliciting a clinically significant response without excessive levels of side effects. Treatment also includes prolonging survival as compared to expected survival if not receiving treatment.

As used herein, the term “ureido” refers to —NHC(═O)—NH₂.

At various places in the present specification, substituents of compounds described herein are disclosed in groups or in ranges. It is specifically intended that the subject matter include each and every individual subcombination of the members of such groups and ranges. For example, the term “C16 alkyl” is specifically intended to individually disclose methyl, ethyl, C₃ alkyl, C₄ alkyl, C₅ alkyl, and C₆ alkyl.

For compounds in which a variable appears more than once, each variable can be a different moiety selected from the Markush group defining the variable. For example, where a structure is described having two R groups that are simultaneously present on the same compound, the two R groups can represent different moieties selected from the Markush groups defined for R. In another example, when an optionally multiple substituent is designated in the form:

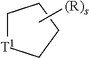

then it is understood that substituent R can occur s number of times on the ring, and R can be a different moiety at each occurrence. Further, in the above example, where the variable T¹ is defined to include hydrogens, such as when T¹ is CH₂, NH, etc., any floating substituent such as R in the above example, can replace a hydrogen of the T¹ variable as well as a hydrogen in any other non-variable component of the ring.

It is further appreciated that certain features described herein, which are, for clarity, described in the context of separate embodiments, can also be provided in combination in a single embodiment. Conversely, various features described herein which are, for brevity, described in the context of a single embodiment, can also be provided separately or in any suitable subcombination.

In some instances, the compounds described herein can be asymmetric (e.g., having one or more stereocenters). All stereoisomers, such as enantiomers and diastereomers, are intended to be included within the scope of the compounds described herein unless otherwise indicated. Compounds described herein that contain asymmetrically substituted carbon atoms can be isolated in optically active or racemic forms. Methods of preparation of optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated herein. Cis and trans geometric isomers of the compounds described herein are also included within the scope of the compounds described herein and can be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

Resolution of racemic mixtures of compounds can be carried out by any of numerous methods known in the art, including, for example, fractional recrystallization using a chiral resolving acid which is an optically active, salt-forming organic acid. Suitable resolving agents for fractional recrystallization methods include, but are not limited to, optically active acids, such as the D and L forms of tartaric acid, diacetyltartaric acid, dibenzoyltartaric acid, mandelic acid, malic acid, lactic acid, and the various optically active camphorsulfonic acids such as B-camphorsulfonic acid. Other resolving agents suitable for fractional crystallization methods include, but are not limited to, stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like. Resolution of racemic mixtures can also be carried out by elution on a column packed with an optically active resolving agent (e.g., dinitrobenzoylphenylglycine). Suitable elution solvent compositions can be determined by one skilled in the art.

Compounds described herein may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2H- isoindole, and 1H- and 2H-pyrazole. Tautomeric forms can be in equilibrium or sterically locked into one form by appropriate substitution.

Compounds described herein can also include hydrates and solvates, as well as anhydrous and non-solvated forms.

All compounds and pharmaceutically acceptable salts thereof can be prepared or be present together with other substances such as water and solvents (e.g., hydrates and solvates) or can be isolated.

Compounds described herein can also include all isotopes of atoms occurring in the intermediates or final compounds. Isotopes include those atoms having the same atomic number but different mass numbers. For example, isotopes of hydrogen include tritium and deuterium.

In some embodiments, the compounds described herein, or salts thereof, are substantially isolated. Partial separation can include, for example, a composition enriched in the compound described herein. Substantial separation can include compositions containing at least about 50%, at least about 60%, at least about 70%, at least about 80%, at least about 90%, at least about 95%, at least about 97%, or at least about 99% by weight of the compound described herein, or salt thereof. Methods for isolating compounds and their salts are routine in the art.

Compounds described herein are intended to include compounds with stable structures. As used herein, the phrases “stable compound” and “stable structure” refer to a compound that is sufficiently robust to survive isolation to a useful degree of purity from a reaction mixture, and formulation into an efficacious therapeutic agent.

The present disclosure also includes quaternary ammonium salts of the compounds described herein, where the compounds have one or more tertiary amine moiety. As used herein, the phrase “quaternary ammonium salts” refers to derivatives of the disclosed compounds with one or more tertiary amine moieties wherein at least one of the tertiary amine moieties in the parent compound is modified by converting the tertiary amine moiety to a quaternary ammonium cation via alkylation (and the cations are balanced by anions such as Cl⁻, CH₃COO⁻, and CF₃COO⁻), for example methylation or ethylation.

When any variable occurs more than one time in any constituent or in any of the polymers or oligomers recited for any of the general Formulae described herein (for example, in Formula I, Formula II, Formula III, or Formula IV), its definition on each occurrence is independent of its definition at every other occurrence. Also, combinations of substituents and/or variables are permissible only if such combinations result in stable compounds.

In some embodiments, the compounds are provided in the form of an acceptable salt (for example, a pharmaceutically acceptable salt) for treating microbial infections. A suitable salt that is considered to be acceptable is the hydrochloride acid addition salt. Since one or more of the disclosed compounds may be polyionic, such as a polyamine, the acceptable polymer or oligomer salt can be provided in the form of a poly (amine hydrochloride). Examples of other acceptable salts include, but are not limited to, those having sodium, potassium, or ammonium cations, and/or those having chloride, citrate, ascorbate, borate, phosphate, bicarbonate, sulfate, thiosulfate, bisulfite, mesylate, esylate, napsydisylate, tosylate, besylate, orthophoshate, acetate, gluconate, glutamate, lactate, malonate, fumarate, tartrate, maleate, or trifluoroacetate anions. In some embodiments, acceptable salts are those having mesylate, chloride, sulfate, esylate, napsydisylate, tosylate, besylate, phosphate, orthophoshate, acetate, gluconate, glutamate, lactate, malonate, citrate, fumarate, tartrate, maleate, or trifluoroacetate anions. In other embodiments, acceptable salts include sodium chloride, potassium chloride, sodium thiosulfate, sodium bisulfite, and ammonium sulfate.

In some embodiments, the compounds described herein are derivatives referred to as prodrugs. The expression “prodrug” denotes a derivative of a known direct acting drug. which derivative has enhanced delivery characteristics and therapeutic value as compared to the drug, and is transformed into the active drug by an enzymatic or chemical process.

The structures depicted herein may omit necessary hydrogen atoms to complete the appropriate valency. Thus, in some instances a carbon atom or nitrogen atom may appear to have an open valency (i.e., a carbon atom with only two bonds showing would implicitly also be bonded to two hydrogen atoms; in addition, a nitrogen atom with a single bond depicted would implicitly also be bonded to two hydrogen atoms). For example, “—N” would be considered by one skilled in the art to be “—NH₂.” Thus, in any structure depicted herein wherein a valency is open, one or more hydrogen atoms, as appropriate, is implicit, and is only omitted for brevity.

The present disclosure provides methods of treating or preventing a Cryptococcus fungal infection in a mammal comprising administering to the mammal in need thereof a compound of Formula I, Formula II, Formula III, or Formula IV. In some embodiments, the methods of treating or preventing a Cryptococcus fungal infection in a mammal comprise administering to the mammal in need thereof a compound of Formula I. In some embodiments, the methods of treating or preventing a Cryptococcus fungal infection in a mammal comprise administering to the mammal in need thereof a compound of Formula II. In some embodiments, the methods of treating or preventing a Cryptococcus fungal infection in a mammal comprise administering to the mammal in need thereof a compound of Formula III. In some embodiments, the methods of treating or preventing a Cryptococcus fungal infection in a mammal comprise administering to the mammal in need thereof a compound of Formula IV.

The present disclosure also provides methods of killing or inhibiting the growth of a Cryptococcus species comprising contacting the Cryptococcus species with a compound of Formula I, Formula II, Formula III, or Formula IV. In some embodiments, the methods of killing or inhibiting the growth of a Cryptococcus species comprise contacting the Cryptococcus species with a compound of Formula I. In some embodiments, the methods of killing or inhibiting the growth of a Cryptococcus species comprise contacting the Cryptococcus species with a compound of Formula II. In some embodiments, the methods of killing or inhibiting the growth of a Cryptococcus species comprise contacting the Cryptococcus species with a compound of Formula III. In some embodiments, the methods of killing or inhibiting the growth of a Cryptococcus species comprise contacting the Cryptococcus species with a compound of Formula IV.

The present disclosure provides methods of treating or preventing a fungal infection in a mammal comprising administering to the mammal in need thereof a compound of Formula I, Formula II, Formula III, or Formula IV in combination with one or more other anti-fungal agent(s) (i.e., in the same pharmaceutical composition or in separate pharmaceutical compositions). In some embodiments, the methods of treating or preventing a fungal infection in a mammal comprise administering to the mammal in need thereof a compound of Formula I in combination with one or more other anti-fungal agent(s) (i.e., in the same pharmaceutical composition or in separate pharmaceutical compositions). In some embodiments, the methods of treating or preventing a fungal infection in a mammal comprise administering to the mammal in need thereof a compound of Formula II in combination with one or more other anti-fungal agent(s) (i.e., in the same pharmaceutical composition or in separate pharmaceutical compositions). In some embodiments, the methods of treating or preventing a fungal infection in a mammal comprise administering to the mammal in need thereof a compound of Formula III in combination with one or more other anti-fungal agent(s) (i.e., in the same pharmaceutical composition or in separate pharmaceutical compositions). In some embodiments, the methods of treating or preventing a fungal infection in a mammal comprise administering to the mammal in need thereof a compound of Formula IV in combination with one or more other anti-fungal agent(s) (i.e., in the same pharmaceutical composition or in separate pharmaceutical compositions).

The present disclosure also provides methods of killing or inhibiting the growth of a fungus comprising contacting the fungus with a compound of Formula I, Formula II, Formula III, or Formula IV in combination with one or more other anti-fungal agent(s) (i.e., in the same pharmaceutical composition or in separate pharmaceutical compositions). In some embodiments, the methods of killing or inhibiting the growth of a fungus comprise contacting the fungus with a compound of Formula I in combination with one or more other anti-fungal agent(s) (i.e., in the same pharmaceutical composition or in separate pharmaceutical compositions). In some embodiments, the methods of killing or inhibiting the growth of a fungus comprise contacting the fungus with a compound of Formula II in combination with one or more other anti-fungal agent(s) (i.e., in the same pharmaceutical composition or in separate pharmaceutical compositions). In some embodiments, the methods of killing or inhibiting the growth of a fungus comprise contacting the fungus with a compound of

Formula III in combination with one or more other anti-fungal agent(s) (i.e., in the same pharmaceutical composition or in separate pharmaceutical compositions). In some embodiments, the methods of killing or inhibiting the growth of a fungus comprise contacting the fungus with a compound of Formula IV in combination with one or more other anti-fungal agent(s) (i.e., in the same pharmaceutical composition or in separate pharmaceutical compositions).

In some embodiments, the fungus is, or the fungal infection is caused by, Aspergillus spp. (e.g., Aspergillus fumigatus, Aspergillus favus, Aspergillus niger, and Aspergillus terreus), Fusarium spp. (e.g., Fusarium solani, Fusarium moniliforme, and Fusarium proliferartum), Malessezia spp. (e.g., Malessezia pachydermatis), Candida spp. (e.g., Candida albicans, Candida glabrata, Candida tropicalis, Candida krusei, and Candida auris), or Cryptococcus spp. (e.g., Cryptococcus neoformans), Mucorales such as Mucor spp. (e.g., M. circinelloides), Rhizopus spp. (e.g., Rhizopus delemar and Rhizopus oryzae), Lichtheimia spp. (e.g., Lichtheimia corymbifera), and Rhizomucor spp., or Chrysosporium parvum, Metarhizium anisopliae, Phaeoisaria clematidis, or Sarcopodium oculorum. In some embodiments, the fungus is, or the fungal infection is caused by, Aspergillus spp., Fusarium spp., Malessezia spp., Candida spp., or Cryptococcus spp. In some embodiments, the fungus is, or the fungal infection is caused by, Aspergillus spp. In some embodiments, the fungus is, or the fungal infection is caused by, Aspergillus fumigatus. In some embodiments, the fungus is, or the fungal infection is caused by, Aspergillus favus. In some embodiments, the fungus is, or the fungal infection is caused by, Aspergillus niger. In some embodiments, the fungus is, or the fungal infection is caused by, Aspergillus terreus. In some embodiments, the fungus is, or the fungal infection is caused by, Fusarium spp. In some embodiments, the fungus is, or the fungal infection is caused by, Fusarium solani. In some embodiments, the fungus is, or the fungal infection is caused by, Fusarium moniliforme. In some embodiments, the fungus is, or the fungal infection is caused by, Fusarium proliferartum. In some embodiments, the fungus is, or the fungal infection is caused by, Malessezia spp. In some embodiments, the fungus is, or the fungal infection is caused by, Malessezia pachydermatis. In some embodiments, the fungus is, or the fungal infection is caused by, a Mucorales. In some embodiments, the fungus is, or the fungal infection is caused by, Mucor spp. In some embodiments, the fungus is, or the fungal infection is caused by, M. circinelloides. In some embodiments, the fungus is, or the fungal infection is caused by, Rhizopus spp. In some embodiments, the fungus is, or the fungal infection is caused by, Rhizopus delemar. In some embodiments, the fungus is, or the fungal infection is caused by, Rhizopus oryzae. In some embodiments, the fungus is, or the fungal infection is caused by, Lichtheimia spp. In some embodiments, the fungus is, or the fungal infection is caused by, Lichtheimia corymbifera. In some embodiments, the fungus is, or the fungal infection is caused by, Rhizomucor spp. In some embodiments, the fungus is, or the fungal infection is caused by, Candida spp. In some embodiments, the fungus is, or the fungal infection is caused by, Candida albicans. In some embodiments, the fungus is, or the fungal infection is caused by, Candida glabrata. In some embodiments, the fungus is, or the fungal infection is caused by, Candida tropicalis. In some embodiments, the fungus is, or the fungal infection is caused by, Candida krusei. In some embodiments, the fungus is, or the fungal infection is caused by, Candida auris. In some embodiments, the fungus is, or the fungal infection is caused by, Cryptococcus spp. In some embodiments, the fungus is, or the fungal infection is caused by, Cryptococcus neoformans. In some embodiments, the fungus is, or the fungal infection is caused by, Chrysosporium parvum. In some embodiments, the fungus is, or the fungal infection is caused by, Metarhizium anisopliae. In some embodiments, the fungus is, or the fungal infection is caused by, Phaeoisaria clematidis. In some embodiments, the fungus is, or the fungal infection is caused by Sarcopodium oculorum.

Additional pathogenic fungi include the genus Candida (examples include C. albicans, C. glabrata, C. krusei, C. tropicalis, C. guilliermondii, C. parapsilosis, C. dubliniensis and C. auris), the genus Cryptococcus (examples include C. neoformans and C. gatti), the genus Trichosporon (examples include T. asahii, T. asteroides, T. cutaneum, T. dermatis, T. dohaense, T. inkin, T. loubieri, T. mucoides, and T. ovoides), the genus Malassezia (examples include M. globose and M. restricta), the genus Aspergillus (examples include A. fumigatus, A. flavis, A. terreu and A. niger), the genus Fusarium (examples include F. solani, F. falciforme, F. oxysporum, F. verticillioides, and F. proliferatum), the genus Mucor (examples include M. circinelloides, M. ramosissimus, M. indicus, M. rasemosus, and M. piriformis), the genus Blastomyces (examples include B. dermatitidis and B. brasiliensis), the genus Coccidioides (examples include C. immitis, and C. posadasii), the genus Pneumocystis (examples include P. carinii and P. jiroveci), the genus Histoplasma (examples include H. capsulatum), the genus Trichophyton (examples include T. schoenleinii, T. mentagrophytes, T. verrucosum, and T. rubrum), the genus Rhizopus (examples include R. oryzae and R. stolonifera), the genus Apophysomyces (examples include A. variabilis), the genus Rhizomucor (examples include R. pusillus, R. regularior, and R. chlamydosporus), the genus Lichtheimia (examples include L. ramose and L. corymbifera), the genus Scedosporium (examples include S. apiospermum), and the genus Lomentospora (examples include L. prolificans).

In some embodiments, the fungi is Mucorales (for which conventional therapy results are poor), and other lethal pathogens for which current therapy is poor or lacking (Fusarium, Scedosporium, Lomentospora, Acremonium, and Exserohilum).

In some embodiments, the fungal species is resistant to a therapeutic agent. In some embodiments, the fungal species is resistant to an azole. In some embodiments, the fungal species is resistant to an echinocandin. In some embodiments, the fungal species is CAS-resistant. In some embodiments, the fungal species is VOR-resistant.

In any of the methods described herein, the compound of Formula I comprises:

R¹—[—X-A₁-X—Y-A₂-Y—]ₘ—R² (I),
or a pharmaceutically acceptable salt thereof, wherein:

X is NR⁸, O, S, —N(R⁸)N(R⁸)—, —N(R⁸)—(N═N)—, —(N═N)—N(R⁸)—, —C(R⁷R^{7′})NR⁸—, —C(R⁷R^{7′})O—, or —C(R⁷R^{7′})S—; and

Y is C═O, C═S, O⊚S═O, —C(═O)C(═O)—, C(R⁶R^{6′})C═O or C(R⁶R^{6′})C═S; or

X and Y are taken together are pyromellitic diimide;

R⁸ is hydrogen or alkyl;

R⁷ and R^{7′} are, independently, hydrogen or alkyl, or R⁷ and R^{7′} together are —(CH₂)ₚ-, wherein p is 4 to 8; and

R⁶ and R^{6′} are, independently, hydrogen or alkyl, or R⁶ and R^{6′} together are (CH₂)₂NR¹²(CH₂)₂, wherein R¹² is hydrogen, —C(═N)CH₃ or C(═NH)—NH₂;

A₁ and A₂ are, independently, optionally substituted arylene or optionally substituted heteroarylene, wherein A₁ and A₂ are, independently, optionally substituted with one or more polar (PL) group(s), one or more non-polar (NPL) group(s), or a combination of one or more polar (PL) group(s) and one or more non-polar (NPL) group(s);

R¹ is:
(i) hydrogen, a polar group (PL), or a non-polar group (NPL), and R² is —X-A₁-X—R¹, wherein A₁ is as defined above and is optionally substituted with one or more polar (PL) group(s), one or more non-polar (NPL) group(s), or a combination of one or more polar (PL) group(s) and one or more non-polar (NPL) group(s);
(ii) hydrogen, a polar group (PL), or a non-polar group (NPL), and R² is —X-A′-X—R¹, wherein A′ is aryl or heteroaryl and is optionally substituted with one or more polar (PL) group(s), one or more non-polar (NPL) group(s), or a combination of one or more polar (PL) group(s) and one or more non-polar (NPL) group(s);

(iii) —Y-A₂-Y—R², and R² is hydrogen, a polar group (PL), or a non-polar group (NPL);

(iv) —Y-A′ and R² is —X-A′, wherein A′ is aryl or heteroaryl and is optionally substituted with one or more polar (PL) group(s), one or more non-polar (NPL) group(s), or a combination of one or more polar (PL) group(s) and one or more non-polar (NPL) group(s);

(v) R¹ and R² are, independently, a polar group (PL) or a non-polar group (NPL); or
(vi) R¹ and R² together form a single bond;

NPL is a nonpolar group independently selected from —B(OR⁴), and —(NR³′)_{q1NPL}-U^{NPL}-(CH₂)_{pNPL}-(NR^{3″})_{q2NPL}-R^{4′}, wherein:
R³, R³′, and R³″ are independently selected from hydrogen, alkyl, and alkoxy;
R⁴ and R^{4′} are, independently, selected from hydrogen, alkyl, alkenyl, alkynyl, cycloalkyl, aryl, and heteroaryl, any of which is optionally substituted with one or more alkyl or halo groups;
U^{NPL} is absent or selected from O, S, S(═O), S(═O)2, NR³, —C(═O)—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O),O—, —R³⁰—, —R³S—, —S—C═N—, and —C(═O—NR³—O—, wherein groups with two chemically nonequivalent termini can adopt both possible orientations;
the —CH₂)_{pNPL}- alkylene chain is optionally substituted with one or more alkyl, amino, or hydroxy groups, or is unsaturated;
pNPL is 0 to 8;
q1NPL and q2NPL are, independently, 0, 1, or 2;

PL is a polar group selected from halo, hydroxyethoxymethyl, methoxyethoxymethyl, polyoxyethylene, and —(NR^{5′})_{q1PL}-U^{PL}-(CH₂)_{pPL}-(NR^{5″})_{q2PL}-V, wherein:
R⁵, R^{5′}, and R^{5″} are, independently, selected from hydrogen, alkyl, and alkoxy;
U^{PL} is absent or selected from O, S, S(═O), S(═O)₂, NR⁵, —C(═O)—, —C(═O)—N═N—NR⁵—, —C(═O)—NR⁵—N═N—, —N=N—NR⁵—, —C(=N—N(R⁵)₂)—, —C(=NR⁵)—, —C(═O)O—, —C(═O)S—, —C(=S)—, —O—P(═O)₂O—, —R⁵O—, —R⁵S—, —S—C═N— and —(═O—NR⁵—O—, wherein groups with two chemically nonequivalent termini can adopt both possible orientations;
V is selected from the group consisting of nitro, cyano, amino, hydroxy, alkoxy, alkylthio, alkylamino, dialkylamino, —NH(CH₂)ₚNH₂ wherein p is 1 to 4, —N(CH₂CH₂NH₂)₂, diazamino, amidino, guanidino, guanyl, semicarbazone, aryl, heterocycle, and heteroaryl, any of which is optionally substituted with one or more of amino, halo, cyano, nitro, hydroxy, —NH(CH₂)ₚNH₂ wherein p is 1 to 4, —N(CH₂CH₂NH₂)₂, amidino, guanidino, guanyl, aminosulfonyl, aminoalkoxy, aminoalkythio, lower acylamino, or benzyloxycarbonyl;
the —(CH₂)_{pPL}- alkylene chain is optionally substituted with one or more amino or hydroxy groups, or is unsaturated;
pPL is 0 to 8;
q1PL and q2PL are, independently, 0, 1, or 2; and
m is 1 to about 500.

In any of the methods described herein, the compound of Formula II comprises:

R¹—X-A₁-X—Y-A₂-Y—X-A₁-X—R² (II),
or a pharmaceutically acceptable salt thereof, wherein:

X is NR⁸, O, S, or —N(R⁸)N(R⁸)—; and Y is C—O, C═S, or O═S═O, wherein R⁸ is hydrogen or alkyl;

A₁ and A₂ are, independently, optionally substituted arylene or optionally substituted heteroarylene, wherein A₁ and A₂ are, independently, optionally substituted with one or more polar (PL) group(s), one or more non-polar (NPL) group(s), or a combination of one or more polar (PL) group(s) and one or more non-polar (NPL) group(s);

R¹ is a polar group (PL) or a non-polar group (NPL); and R² is R¹;

NPL is a nonpolar group —NR^{3′})_{q1NPL}-U^{NPL}-(CH₂)_{pNPL}-(NR^{3′})_{q2NPL}-R^{4′}, wherein:
R³, R³′, and R³″ are, independently, selected from hydrogen, alkyl, and alkoxy;
R⁴ and R^{4′} are, independently, selected from hydrogen, alkyl, alkenyl, alkynyl, cycloalkyl, aryl, and heteroaryl, any of which is optionally substituted with one or more alkyl or halo groups;
U^{NPL} is absent or selected from O, S, S(═O), S(═O)₂, NR³, —C(═O)—, C(═O)—N—N—NR³—, —C(═O)—NR³—N═N—, —N═N—NR³—, —C(═N—N(R³)₂)—, —C(═NR³)—, —C(═O)O—, —C(═O)S—, —C(′S)—, —O—P(═O),O—, —R³O—, —R³S—, —S—C═N—, and —C(—O)—NR³—O—, wherein groups with two chemically nonequivalent termini can adopt both possible orientations;
the —(CH₂)_{pNPL}- alkylene chain is optionally substituted with one or more alkyl, amino, or hydroxy groups, or is unsaturated;
pNPL is 0 to 8;
q1NPL and q2NPL are, independently, 0, 1, or 2;

PL is a polar group selected from halo, hydroxyethoxymethyl, methoxyethoxymethyl, polyoxyethylene, and —NR^{5′})_{q1PL}-U^{PL}-(CH₂)_{pPL}-(NR^{5′})_{q2PL}-V, wherein:
R⁵, R⁵′, and R^{5″} are, independently, selected from hydrogen, alkyl, and alkoxy;
U^{PL} is absent or selected from O, S, S(═O), S(═O)₂, NR⁵, —C(═O)—, —C(═O)—N═N—NR⁵—, —C(═O)—NR⁵—N—N—, —N═N—NR⁵—, —C(═N—N(R⁵)₂)—, —C(═NR⁵)—, —C(═O)O—, —C(═O)S—, —C(═S)—, —O—P(═O),O—, —R⁵O—, —R⁵S—, —S—C═N—, and —C(═O)—NR⁵—O—, wherein groups with two chemically nonequivalent termini can adopt both possible orientations;
V is selected from nitro, cyano, amino, hydroxy, alkoxy, alkylthio, alkylamino, dialkylamino, —NH(CH₂)ₚNH₂ wherein p is 1 to 4, —N(CH₂CH₂NH₂)₂, diazamino, amidino, guanidino, guanyl, semicarbazone, aryl, heterocycle, and heteroaryl, any of which is optionally substituted with one or more of amino, halo, cyano, nitro, hydroxy,—NH(CH₂)ₚNH₂ wherein p is 1 to 4, —N(CH₂CH₂NH₂)₂, amidino, guanidino, guanyl, aminosulfonyl, aminoalkoxy, aminoalkythio, lower acylamino, or benzyloxycarbonyl;
the —(CH₂)_{pPL}- alkylene chain is optionally substituted with one or more amino or hydroxy groups, or is unsaturated;
pPL is 0 to 8; and
q1PL and q2PL are, independently, 0, 1, or 2.

In some embodiments, in the compound of Formula II, X is NR⁸ and Y is C═O. For example, X is NH and Y is C═O.

In some embodiments, in the compound of Formula II, A₁ and A₂ are independently optionally substituted o-, m-, or p-phenylene. In some embodiments, A₁ and A₂ are optionally substituted m-phenylene.

In some embodiments, in the compound of Formula II, one of A₁ and A₂ is o-, m-, or p-phenylene, and the other of A₁ and A₂ is o-, m-, or p-heteroarylene. In some embodiments, in the compound of Formula II, the heteroarylene groups include, but are not limited to, pyridinylene, pyrimidinylene, and pyrazinylene. In some embodiments, in the compound of Formula II, the heteroarylene group is pyrimidinylene, in particular, m-pyrimidinylene.

In some embodiments, in the compound of Formula II, A₁ and A₂ are, independently, optionally substituted arylene or optionally substituted heteroarylene, and (i) one of A₁ and A₂ is substituted with one or more polar (PL) group(s) and one or more nonpolar (NPL) group(s) and the other of A₁ and A₂ is unsubstituted; or (ii) one of A₁ and A₂ is substituted with one or more polar (PL) group(s) and one or more nonpolar (NPL) group(s) and the other of A₁ and A₂ is substituted with one or more polar (PL) group(s). Especially preferred are oligomers in which either (i) one of A₁ and A₂ is substituted with one polar (PL) group and one nonpolar (NPL) group, and the other of A₁ and A₂ is unsubstituted, or (ii) one of A₁ and A₂ is substituted with one polar (PL) group and one nonpolar (NPL) group and the other of A₁ and A₂ is substituted with one or two polar (PL) group(s), as defined above.

In some embodiments, in the compound of Formula II, R¹ is hydrogen or a polar group (PL). In some embodiments, in the compound of Formula II, R¹ is —(NR^{5′})_{q1PL}-U^{PL}-(CH₂)_{pPL}-(NR^{5′})_{q2PL}-V, wherein R⁵, R⁵, R^{5″}, V, U^{PL}, and pPL are as defined above, and q1PL and q2PL are each 0. In some embodiments, in the compound of Formula II, R¹is -U^{PL}-(CH₂)_{pPL}-V. In some embodiments, in the compound of Formula II, U^{PL} is absent or is O, S, NH, —C(═O)O—, or —C(═O); pPL is 0 to 6, especially 1 to 4; and V is amino, aminoalkyl, amidino, guanidino, aryl, or heteroaryl optionally substituted with one or more amino, guanidino, amidino, or halo groups.

In some embodiments, in the compound of Formula II, each of R³, R^{3′}, and R^{3″} are hydrogen, C₁-C₆ alkyl, and C₁-C₆ alkoxy. In some embodiments, in the compound of Formula II, R³, R^{3′}, and R^{3″} are each hydrogen.

In some embodiments, in the compound of Formula II, R^{4′} is hydrogen or alkyl optionally substituted with one or more alkyl or halo groups. In some embodiments, in the compound of Formula II, R^{4′} is hydrogen, C₁-C₁₀ alkyl, C₃-C₁₈ branched alkyl, C₂-C₁₀ alkenyl, C₂-C₁₀ alkynyl, or C₆-C₁₀ aryl. In some embodiments, in the compound of Formula II, R^{4′} is phenyl. In some embodiments, in the compound of Formula II, R^{4′} is C₁-C₁₀ alkyl or C₃-C₁₈ branched alkyl. In some embodiments, in the compound of Formula II, C₁-C₁₀ alkyl and C₃-C₁₈ branched alkyl groups are methyl, ethyl, n-propyl, isopropyl, n-butyl, tert-butyl, and n-pentyl.

In some embodiments, in the compound of Formula II, U^{NPL} is absent. In some embodiments, in the compound of Formula II, U^{NPL} is O, S, NH, —C(═O)—, —C(═O)O—, —R³S—, or —R³O—. In some embodiments, in the compound of Formula II, U^{NPL} is O, —C(═O)—, or —C(═O)O—.

In some embodiments, in the compound of Formula II, pNPL is 0 to 6. In some embodiments, in the compound of Formula II, pNPL is 0 to 4, In some embodiments, in the compound of Formula II, pNPL is 0, 1, or 2.

In some embodiments, in the compound of Formula II, q1NPL and q2NPL are each, independently, 0 or 1. In some embodiments, in the compound of Formula II, q1NPL and q2NPL are, independently, 0 or 1. In some embodiments, in the compound of Formula II, q1NPL and q2NPL are both 0.

In some embodiments, in the compound of Formula II, the —(CH₂)_{pNPL}- alkylene chain in NPL is unsubstituted or substituted with one or more alkyl groups.

In some embodiments, in the compound of Formula II, NPL is C₁-C₆ alkyl optionally substituted with one or more halo groups. In some embodiments, in the compound of Formula II, NPL is n-propyl, isopropyl, n-butyl, tert-butyl, or trifluoromethyl.

In some embodiments, in the compound of Formula II, PL is —(NR^{5′})_{q1PL}-U^{PL}-(CH₂)_{pPL}-(NR^{5′})_{q2PL}-V, and R⁵, R^{5′}, R^{5″}, V, U^{PL}, pPL, q1PL and q2PL are as defined above.

In some embodiments, in the compound of Formula II, R⁵, R^{5′}, and R^{5″} are each, independently, hydrogen, C₁-C₆ alkyl, or C₁-C₆ alkoxy. In some embodiments, in the compound of Formula II, each of R⁵, R^{5′}, and R^{5″} is hydrogen.

In some embodiments, in the compound of Formula II, U^{PL} is O, S, NR⁵, —C(═O)—, —C(═O)—N═N—NH—, —C(═O)—NH—N═N—, —N═N—NH—, —C(═N—N(R⁵)₂)—, —C(═NR⁵)—, —C(′O)O—, —R⁵S—, or —R⁵O—, wherein R⁵ is hydrogen. In some embodiments, in the compound of Formula II, U^{PL} is O, S, NH, —C(═O)O—, or —C(═O). In some embodiments, in the compound of Formula II, U^{PL} is absent.

In some embodiments, in the compound of Formula II, V is nitro, cyano, amino, hydroxy, C₁-C₆ alkoxy, C₁-C₆ dialkylamino, C₁-C₆ alkylthio, C1-C6 alkylamino, diazamino, amidino, guanidino, guanyl, semicarbazone, C₆-C₁₀ aryl, heterocycle, —NH(CH₂)ₚNH₂ wherein p is 1 to 4, heteroaryl, or —N(CH₂CH₂NH₂)₂, any of which is optionally substituted with one or more of amino, halo, cyano, nitro, hydroxy, —NH(CH₂)ₚNH₂ wherein p is 1 to 4, —N(CH₂CH₂NH₂)₂, amidino, guanidino, guanyl, aminosulfonyl, aminoalkoxy, lower acylamino, or benzyloxycarbonyl. Suitable heteroaryl groups include, but are not limited to, indolyl, 3H-indolyl, 1H-isoindolyl, indazolyl, benzoxazolyl, pyridyl, and 2-aminopyridyl. Suitable heterocycle groups also include, but are not limited to, piperidinyl, piperazinyl, imidazolidinyl, pyrrolidinyl, pyrazolidinyl, and morpholinyl. In some embodiments, in the compound of Formula II, V is amino, C₁-C₆ alkylamino, —NH(CH₂)ₚNH₂ wherein p is 1 to 4, —N(CH₂CH₂NH₂)₂, diazamino, amidino, or guanidino, any of which can be optionally substituted with one or more of amino, halo, cyano, nitro, hydroxy, —NH(CH₂)ₚNH₂ wherein p is 1 to 4, —N(CH₂CH₂NH₂)₂, amidino, guanyl, guanidino, or aminoalkoxy. In some embodiments, in the compound of Formula II, V is amino or guanidino.

In some embodiments, in the compound of Formula II, pPL is 0 to 6. In some embodiments, in the compound of Formula II, pPL is 0 to 4. In some embodiments, in the compound of Formula II, pPL is 2 to 4.

In some embodiments, in the compound of Formula II, q1PL and q2PL are, independently, 0 or 1. In some embodiments, in the compound of Formula II, q1PL and q2PL are, independently, 0 or 1. In some embodiments, in the compound of Formula II, each of q1PL and q2PL is 0.

In some embodiments, in the compound of Formula II, the —(CH₂)_{pPL}- alkylene chain in PL is optionally substituted with one or more amino or hydroxy groups.

In some embodiments, in the compound of Formula II: X is NR⁸, and Y is C═O; wherein R⁸ is hydrogen or (C₁-C₄)alkyl; A₁ and A₂ are, independently, optionally substituted phenylene or optionally substituted pyrimidinylene, wherein A₁ is substituted with one or more polar (PL) group(s) and one or more non-polar (NPL) group(s), and A₂ is substituted with one or more polar (PL) group(s) or is unsubstituted; R¹ is a polar group (PL); and R² is R¹; NPL is a nonpolar group —(NR^{3′})_{q1NPL}-U^{NPL}-(CH₂)_{pNPL}-(NR^{3′})_{q2NPL}-R^{4′}, wherein: R⁴ and R^{4′} are, independently, selected from hydrogen and alkyl optionally substituted with one or more alkyl or halo groups; U^{NPL} is absent or selected from O, S, NR³, and —C(═O)—; pNPL is 0 to 6; q1NPL and q2NPL are, independently, 0; PL is a polar group —(NR^{5′})_{q1PL}-U^{PL}-(CH₂)_{pPL}-(NR^{5′})_{q2PL}-V, wherein: U^{PL} is absent or selected from O, S, NR⁵, and —C(═O)—; V is selected from amino, alkylamino, dialkylamino, —NH(CH₂)ₚNH₂ wherein p is 1 to 4, —N(CH₂CH₂NH₂)₂, diazamino, amidino, and guanidino, any of which is optionally substituted with one or more of amino, halo, —NH(CH₂)ₚNH₂ wherein p is 1 to 4, —N(CH₂CH₂NH₂)₂, amidino, guanidino, guanyl, aminosulfonyl, aminoalkoxy, aminoalkythio, and lower acylamino; pPL is 0 to 8; and q1PL and q2PL are, independently, 0.

In some embodiments, in the compound of Formula II: A₁ is phenylene substituted with one (PL) group and one non-polar (NPL) group, and A₂ is unsubstituted pyrimidinylene or pyrimidinylene substituted with one or two polar (PL) group(s); NPL is R^{4′}, wherein R^{4′} is (C₁-C₆)alkyl optionally substituted with one or more halo groups; PL is -U^{PL}-(CH₂)_{pPL}-V, wherein: U^{PL} is O or S; V is selected from amino, amidino, and guanidino; and pPL is 0 to 6.

In some embodiments, in the compound of Formula II: A₁ is phenylene substituted with one (PL) group and one non-polar (NPL) group, and A₂ is unsubstituted phenylene or phenylene substituted with one or two polar (PL) group(s); NPL is R^{4′}, wherein R^{4′} is (C₁-C₆)alkyl optionally substituted with one or more halo groups; PL is -U^{PL}-(CH₂)_{pPL}-V, wherein: U^{PL} is O or S; V is selected from amino, amidino, and guanidino; and pPL is 0 to 6.

In some embodiments, in the compound of Formula II, A₁ is phenylene substituted with one (PL) group and one non-polar (NPL) group, and A₂ is phenylene substituted with one or two polar (PL) group(s).

In some embodiments, in the compound of Formula II, A₁ is phenylene substituted with one (PL) group and one non-polar (NPL) group, and A₂ is unsubstituted phenylene.

In any of the methods described herein, the compound of Formula III comprises:

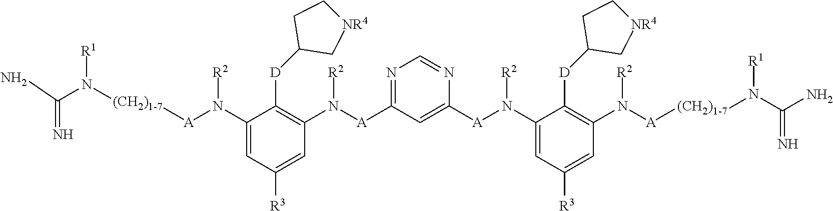

or a pharmaceutically acceptable salt thereof, wherein: each A is, independently, —C═O, —C═S, or CH₂; each D is, independently, O or S; each R¹ is, independently, hydrogen, C₁₋₃ alkyl, C₁₋₃ alkoxy, halo, or halo C₁₋₃ alkyl; each R² is, independently, hydrogen, C₁₋₃ alkyl, C₁₋₃ alkoxy, halo, or halo C₁₋₃ alkyl; each R³ is, independently, hydrogen, C₁₋₄alkyl, C₁₋₄alkoxy, halo, or halo C₁₋₄alkyl; and each R⁴ is, independently, hydrogen, C₁₋₃ alkyl, C₁₋₃ alkoxy, halo, or halo C₁₋₃ alkyl.

In some embodiments, at least one A is —C—O. In some embodiments, each A is —C═O.

In some embodiments, at least one D is O. In some embodiments, each D is O.

In some embodiments, each R¹ is, independently, hydrogen, methyl, ethyl, methoxy, ethoxy, halo, or haloC₁₋₃ alkyl. In some embodiments, each R¹ is, independently, hydrogen, methyl, methoxy, halo, or haloC₁₋₃ alkyl. In some embodiments, each R¹ is, independently, hydrogen, methyl, or methoxy. In some embodiments, at least one R¹ is hydrogen. In some embodiments, each R¹ is hydrogen.

In some embodiments, each R² is, independently, hydrogen, methyl, ethyl, methoxy, ethoxy, halo, or haloC₁₋₃ alkyl. In some embodiments, each R² is, independently, hydrogen, methyl, methoxy, or halo. In some embodiments, at least one R² is hydrogen. In some embodiments, each R² is hydrogen.

In some embodiments, each R³ is, independently, hydrogen, methyl, ethyl, methoxy, ethoxy, halo, or haloC₁₋₃ alkyl. In some embodiments, each R³ is, independently, methyl, methoxy, halo, or haloC₁₋₃ alkyl. In some embodiments, each R³ is, independently, halo or haloC₁₋₃ alkyl. In some embodiments, each R³ is, independently, haloC₁₋₃ alkyl. In some embodiments, at least one R³ is trifluoromethyl. In some embodiments, each R³ is trifluoromethyl.

In some embodiments, each R⁴ is, independently, hydrogen, methyl, ethyl, methoxy, ethoxy, or haloC₁₋₃ alkyl. In some embodiments, each R⁴ is, independently, hydrogen, methyl, methoxy, halo, or haloC₁₋₃ alkyl. In some embodiments, each R⁴ is, independently, hydrogen, methyl, methoxy, or halo. In some embodiments, at least one R⁴ is hydrogen. In some embodiments, each R⁴ is hydrogen.

In some embodiments, each A is, independently, —C═O or —C═S; each D is, independently, O or S; each R¹ is, independently, hydrogen, methyl, ethyl, methoxy, ethoxy, halo, halomethyl, or haloethyl; each R² is, independently, hydrogen, methyl, methoxy, halo, or halomethyl; each R³ is, independently, C₁₋₃ alkyl, C₁₋₃ alkoxy, halo, or haloalkyl; and each R⁴ is, independently, hydrogen, methyl, ethyl, methoxy, ethoxy, halo, halomethyl, or haloethyl.

In some embodiments, each A is, independently, —C═O or —C═S; each D is, independently, O or S; each R¹ is, independently, hydrogen, methyl, methoxy, halo, or halomethyl; each R² is, independently, hydrogen, halo, or halomethyl; each R³ is, independently, methyl, ethyl, methoxy, ethoxy, halo, halomethyl, or haloethyl; and each R⁴ is, independently, hydrogen, methyl, ethyl, methoxy, ethoxy, halo, halomethyl, or haloethyl.

In some embodiments, each A is —C—O; each D is O; each R¹ is, independently, hydrogen, halo, or halomethyl; each R² is, independently, hydrogen or halo; each R³ is, independently, methyl, methoxy, halo, or halomethyl; and each R⁴ is, independently, hydrogen, methyl, methoxy, halo, or halomethyl.

In some embodiments, each A is —C—O; each D is O; each R¹ is, independently, hydrogen or halo; each R² is, independently, hydrogen or halo; each R³ is, independently, methyl, halo, or halomethyl; and each R⁴ is, independently, hydrogen, methyl, halo, or halomethyl.

In some embodiments, each A is —C—O; each D is O; each R¹ is, independently, hydrogen or halo; each R² is, independently, hydrogen or halo; each R³ is, independently, halo or halomethyl; and each R⁴ is, independently, hydrogen or halo.

In some embodiments, each A is —C═O; each D is O; each R¹ is, independently, hydrogen or halo; each R² is, independently, hydrogen or halo; each R³ is, independently, methyl, halo, or halomethyl; and each R⁴ is, independently, hydrogen, methyl, halo, or halomethyl.

In some embodiments, each A is —C—O; each D is O; each R¹ is, independently, hydrogen or halo; each R² is, independently, hydrogen or halo; each R³ is, independently, halo or halomethyl; and each R⁴ is, independently, hydrogen, halo, or halomethyl.

In any of the methods described herein, the compound of Formula IV comprises:

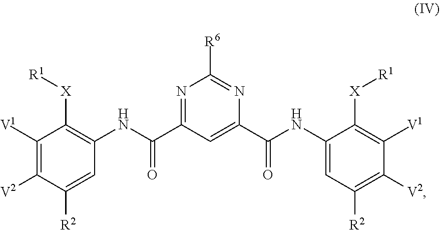

or a pharmaceutically acceptable salt thereof, wherein:

each X is, independently, O, S, or S(═O)2;

each R¹ is, independently, —CH₃, —(CH₂)ₙ—NH₂, —(CH₂)ₙ—NH—C(═NH)NH₂, or —(CH₂)ₙ—NH—C(═O)—R⁴, where each n is, independently, 1 to 4, and each R⁴ is, independently, H, -C₁-C₃alkyl, or —(CH₂)ₚ—NH₂, where each p is, independently, 1 or 2;

each R² is, independently, H, halo, —CF₃, or —C(CH₃)₃;

each V₂ is H, and each V₁ is, independently, —N—C(—O)—R³, where each R³ is, independently, —(CH₂)ₙ—NH₂ or —(CH₂)ₙ—NH—C(═NH)NH₂, where each n is, independently, 1 to 4; or each V₁ is H and each V² is, independently, —S—R⁵, where each R⁵ is, independently, —(CH₂)ₙ—NH₂ or —(CH₂)ₙ—NH—C(═NH)NH₂, where each n is, independently, 1 to 4; and

each R⁶ is H, —S—(CH₂)ₘ—NH₂, —S—(CH₂)ₘ—NH—C(═NH)NH₂, —O—(CH₂)ₘ—NH₂, or —O—(CH₂)ₘ—NH═C(=NH)NH₂, where each m is, independently, 1 to 4.

In some embodiments, each X is S.

In any of the above embodiments for a compound comprising Formula IV, each R¹ is, independently, —CH₃, —(CH₂)ₙ—NH₂, —(CH₂)ₙ—NH—C(═NH)NH₂, or —(CH₂)ₙ—NH—C(═O)—R⁴, where each n is, independently, 1 or 2, and each R⁴ is, independently, H or methyl; or each R¹ is, independently, —CH₃, —(CH₂)ₙ—NH₂, —(CH₂)ₙ—NH—C(═NH)NH₂, or —(CH₂)ₙ—NH—C(═O)—R⁴, where each n is 2 and each R⁴ is H; or each R¹ is, independently, —(CH₃, —(CH₂)ₙ—NH₂ or —(CH₂)ₙ—NH—C(═NH)NH₂, where each n is 2; or each R¹ is —CH₃, —(CH₂)ₙ—NH₂, or —(CH₂)ₙ—NH—C(═NH)NH₂, where each n is 2; or each R¹ is —CH₃ or —(CH₂)ₙ—NH₂ where each n is 2.

In any of the above embodiments for a compound comprising Formula IV, each R² is, independently, H, Br, F, Cl, —CF3, or —-C(CH₃)₃; or each R² is, independently, Br, F, Cl, —CF₃, or —C(CH₃)₃; or each R² is —CF₃.

In any of the above embodiments for a compound comprising Formula IV, each V² is H and each V¹ is, independently, —N—C(—O)—R³, where each R³ is, independently, —(CH₂)ₙ—NH₂ or —(CH₂)ₙ—NH—C(═NH)NH₂, where each n is, independently, 1 to 4; or each V² is H and each V¹ is, independently, —N—C(═O)—R³, where each R³ is, independently, —(CH₂)ₙ—NH₂ or —(CH₂)ₙ—NH—C(═NH)NH₂, where each n is, independently, 1 or 2; or each V² is H and each V¹ is, independently, —N—C(═O)—R³, where each R³ is, independently, —(CH₂)ₙ—NH₂ or —(CH₂)ₙ—NH—C(═NH)NH₂, where each n is 2; or each V² is H and each V¹ is —N—C(═O)—R³, where each R³ is —(CH₂)ₙ—NH₂ or —(CH₂)ₙ—NH—C(═NH)NH₂, where n is 2.

In any of the above embodiments for a compound comprising Formula IV, each V¹ is H and each V² is, independently, —S—R⁵, where each R⁵ is, independently, —(CH₂)ₙ—NH₂ or —(CH₂)ₙ—NH—C(═NH)NH₂, where each n is, independently, 1 to 4; or each V¹ is H and each V² is, independently, —S—R⁵, where each R⁵ is, independently, —(CH₂)ₙ—NH₂ or —(CH₂)ₙ—NH—C(═NH)NH₂, where each n is 1 or 2; or each V¹ is H and each V² is, independently, —S—R⁵, where each R⁵ is, independently, —(CH₂)ₙ—NH₂ or —(CH₂)ₙ—NH—C(═NH)NH₂, where each n is 2; or each V¹ is H and each V² is —S—R⁵, where each R⁵ is —(CH₂), —NH₂ or —(CH₂)ₙ—NH—C(═NH)NH₂, where each n is 2; or each V¹ is H and each V² is —S—R⁵, where each R⁵ is —(CH₂)ₙ—NH₂ where each n is 2.

In any of the above embodiments for a compound comprising Formula IV, each R⁶ is H, —S—(CH₂)ₘ—NH₂, or —S—(CH₂)ₘ—NH—C(═NH)NH₂, where each m is, independently, 1 to 4; or each R⁶ is H, —S—(CH₂)ₘ—NH₂, or —S—(CH₂)ₘ—NH—C(═NH)NH₂, where each m is, independently, 1 or 2; or each R⁶ is H or —S—(CH₂)ₘ—NH—C(═NH)NH₂, where each m is, independently, 1 or 2; or each R⁶ is H or —S—(CH₂)ₘ—NH—C(═NH)NH₂, where each m is 2.

In some embodiments, each X is S; each R¹ is, independently, —(CH₂)ₙ—NH₂ or —(CH₂)ₙ—NH—C(═NH)NH₂, where each n is, independently, 1 to 4; each R² is, independently, halo, —CF₃, or —C(CH₃)₃; and each V¹ is H and each V² is, independently, —S—R⁵, where each R⁵ is, independently, —(CH₂), —NH₂, where each n is, independently, 1 to 4.

In some embodiments, each X is S; each R¹ is, independently, —(CH₂)ₙ—NH₂, where each n is, independently, 1 or 2; each R² is, independently, —CF₃ or —C(CH₃)₃; and each V¹ is H and each V² is, independently, —S—R⁵, where each R⁵ is, independently, —(CH₂)ₙ—NH₂, where each n is, independently, 1 or 2.

In some embodiments, each X is S; each R¹ is —(CH₂)ₙ—NH₂, where each n is 1 or 2; each R² is, independently, —CF₃ or —C(CH₃)₃; and each V¹ is H and each V² is —S—R⁵, where each R⁵ is —(CH₂)ₙ—NH₂, where each n is 1 or 2. In some embodiments, each X is O or S; each R¹ is, independently, —(CH₂)ₙ—NH₂, or —(CH₂)ₙ—NH—C(═NH)NH₂, or —(CH₂)ₙ—NH—C(═O)—R⁴, where each n is, independently, 1 to 4, and each R⁴ is, independently, H or methyl; each R² is, independently, halo, —CF₃, or —C(CH₃)₃; and each V² is H, and each V¹ is, independently, —N—C(—O)—R³, where each R³ is, independently, —(CH₂)ₙ—NH₂ or —(CH₂)ₙ—NH—C(═NH)NH₂, where each n is, independently, 1 to 4.

In some embodiments, each X is S; each R¹ is, independently, —(CH₂)ₙ—NH—C(═O)—R⁴, where each n is, independently, 1 or 2, and each R⁴ is, independently, H or methyl; each R² is, independently, halo; and each V² is H, and each V¹ is —N—C(—O)—R³, where each R³ is —(CH₂)ₙ—NH₂ or —(CH₂)ₙ—NH—C(═NH)NH₂, where each n is 4.

In some embodiments, each X is O or S; each R¹ is, independently, —(CH₂)ₙ—NH₂ or —(CH₂)ₙ—NH—C(═NH)NH₂, where each n is, independently, 1 to 4; each R² is, independently, halo, —CF₃, or —C(CH₃)₃; and each V² is H, and each V¹ is, independently, —N—C(═O)—R³, where each R³ is, independently, —(CH₂)ₙ—NH₂ or —(CH₂)ₙ—NH—C(═NH)NH₂, where each n is, independently, 1 to 4.

In some embodiments, each X is O or S; each R¹ is —(CH₂)ₙ—NH₂ or —(CH₂)ₙ—NH—C(═NH)NH₂, where each n is 1 or 2; each R² is halo, —CF₃, or —C(CH₃)₃; and each V² is H, and each V¹ is —N—C(═O)—R³, where each R³ is —(CH₂)ₙ—NH₂ or —(CH₂)ₙ—NH—C(═NH)NH₂, where each n is 3 or 4.

In some embodiments, each X is, independently, S or S(═O)₂; each R¹ is, independently, —(CH₂)ₙ—NH₂ or —(CH₂)ₙ—NH—C(═O)—R⁴, where each n is, independently, 1 or 2, and each R⁴ is, independently, —(CH₂)ₚ—NH₂, where each p is, independently, 1 or 2; each R² is, independently, halo or —CF₃; and each V² is H, and each V¹ is, independently, —N—C(═O)—R³, where each R³ is, independently, —(CH₂)ₙ—NH₂ or —(CH₂)ₙ—NH—C(═NH)NH₂, where each n is, independently, 3 or 4.

In some embodiments, each X is O or S; each R¹ is —CH₃; each R² is —CF₃; each V¹ is H and each V² is, independently, —S—R⁵, where each R⁵ is, independently, —(CH₂)ₙ—NH₂ or —(CH₂)ₙ—NH—C(═NH)NH₂, where each n is, independently, 1 to 4; and each R⁶ is —S—(CH₂)ₘ—NH₂ or —S—(CH₂)ₘ—NH—C(═NH)NH₂, where each m is, independently, 1 or 2.

In some embodiments. the compound comprising Formula IV is chosen from:

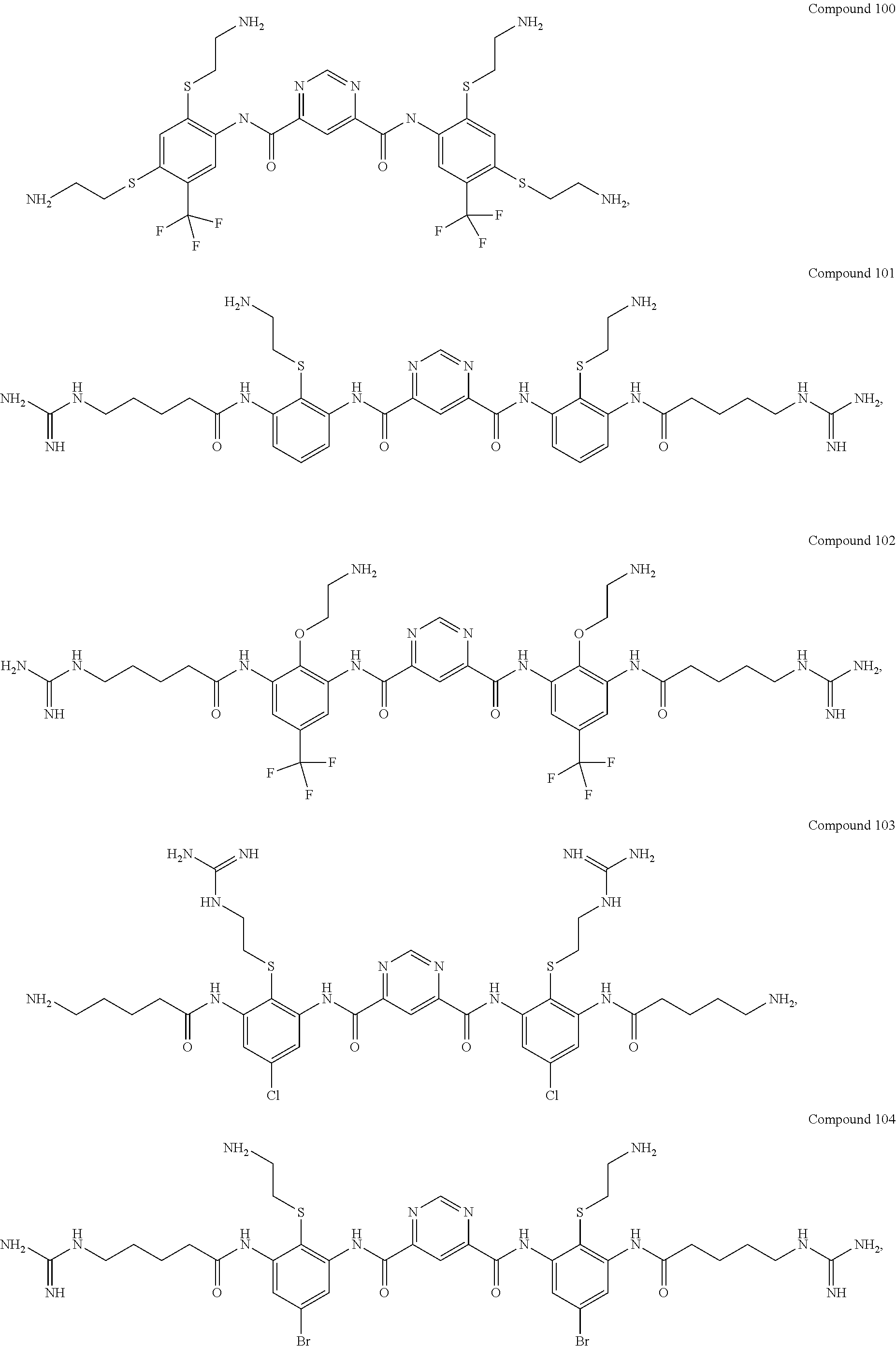

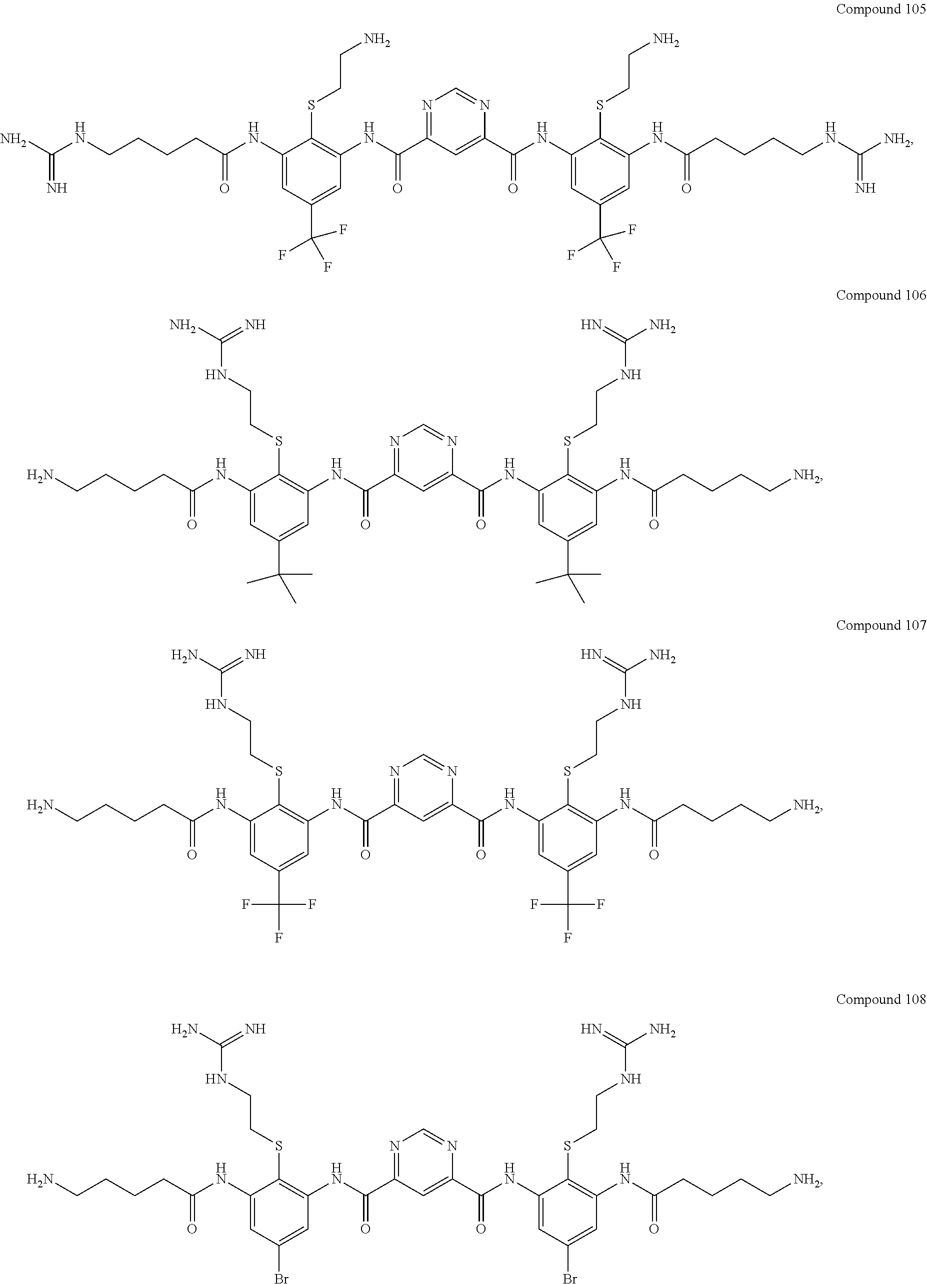 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3SCCN)ncn2)c1SCCN ; CC(C)(C)c1cc(NC(=O)CCCCN)c(SCCNC(=N)N)c(NC(=O)c2cc(C(=O)Nc3cc(C(C)(C)C)cc(NC(=O)CCCCN)c3SCCNC(=N)N)ncn2)c1 ; N=C(N)NCCSc1c(NC(=O)CCCCN)cc(C(F)(F)F)cc1NC(=O)c1cc(C(=O)Nc2cc(C(F)(F)F)cc(NC(=O)CCCCN)c2SCCNC(=N)N)ncn1 ; N=C(N)NCCSc1c(NC(=O)CCCCN)cc(Br)cc1NC(=O)c1cc(C(=O)Nc2cc(Br)cc(NC(=O)CCCCN)c2SCCNC(=N)N)ncn1]

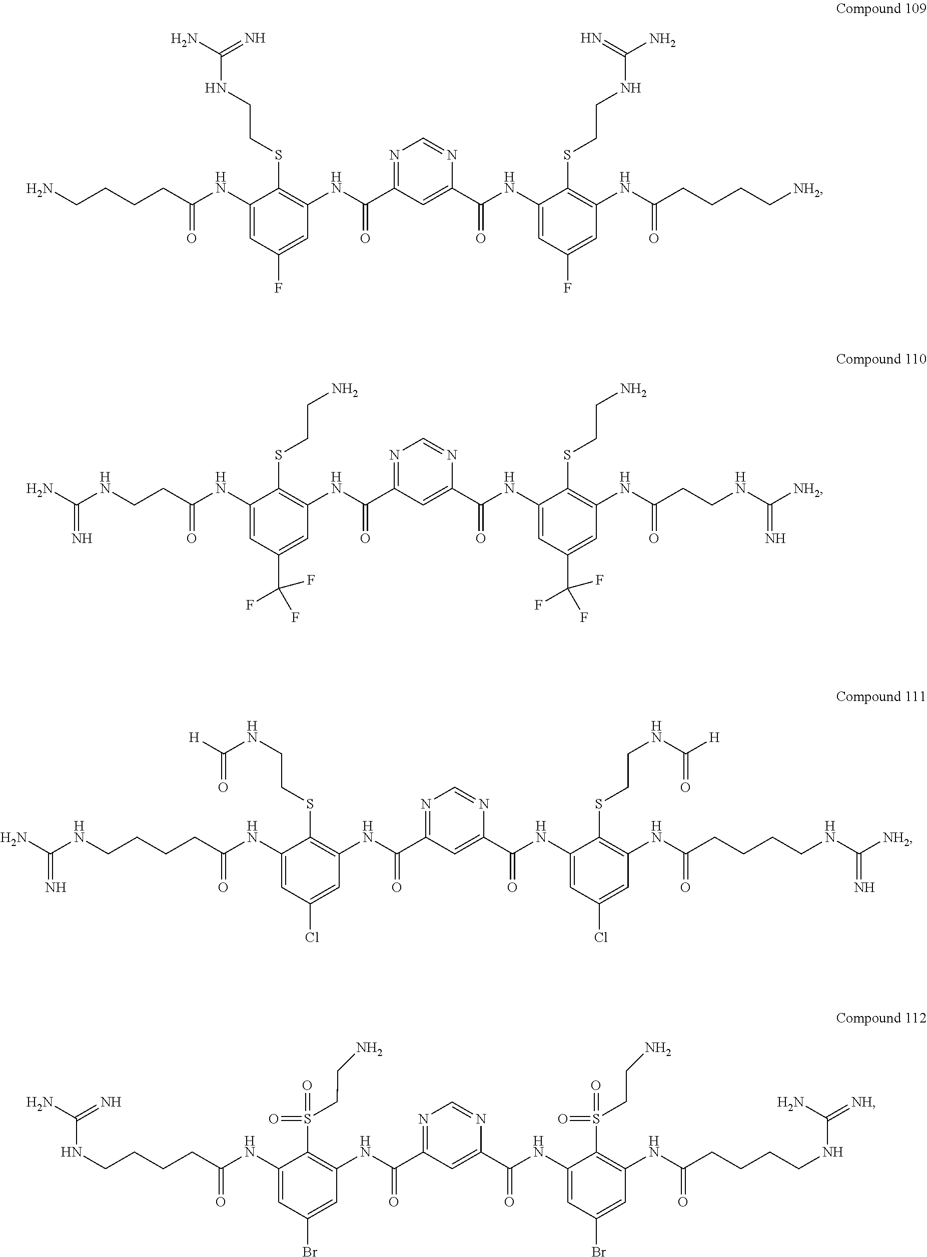 [SMILES: N=C(N)NCCSc1c(NC(=O)CCCCN)cc(F)cc1NC(=O)c1cc(C(=O)Nc2cc(F)cc(NC(=O)CCCCN)c2SCCNC(=N)N)ncn1 ; N=C(N)NCCC(=O)Nc1cc(C(F)(F)F)cc(NC(=O)c2cc(C(=O)Nc3cc(C(F)(F)F)cc(NC(=O)CCNC(=N)N)c3SCCN)ncn2)c1SCCN ; N=C(N)NCCCCC(=O)Nc1cc(Cl)cc(NC(=O)c2cc(C(=O)Nc3cc(Cl)cc(NC(=O)CCCCNC(=N)N)c3SCCNC=O)ncn2)c1SCCNC=O ; N=C(N)NCCCCC(=O)Nc1cc(Br)cc(NC(=O)c2cc(C(=O)Nc3cc(Br)cc(NC(=O)CCCCNC(=N)N)c3S(=O)(=O)CCN)ncn2)c1S(=O)(=O)CCN]

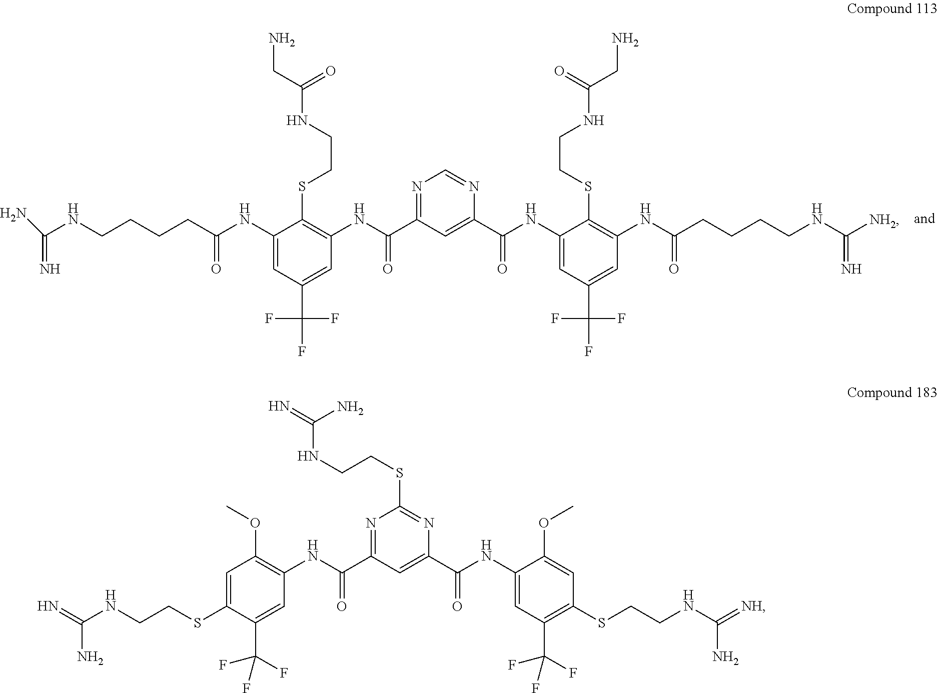 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3SCCNC(=O)CN)ncn2)c1SCCNC(=O)CN ; COc1cc(SCCNC(=N)N)c(C(F)(F)F)cc1NC(=O)c1cc(C(=O)Nc2cc(C(F)(F)F)c(SCCNC(=N)N)cc2OC)nc(SCCNC(=N)N)n1]

or a pharmaceutically acceptable salt thereof.

In any of the methods described herein, the compound of Formula I, Formula II, or Formula III comprises:

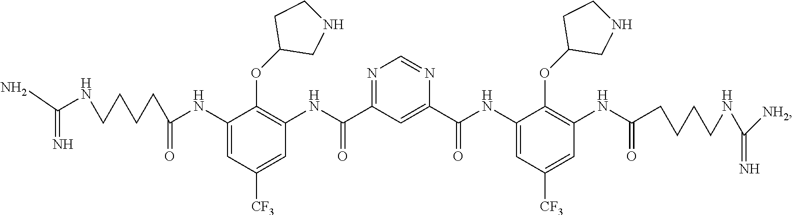 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3OC3CCNC3)ncn2)c1OC1CCNC1]

or a pharmaceutically acceptable salt thereof.

In any of the methods described herein, the compound of Formula I, Formula II, or Formula III comprises:

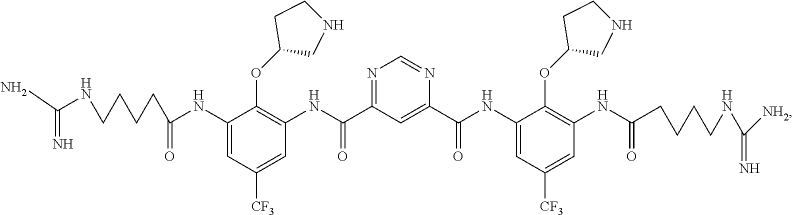 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3O[C@@H]3CCNC3)ncn2)c1O[C@@H]1CCNC1]

or a pharmaceutically acceptable salt thereof.

In any of the methods described herein, any one or more of the genuses, sub-genuses, or species of the above compounds may be excluded.

The compounds described herein can be prepared as described in the following patents and patent publications: US Published Patent Appl. Nos. US 2006-0041023 A₁, US 2004-0202639 A₁, US 2005-0287108 A₁, and US 2006-0024264 A₁, as well as U.S. Pat. No. 7,173,102. Examples of the design, synthesis, and testing of arylamide compounds are also presented in Tew et al., Proc. Natl. Acad. Sci. USA, 2002, 99, 5110-5114 and in WIPO Publication No. WO 2004/082634.

The compounds can be synthesized by solid-phase synthetic procedures well known to those of skill in the art. See, for example, Tew et al., Proc. Natl. Acad. Sci. USA, 2002, 99, 5110-5114; Barany et al., Int. J. Pept. Prot. Res., 1987, 30, 705-739; Solid-phase Synthesis: A Practical Guide, Kates, S. A., and Albericio, F., eds., Marcel Dekker, New York (2000); and Dörwald, F. Z., Organic Synthesis on Solid Phase: Supports, Linkers, Reactions, 2nd Ed., Wiley-VCH, Weinheim (2002).

The present disclosure also provides the combination of any one or more of the compounds of Formula I, Formula II, Formula III, or Formula IV, or a pharmaceutically acceptable salt thereof, and one or more other anti-fungal agents in a pharmaceutical composition. In some embodiments, the composition comprises any one or more of the compounds of Formula I, or a pharmaceutically acceptable salt thereof, and one or more other anti-fungal agent(s). In some embodiments, the composition comprises any one or more of the compounds of Formula II, or a pharmaceutically acceptable salt thereof, and one or more other anti-fungal agent(s). In some embodiments, the composition comprises any one or more of the compounds of Formula III, or a pharmaceutically acceptable salt thereof, and one or more other anti-fungal agent(s). In some embodiments, the composition comprises any one or more of the compounds of Formula IV, or a pharmaceutically acceptable salt thereof, and one or more other anti-fungal agent(s).

In some embodiments, the other anti-fungal agent is an azole or an echinocandin. In some embodiments, the other anti-fungal agent is an azole. In some embodiments, the azole is itraconazole, posaconazole, voriconazole (VOR), or isavuconazole. In some embodiments, the azole is itraconazole. In some embodiments, the azole is posaconazole. In some embodiments, the azole is voriconazole. In some embodiments, the azole is isavuconazole. In some embodiments, the other anti-fungal agent is an echinocandin. In some embodiments, the echinocandin is caspofungin (CAS). In some embodiments, the other anti-fungal agent is nystatin, miconazole, Gentian violet, or amphotericin B. In some embodiments, the other anti-fungal agent is nystatin. In some embodiments, the other anti-fungal agent is miconazole. In some embodiments, the other anti-fungal agent is Gentian violet. In some embodiments, the other anti-fungal agent is amphotericin B. Additional anti-fungal agents include, but are not limited to, fosmanogepix, ibrexafungerp, olorofim, opelconazole, and rezafungin. In some embodiments, the other anti-fungal agent is fosmanogepix. In some embodiments, the other anti-fungal agent is ibrexafungerp. In some embodiments, the other anti-fungal agent is olorofim. In some embodiments, the other anti-fungal agent is opelconazole. In some embodiments, the other anti-fungal agent is rezafungin. In some embodiments, the other anti-fungal agent is Nikkomycin Z. Other anti-fungal agents include VT-1129, VT-1161, VT-1598, PC1244, SUBA-ITC, CAMB, MGCD290, T-2307, and VL-2397. Additional anti-fungal agents are disclosed in, for example, PCT Publication No. WO 2021/247781.

In some embodiments, any one or more of the compounds of Formula I, Formula II, Formula III, or Formula IV, or a pharmaceutically acceptable salt thereof, can be combined with a Protein Kinase C inhibitor, such as chelerenthrine or calphostin C.

In some embodiments, the pharmaceutical composition comprises a compound comprising the formula:

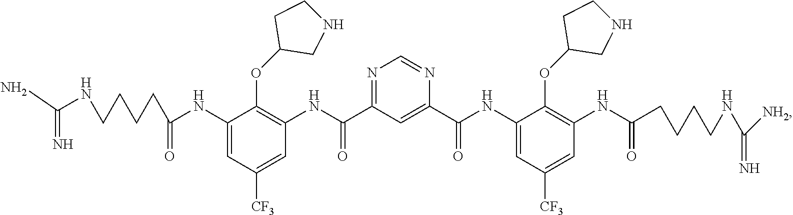 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3OC3CCNC3)ncn2)c1OC1CCNC1]

or a pharmaceutically acceptable salt thereof, and one or more other anti-fungal agent(s). In some embodiments, the pharmaceutical composition comprises a compound comprising the formula:

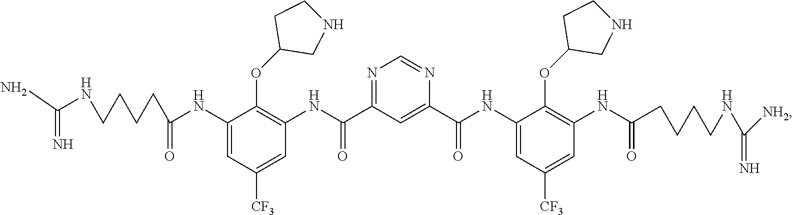 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3OC3CCNC3)ncn2)c1OC1CCNC1]

or a pharmaceutically acceptable salt thereof, and an azole. In some embodiments, the pharmaceutical composition comprises a compound comprising the formula:

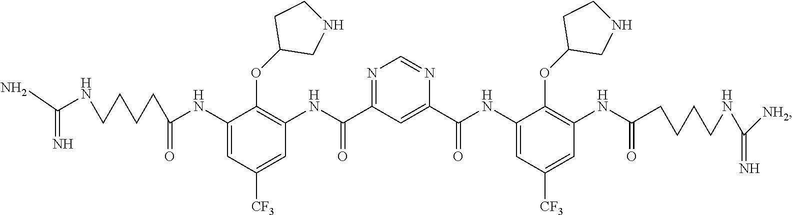 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3OC3CCNC3)ncn2)c1OC1CCNC1]

or a pharmaceutically acceptable salt thereof, and VOR. In some embodiments, the pharmaceutical composition comprises a compound comprising the formula:

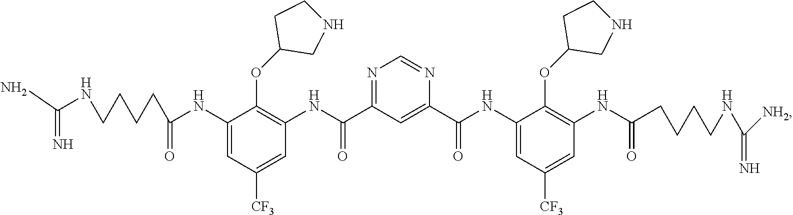 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3OC3CCNC3)ncn2)c1OC1CCNC1]

or a pharmaceutically acceptable salt thereof, and an echinocandin. In some embodiments, the pharmaceutical composition comprises a compound comprising the formula:

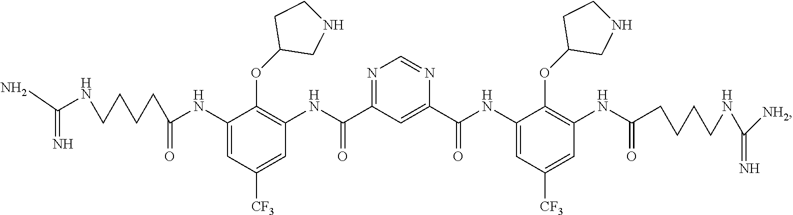 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3OC3CCNC3)ncn2)c1OC1CCNC1]

or a pharmaceutically acceptable salt thereof, and CAS.

In some embodiments, the pharmaceutical composition comprises a compound comprising the formula:

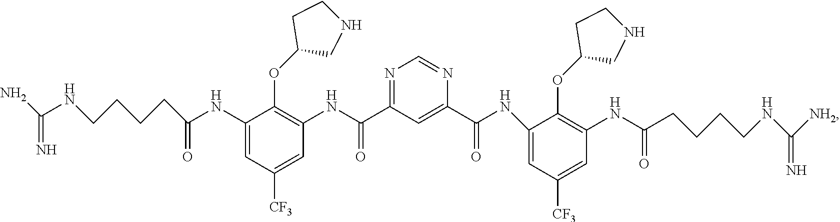 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3O[C@@H]3CCNC3)ncn2)c1O[C@@H]1CCNC1]

or a pharmaceutically acceptable salt thereof, and one or more other anti-fungal agent(s). In some embodiments, the pharmaceutical composition comprises a compound comprising the formula:

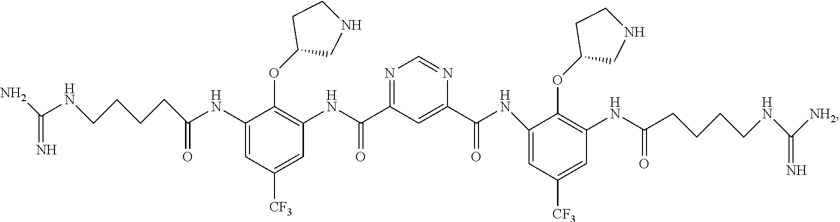 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3O[C@@H]3CCNC3)ncn2)c1O[C@@H]1CCNC1]

or a pharmaceutically acceptable salt thereof, and an azole. In some embodiments, the pharmaceutical composition comprises a compound comprising the formula:

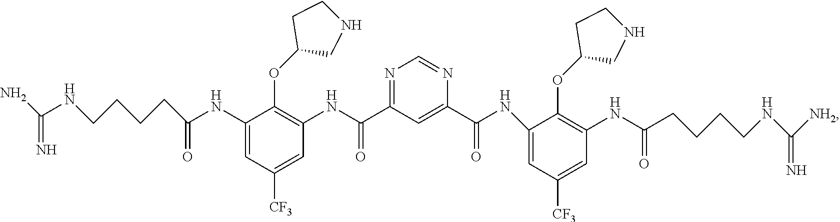 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3O[C@@H]3CCNC3)ncn2)c1O[C@@H]1CCNC1]

or a pharmaceutically acceptable salt thereof, and VOR. In some embodiments, the 10 pharmaceutical composition comprises a compound comprising the formula:

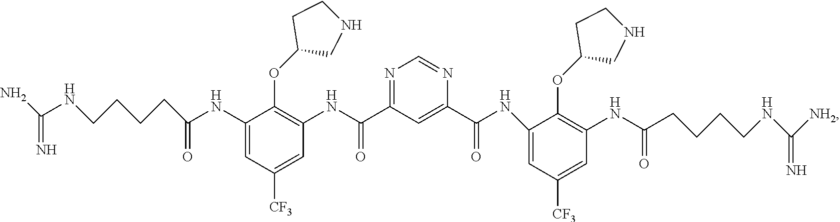 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3O[C@@H]3CCNC3)ncn2)c1O[C@@H]1CCNC1]

or a pharmaceutically acceptable salt thereof, and an echinocandin. In some embodiments, the pharmaceutical composition comprises a compound comprising the formula:

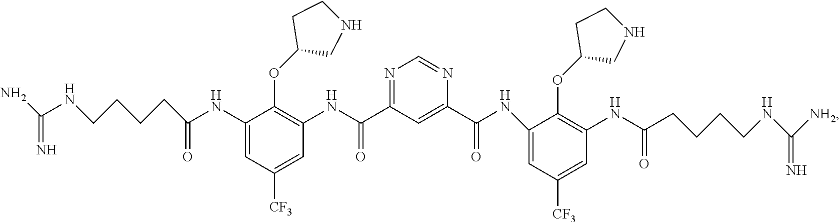 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3O[C@@H]3CCNC3)ncn2)c1O[C@@H]1CCNC1]

or a pharmaceutically acceptable salt thereof, and CAS.

The present disclosure also provides compositions comprising one or more of the compounds or salts described above and a pharmaceutically acceptable carrier.

In some embodiments, suitable dosage ranges for intravenous (i.v.) administration are 0.01 mg to 500 mg per kg body weight, 0.1 mg to 100 mg per kg body weight, 1 mg to 50 mg per kg body weight, or 10 mg to 35 mg per kg body weight. Suitable dosage ranges for other modes of administration can be calculated based on the forgoing dosages as known by those skilled in the art. For example, recommended dosages for intradermal, intramuscular, intraperitoneal, subcutaneous, epidural, sublingual, intracerebral, intravaginal, transdermal administration or administration by inhalation are in the range of 0.001 mg to 200 mg per kg of body weight, 0.01 mg to 100 mg per kg of body weight, 0.1 mg to 50 mg per kg of body weight, or 1 mg to 20 mg per kg of body weight. Effective doses may be extrapolated from dose-response curves derived from in vitro or animal model test systems. Such animal models and systems are well known in the art.

The compounds described herein can be administered in any conventional manner by any route where they are active. Administration can be systemic, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the compounds described herein (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams. The selection of the specific route of administration and the dose regimen is to be adjusted or titrated by the clinician according to methods known to the clinician to obtain the desired clinical response. The amount of compounds described herein to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician). The amount of a compound described herein that will be effective in the treatment and/or prevention of a fungal infection will depend on the nature of the fungal infection, and can be determined by standard clinical techniques. In addition, in vitro or in vivo assays may optionally be employed to help identify optimal dosage ranges. The precise dose to be employed in the compositions will also depend on the route of administration, and the seriousness of the fungal infection, and should be decided according to the judgment of the practitioner and each patient's circumstances. However, a suitable dosage range for oral administration is, generally, from about 0.001 milligram to about 200 milligrams per kilogram body weight. In some embodiments, the oral dose is from about 0.01 milligram to 100 milligrams per kilogram body weight, from about 0.01 milligram to about 70 milligrams per kilogram body weight, from about 0.1 milligram to about 50 milligrams per kilogram body weight, from 0.5 milligram to about 20 milligrams per kilogram body weight, or from about 1 milligram to about 10 milligrams per kilogram body weight. In some embodiments, the oral dose is about 5 milligrams per kilogram body weight.

The pharmaceutical compositions and/or formulations containing the compounds described herein and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a compound described herein. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance (see, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980)).

In some embodiments, the compounds described herein can be used with agents including, but not limited to, topical analgesics (e.g., lidocaine), barrier devices (e.g., GelClair), or rinses (e.g., Caphosol).

The compounds described herein can be formulated for parenteral administration by injection, such as by bolus injection or continuous infusion. The compounds described herein can be administered by continuous infusion subcutaneously over a period of about 15 minutes to about 24 hours. Formulations for injection can be presented in unit dosage form, such as in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compounds described herein can be formulated readily by combining these compounds with pharmaceutically acceptable carriers well known in the art. Such carriers enable the compounds described herein to be formulated as tablets, pills, dragees, capsules, liquids, gels, syrups, slurries, suspensions and the like, for oral ingestion by a patient to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration. For buccal administration, the compositions can take the form of, such as, tablets or lozenges formulated in a conventional manner.

For administration by inhalation, the compounds described herein can be delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, such as gelatin for use in an inhaler or insufflator can be formulated containing a powder mix of the compound and a suitable powder base such as lactose or starch.

The compounds described herein can also be formulated in rectal compositions such as suppositories or retention enemas, such as containing conventional suppository bases such as cocoa butter or other glycerides.

In transdermal administration, the compounds described herein, for example, can be applied to a plaster, or can be applied by transdermal, therapeutic systems that are consequently supplied to the organism.

The pharmaceutical compositions of the compounds described herein also can comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

A remarkable observation in the examples described herein is that BRI can potentiate CAS activity not only against A. fumigatus, but also C. albicans, C. auris, and C. neoformans. There are very few therapeutical options against the treatment of cryptococcosis and BRI demonstrated activity against C. neoformans in low concentrations (MIC=2.5 μM). C. neoformans is intrinsically resistant to CAS and only very high non-physiological CAS concentrations can partially inhibit C. neoformans growth. However, C. neoformans β-1,3-glucan synthase is very sensitive to CAS, which suggests that other mechanisms unrelated to β-1,3-glucan synthase resistance are important for CAS resistance.

The following examples will serve to further typify the nature of this invention but should not be construed as a limitation in the scope thereof, which scope is defined solely by the appended claims. In order that the invention disclosed herein may be more efficiently understood, examples are provided below. It should be understood that these examples are for illustrative purposes only and are not to be construed as limiting the invention in any manner.

### EXAMPLES

### Materials and Methods

Strains, media and cultivation methods: The A. fumigatus. Candida spp., and C. neoformans strains used in Examples 1 through 5 are listed in Table 1.

TABLE 1

| | Genotype | Reference |
|---|---|---|
| Aspergillus fumigatus strains | | |
| CEA17 (A1160) | akuB (KU80)Δ; pyrG1 MAT1-1 | 1 |
| ΔpphA | ΔpphA::pyrG, akuB (KU80)Δ, MAT1-1 | 2 |
| ΔsitA | ΔsitA::pyrG, akuB (KU80)Δ, MAT1-1 | 2 |
| ΔppzA | ΔppzA::pyrG, akuB (KU80)Δ, MAT1-1 | 2 |
| ΔpptA | ΔpptA::pyrG, akuB (KU80)Δ, MAT1-1 | 2 |
| ΔptcA | ΔptcA::pyrG, akuB (KU80)Δ, MAT1-1 | 2 |
| ΔptcB | ΔptcB::pyrG, akuB (KU80)Δ, MAT1-1 | 2 |
| ΔppmA | ΔppmA::pyrG, akuB (KU80)Δ, MAT1-1 | 2 |
| ΔptcD | ΔptcD::pyrG, akuB (KU80)Δ, MAT1-1 | 2 |
| ΔptcE | ΔptcE::pyrG, akuB (KU80)Δ, MAT1-1 | 2 |
| ΔptcF | ΔptcF::pyrG, akuB (KU80)Δ, MAT1-1 | 2 |
| ΔptcG | ΔptcG::pyrG, akuB (KU80)Δ, MAT1-1 | 2 |
| ΔptcH | ΔptcH::pyrG, akuB (KU80)Δ, MAT1-1 | 2 |
| ΔnemA | ΔnemA::pyrG, akuB (KU80)Δ, MAT1-1 | 2 |
| ΔpsrA | ΔpsrA::pyrG, akuB (KU80)Δ, MAT1-1 | 2 |
| ΔppsA | ΔppsA::pyrG, akuB (KU80)Δ, MAT1-1 | 2 |
| ΔdspD | ΔdspD::pyrG, akuB (KU80)Δ, MAT1-1 | 2 |
| ΔcdcA | ΔcdcA::pyrG, akuB (KU80)Δ, MAT1-1 | 2 |
| ΔdspA | ΔdspA::pyrG, akuB (KU80)Δ, MAT1-1 | 2 |
| ΔdspB | ΔdspB::pyrG, akuB (KU80)Δ, MAT1-1 | 2 |
| ΔptpB | ΔptpB::pyrG, akuB (KU80)Δ, MAT1-1 | 2 |
| ΔpypA | ΔpypA::pyrG, akuB (KU80)Δ, MAT1-1 | 2 |
| ΔltpA | ΔltpA::pyrG, akuB (KU80)Δ, MAT1-1 | 2 |
| ΔyphA | ΔyphA::pyrG, akuB (KU80)Δ, MAT1-1 | 2 |
| ΔptyA | ΔptyA::pyrG, akuB (KU80)Δ, MAT1-1 | 2 |
| ΔcalA | ΔcalA::pyrG, akuB (KU80)Δ, MAT1-1 | 2 |
| ΔmpkC | ΔmpkC::prtA | 3 |
| ΔsakA | ΔsakA::hph | 3 |
| ΔmpkA | ΔmpkA::hph | 4 |
| ΔmpkB | ΔakuBmpkB::ptrA; prtA+ | 5 |
| ΔsakA; ΔmpkC | ΔmpkC::prtA ΔsakA::hph | 6 |
| Afs35 (A1159) | akuA::loxP | 7 |
| CYP-15-75 | clinical isolate | 8 |
| CYP-15-108 | clinical isolate | 8 |
| CYP-15-109 | clinical isolate | 8 |
| CYP-15-147 | clinical isolate | 8 |
| 20089320 | clinical isolate | 8 |
| CYP-15-184 | clinical isolate | Katrien |
| | | Lagrou lab |
| CYP-15-190 | clinical isolate | Katrien |
| | | Lagrou lab |
| CYP-15-192 | clinical isolate | Katrien |
| | | Lagrou lab |
| CYP-15-195 | clinical isolate | Katrien |
| | | Lagrou lab |
| CYP-15-202 | clinical isolate | Katrien |
| | | Lagrou lab |
| CYP-15-212 | clinical isolate | Katrien |
| | | Lagrou lab |
| CYP-15-213 | clinical isolate | Katrien |
| | | Lagrou lab |
| CYP-15-215 | clinical isolate | Katrien |
| | | Lagrou lab |
| CYP-15-220 | clinical isolate | Katrien |
| | | Lagrou lab |
| CYP-15-221 | clinical isolate | Katrien |
| | | Lagrou lab |
| CYP-15-222 | clinical isolate | Katrien |
| | | Lagrou lab |
| CYP-15-224 | clinical isolate | Katrien |
| | | Lagrou lab |
| CYP-15-225 | clinical isolate | Katrien |
| | | Lagrou lab |
| CYP-15-226 | clinical isolate | Katrien |
| | | Lagrou lab |
| CYP-15-228 | clinical isolate | Katrien |
| | | Lagrou lab |
| CYP-15-229 | clinical isolate | Katrien |
| | | Lagrou lab |
| CYP-15-230 | clinical isolate | Katrien |
| | | Lagrou lab |
| CYP-15-231 | clinical isolate | Katrien |
| | | Lagrou lab |
| F14946 | clinical isolate | Gustavo |
| | | Goldman lab |
| CM7555 | clinical isolate | Gustavo |
| | | Goldman lab |
| DPL1033 | clinical isolate | David Perlin |
| | | lab |
| MD24053 | clinical isolate | David Perlin |
| | | lab |
| Candida spp. strains | | |
| C. auris 467/2015 | clinical isolate | Arnaldo |
| | | Colombo lab |
| C. auris 468/2015 | clinical isolate | Arnaldo |
| | | Colombo lab |
| C. auris 469/2015 | clinical isolate | Arnaldo |
| | | Colombo lab |
| C. auris 470/2015 | clinical isolate | Arnaldo |
| | | Colombo lab |
| C. auris 474/2015 | clinical isolate | Arnaldo |
| | | Colombo lab |
| C. albicans DPL1006 | clinical isolate; CAS^{R} | David Perlin |
| | | lab |
| C. albicans DPL1007 | clinical isolate; CAS^{R} | David Perlin |
| | | lab |
| C. albicans DPL1009 | clinical isolate; CAS^{R} | David Perlin |
| | | lab |
| C. albicans DPL1010 | clinical isolate; CAS^{R} | David Perlin |
| | | lab |
| C. albicans DPL1011 | clinical isolate; CAS^{R} | David Perlin |
| | | lab |
| Cryptococcus neoformans | | |
| H99 (ATCC 208821) | wild-type | ATCC |
| | | database |

1. Da Silva Ferreira et al., Eukaryot. Cell, 2006, 5, 207-211.
2. Winkelströter et al., G3 (Bethesda), 2015, 5, 1525-39.
3. Hagiwara et al., Fungal Genet. Biol., 2014, 73, 138-149.
4. Valiante et al., Fungal Genet. Biol., 2009, 46, 909-918.
5. Manfiolli et al., mBio, 2019, 10, e00215-19.
6. Bruder Nascimento et al., Mol. Microbiol., 2016, 100, 841-859.
7. FGSC, Fungal Genetic stock center.
8. Bastos et al., Front. Cell. Infect. Microbiol., 2019, 9, 414.

Aspergillus strains were grown in minimal medium (MM: 1% (wt/vol) glucose, 50 mL of 20× salt solution, trace elements, 2% (wt/vol), pH 6.5. For solid minimal medium 2% agar was added) at 37° C. Solutions of trace elements and salt solution are described by Käfer (Käfer, Adv. Genet., 1977, 19, 33-131). For the animal studies, A. fumigatus strain (wild-type, Δku80 pyrG⁺) was grown on MM. Fresh conidia were harvested in PBS and filtered through a Miracloth (Calbiochem). Conidial suspensions were spun for 5 minutes at 3,000× g, washed with PBS, counted using a hemocytometer and resuspended at a concentration of 5.0×10⁷ conidia/ml. Candida spp. and C. neoformans strains were grown and maintained on YPD (1% yeast extract, 2% peptone and 2% glucose). The plates were incubated at 37° C. for 4-7 days. Spores were collected in endotoxin-free Dulbecco phosphate buffered saline (PBS) containing 0.2% Tween 80 for Aspergillus, respectively. Collected spores were washed with PBS, and counted with a hemocytometer to prepare the final inocula.

Library drug screenings: The Pandemic Response box (400 compounds) and COVID box (160 compounds) (both available at the world wide web at “mmv.org”), the National Institutes of Health (NIH) clinical collection (727 compounds) (available at world wide web at “pubchem.ncbi.nlm.nih.gov/source/NIH%20Clinical%20Collection”) and the epigenetic probe library (115 compounds) (available at world wide web at “sgc-ffm.uni-frankfurt.de/”), totalizing 1,402 compounds, were screened in the current study. The primary screening was performed against the A. fumigatus wild-type strain by using the chemical libraries diluted in dimethyl sulfoxide (DMSO). The ability of each compound in combination (or not) with caspofungin (CAS) in blocking the fungal growth was visually determined. Briefly, each well of a flat-bottom polystyrene microplate was filled with 198 μL of liquid MM containing 1×10⁴ conidia/mL of A. fumigatus (wild-type strain). Subsequently 20 μM of each chemical compound was added in combination (or not) with 0.2 μg/mL of CAS to each well. This concentration represents the MEC for CAS against A. fumigatus. Plates were statically incubated for 48 hours at 37° C. Wells containing only medium, CAS (0.2 μg/mL) or DMSO were used as controls. Compounds presenting over 80% of visual fungal growth inhibition (in combination or not with CAS) were selected for further studies. All experiments were done in triplicate.

Alamar blue assays: The inhibition of the metabolic activity of A. fumigatus triggered by the drugs selected in the first screening was assessed by using Alamar blue (Life Technologies) according to Yamaguchi (Yamaguchi et al., J. Infect. Chemother., 2002, 8, 374-377). The experiment was performed by inoculation of 100 μL of liquid MM containing 2.5×10³ conidia/mL of the A. fumigatus wild-type strain supplemented or not with CAS (0.2 μg/mL) plus increasing concentration of each selected drug (0.6 to 20 μM) and 10% Alamar blue in 96-well plates. As positive controls, the drugs were replaced by the same volume of the medium. As the negative control, wells were filled with 90 μL of liquid MM plus 10 μL of Alamar Blue. Plates were incubated for 48 hours at 37° C. without shaking and results were read spectrophotometically by fluorescence (570) nm excitation−590 nm emission) in a microplate reader (SpectraMax® Paradigm® Multi-Mode Microplate Reader; Molecular Devices). Enhancers were defined as compounds that alone inhibited over 30% of A. fumigatus metabolic activity but in combination with CAS inhibited even more, while synergizers were defined as compounds which alone inhibited less than 30% of the fungal metabolic activity but in combination with CAS inhibited more than 30%.

A protein kinase inhibitors (PKI) library was also screened in combination with BRI. In total, 58 PKI were analyzed. Briefly, 100 μL of liquid MM containing 2.5×10³ conidia/mL of the A. fumigatus wild-type strain plus 10% alamar blue was inoculated with increasing concentration of PKI (5-80 μM) in the presence (or not) of BRI (20μM) and incubated 48 hours at 37° C. without shaking. To analyze the sensitivity of the null mutants ΔcalA, ΔmpkA and their complementing strains to BRI, 100 μL of liquid MM containing 2.5×10³ conidia/mL of each strain was inoculated in the presence (or not) of BRI (20 μM) plus 10% alamar blue and incubated 48 hours at 37° C. without shaking. To check if cyclosporine (CsA), chelerenthrine and/or calphostin C synergize with BRI, variable concentrations of each one of these drugs was analyzed in the presence of BRI (20 μM). A total 100 μL of liquid MM containing 2.5×103 conidia/mL of the A. fumigatus wild-type strain plus 10% alamar blue was inoculated with increasing concentration of CsA (25-200 μg/mL), chelerenthrine (0.78-6.25 μg/mL) and calphostin C (6.25-50 μg/mL) in the presence (or not) of BRI (20 μM). Plates were incubated for 48 hours at 37° C. without shaking. All experiments containing alamar blue were, after 48 hours incubation, read spectrophotometically by fluorescence and analyzed as previously described. Experiments were repeated at least three times.

Minimal inhibitory concentration (MIC): The BRI drug used for MIC assays was solubilized in DMSO. The minimal inhibitory concentration (MIC) of BRI for A. fumigatus was determined based on the M38-A₂ protocol of the Clinical and Laboratory Standards Institute (CLSI 2008) and for yeasts using M27-A3 method (CLSI, 2017). In brief, the MIC assay was performed in 96-well flat-bottom polystyrene microplate where 200 μL of a suspension (1×10⁴ conidia/mL) prepared in liquid MM was dispensed in each well and supplemented with increasing concentration of BRI (ranging from 0 to 160 μM). Plates were incubated at 37° C. without shaking for 48 hours and the inhibition of growth was evaluated. The MIC was defined as the lowest drug concentration that visually attained 100% of fungal growth inhibition compared with the control well. Wells containing only MM and DMSO were used as a control. Similar protocol was used for yeast organisms, except use of RPMI-1640, 1×10³ cells/mL/well and incubation of the plates for 48 hours (Candida spp.) or 72 hours (C. neoformans).

Combination of Brilacidin and caspofungin against yeasts: For measuring the effect of the combination BRI+CAS against yeast fungal pathogens, two methods were used: (i) metabolic activity by XTT-assay and (ii) colony forming units (CFUs). For the first method, C. neoformans, C. albicans, and C. auris 10⁴ cells were inoculated in RPMI-1640 supplemented with CAS 0 to 32 μg/ml (for C. neoformans) and 0 to 1 μg/ml (for Candida spp.) or the same concentrations of CAS combined with BRI 0.625 μM (for C. neoformans), BRI 20 μM (for C. albicans), and BRI 10 μM (for C. auris). After 48 hours of incubation, the viable cells were revealed using XTT-assay as described by Bastos (Bastos et al., Front. Cell. Infect. Microbiol., 2019, 9, 414). XTT-assays were also used for C. albicans caspofungin resistant strains but with CAS 0.5 μg/ml combined with BRI 5, 10 or 20 μM. The same experimental design was used for the CFUs determination, except that after 48 hours the cells present in the wells were plated on YPD (yeast extract 10g, peptone 20g, dextrose 20g, agar 20 g, water 1000 mL) and the plates were incubated at 30° C. for 24-48 hours for determining the survival percentage. The results are the average of three repetitions and are expressed as average+standard deviation.

Conidial viability exposed to brilacidin (BRI). voriconazole (VOR) and caspofungin (CAS): The viability of A. fumigatus conidia exposed to CAS+BRI or VOR+BRI was assessed by plating the cells after being treated. Initially, a suspension containing 1×10⁴ conidia/mL of A. fumigatus cells was prepared in liquid MM and 200μL of this suspension was dispensed in each well of a 96-well polystyrene microplate supplemented with CAS (0.2 or 0.50 μg/mL)+BRI (20 μM) or VOR (0.125 or 0.25 μg/mL)+BRI (20 μM). After 48 hours incubation at 37° C., a total of 100 conidia was plated in solid complete medium (YAG) (2% (w/v) glucose, 0.5% (w/v) yeast extract, trace elements) or minimal medium (1% (w/v) glucose, nitrate salts, trace elements, pH 6.5) and let to grow at 37° C. for 36 hours. The number of viable colonies was determined by counting the number of colony-forming unit (CFU) and expressed in comparison with the negative control (no germinated and untreated conidia), which gives 100% survival. Results are expressed as means and standard deviations (SD) from three independent experiments.

Biofilm assay: To test the susceptibility of pre-formed A. fumigatus biofilms to

VOR, CAS and to the combination of CAS+BRI and VOR+BRI, a suspension containing 10⁶ conidia per mL of the wild-type strain (Δku80 pyrG⁺) was prepared in liquid MM and 100 μL of it was inoculated in each well of a 96-well plate. After 24 hours of incubation at 37° C., 50 μL of fresh MM containing CAS, VOR or the combination of VOR and CAS with BRI was added to the biofilm to reach the final concentration as indicated and incubated for a further 12 hours at 37° C. Wells containing untreated conidia were used as a positive control. After, the metabolic activity of the cells was evaluated by adding 50 μL of an aqueous XTT solution (1 mg/mL of XTT and 125 μM of menadione) to each well. The plate was incubated for additional 1 hour at 37° C., centrifuged (2000 rpm, 5 minutes) and 100 μL of the supernatant was transferred to a flat-bottomed 96-well plate. The absorbance was measured at 450 nm on a plate reader (Synergy HTX Multi-Mode Reader- BioTek Instruments). The XTT assay's were performed in six replicates.

Phosphatase and kinase null mutant screening: An A. fumigatus phosphatase deletion library encompassing 25 null mutants for phosphatase catalytic subunits (Winkelströter et al., G3 (Bethesda), 2015, 5, 1525-39) was screened for sensitivity to the combination of CAS+BRI. A. fumigatus null mutants for MAPK (ΔsakA, ΔmpkC, ΔsakA; ΔmkC, ΔmpkB and ΔmpkA) were also screened. The assay was performed in 96-well flat-bottom polystyrene microplate. In each well a total of 200 μL of liquid MM plus conidia from the different mutants (1×10⁴ conidia/mL) was incubated in the presence of BRI (20 μM). Plates were incubated at 37° C. without shaking for 48 hours and the inhibition of growth was visually evaluated. Wells containing only MM and DMSO were used as a control.

Membrane potential determination: The effect of the CAS (0.125 μg/mL), BRI (1 μM) or the combination CAS+BRI (0.125 μg/mL and 1 μM, respectively) on the cell membrane potential was assessed by using the bis-(1,3-dibutylbarbituric acid) trimethine oxonol-DiBAC4(3) reagent (Invitrogen, Carlsbad, CA, USA) according to Veerana (Veerana et al., Microb. Biotechnol., 2021, 14, 262-276) with modifications. A. fumigatus conidia were inoculated on coverslips in 5 mL of liquid MM and cultivated for 16 hours at 30° C. Further, coverslips containing adherent germlings were left untreated or treated with CAS, BRI or CAS+BRI plus 3 μg/ml DIBAC+(3) and incubated for 30 minutes at 30° C. in the dark. After, the germlings were washed with sterile PBS (140 mM NaCl, 2 mM KCl, 10 mM NaHPO₄, 1.8 mM KH₂PO₄, pH 7.4). The fluorescence was analyzed with excitation wavelength of 450-490 nm, and emission wavelength of 525-550 nm on the Observer Z1 fluorescence microscope (Carl Zeiss) using the 100× with Differential interference contrast (DIC) images. Fluorescent images were captured with an AxioCam camera (Carl Zeiss, Inc.) and processed using the Axio Vision software (version 4.8). In each experiment, at least 50 germlings were counted and the experiment repeated at least 3 three times.

Cytotoxicity assay: Cytotoxicity assays in A549 human lung cancer cells were performed using XTT assay as indicated in the manufacturers' instructions. Cells (2×10⁵ cells/well) were seeded in 96-well tissue plates and incubated in Dulbecco's Modified Eagle Medium (DMEM) culture medium. After 24 hours of incubation, the cells were treated with BRI (40 and 80 μM/well), CAS (50, 100 and 200 μg/well) or in different CAS+BRI combinations. After 48 hours incubation, the cell viability was assessed by using the XTT kit (Roche Applied Science) according to the manufacturer's instructions. Formazan formation was quantified spectrophotometrically at 450 nm (reference wavelength 620 nm) using a microplate reader. The experiment was made in three replicates. Viability was calculated using the background-corrected absorbance as follows: Cell viability (%)=absorbance value of experiment well/absorbance value of control well×100.

Killing assay: The type II pneumocyte cell line A549 was cultured using DMEM (ThermoFischer Scientific, Paisley. UK) supplemented with 10% fetal bovine serum (FBS) and 1% penicillin-streptomycin (Sigma-Aldrich, Gillingham, UK) and seeded at a density of 10⁶ cells/ml in 24-well plates (Corning). The cells were treated with Brilacidin (20, 40 and 80 μM/well), Caspofungin (100 μg/well) or in different combinations between them and challenged with A. fumigatus conidia at a multiplicity of infection of 1:10. After 24 hours of incubation in 5% CO₂ the culture media was removed, and 2 ml of sterile water was added to the wells. A P1000 tip was then used to scrape away the cell monolayer and the cell suspension was collected. This suspension was then diluted 1:1000 and 100 μl was plated on Sabouraud Dextrose Agar Media before the plates were incubated a 37° C. overnight. The numbers of CFUs were determined after 24 hours of growth. A volume of 50 μl of the inoculum adjusted to 103/ml was also plated on SAB agar to correct CFU counts. The CFU percentage for each sample was calculated and the results were plotted using Graphpad Prism (GraphPad Software, Inc., La Jolla, CA, USA). A p value ≤0.001 was considered significant.

Fungal burden: Inbred female mice (BALB/c strain; body weight, 20-22 g) were housed in vented cages containing five animals. Mice were immunosuppressed with cyclophosphamide (150 mg per kg of body weight), which was administered intraperitoneally on days −4, −1 and 2 prior to and post infection (infection day is “day 0”). Hydrocortisonacetate (200 mg/kg body weight) was injected subcutaneously on day −3. Mice (5 mice per group) were anesthetized by halothane inhalation and infected by intranasal instillation of 20 μL of 1.0×106 conidia of A. fumigatus Δku80 pyrG⁺ (wild-type) (the viability of the administered inoculum was determined by incubating a serial dilution of the conidia on MM medium, at 37° C.). As a negative control, a group of 5 mice received PBS only. On the same day of infection (day 0), mice received concomitantly the first dose of treatment with BRI (50 mg per kg of body weight) and/or CAS (1 mg per kg of body weight), administered intraperitoneally. The second dose of drugs was administered 24 hours after infection. Animals were sacrificed 72 hours post-infection, and the lungs were harvested and immediately frozen in liquid nitrogen. Samples were lyophilized and homogenized by vortexing with glass beads for 5 minutes, and DNA was extracted via the phenol/chloroform method.

DNA quantity and quality were assessed using a NanoDrop 2000 spectrophotometer (Thermo Scientific). Quantitative real-time PCRs were performed using 400 ng of total DNA from each sample, and primers to amplify the 18S rRNA region of A. fumigatus and an intronic region of mouse GAPDH (glyceraldehyde-3-phosphate dehydrogenase). Six-point standard curves were calculated using serial dilutions of gDNA from A. fumigatus strain and the uninfected mouse lung. Fungal and mouse DNA quantities were obtained from the threshold cycle (Ct) values from an appropriate standard curve.

Statistical analysis: Grouped column plots with standard deviation error bars were used for representations of data. For comparisons with data from wild-type or control conditions, one-tailed, paired/tests or one-way analysis of variance (ANOVA) were performed. All statistical analyses and graphics building were performed by using GraphPad Prism 5.00 (GraphPad Software).

Fractional Inhibitory Concentration (FIC) index analysis, for Example 3: To determine synergy, additive, indifference, or antagonism between a conventional antifungal agent and BRI, the FIC index method (Meletiadis et al., Med Mycol., 2005, 43, 133-152) was used. Briefly, for all of the wells of the microtitration plates that corresponded to an MIC, the sum of the FICs (EFIC) was calculated for each well with the equation ΣFIC=FICA+FICB=(CA(comb)/MICA(alone))+(CB(comb)/MICB(alone)), where MICA(alone) and MICB(alone) are the MICs of drugs A and B when acting alone, and CA(comb) and CB(comb) are the concentrations of the drugs A and B at the iso-effective combinations. A FIC index of <0.5 indicates synergism, >0.5-1 indicates additive effects, >1 to <2 indifference, and ≥2 is considered to be antagonism (Faleiro and Miguel, In: Fighting Multidrug Resistant with Herbal Extracts Oils and Their components, Chapter 6, 2013, 20) Academic Press, San Diego, Editors Rai and Kon).

RNA extraction. RNA-sequencing. cDNA synthesis and RTqPCR: All experiments were carried out in biological triplicates and conidia (10⁷) were inoculated in liquid MM and A. fumigatus strains were grown for 16 hours at 37 ° C. and treated or not with different concentrations of BRI, CAS, or BRI+CAS for 1 hour. For total RNA isolation, mycelia were ground in liquid nitrogen and total RNA was extracted using TRIzol (Invitrogen), treated with RQ1 RNase-free DNase I (Promega), and purified using the RNAeasy kit (Qiagen) according to the manufacturer's instructions. RNA was quantified using a NanoDrop and Qubit fluorometer, and analyzed using an Agilent 2100 Bioanalyzer system to assess the integrity of the RNA. All RNA had an RNA integrity number (RIN) between 8.0 and 10 (Thermo Scientific) according to the manufacturer's protocol.

For RT-qPCR, the RNA was reverse transcribed to cDNA using the ImProm-II reverse transcription system (Promega) according to manufacturer's instructions, and the synthesized cDNA was used for real-time analysis using the SYBR green PCR master mix kit (Applied Biosystems) in the ABI 7500 Fast real-time PCR system (Applied Biosystems, Foster City, CA, USA). Primer sequences are on file.

Staining for chitin: This procedure was performed, as described by Winkelströter et al., G3 (Bethesda), 2015, 5, 1525-39; Graham et al., J. Immunol. Methods., 2006, 314, 164-169; Winkelströter et al., Mol. Microbiol., 2015, 96, 42-54. Briefly, A. fumigatus conidia (2×10⁴) were grown in 200 μL of MM for 16 hours at 37° C. The germlings were exposed or not to different concentrations of BRI, CAS or BRI+CAS for 4 hours and the culture medium was removed and the germlings were UV-irradiated (600,000 μJ). For chitin staining, 200 μL of a PBS solution with 10 μg/ml of CFW were added to UV-irradiated germlings, incubated for 5 minutes at room temperature, washed three times with PBS and fluorescence was read at 380 nm excitation and 450 nm emission. All the experiments were performed with 8 repetitions and fluorescence was read in a microtiter plate reader Synergy/HTX multi-mode reader (Biotek).

In vitro susceptibility (MIC) testing. for Examples 6 and 7 only: The BRI drug used for MIC assays was prepared with water (stock solution of 6.4 mg/ml). Macrobroth dilution was performed in 2 mL tubes, for MIC determinations as previously detailed (Denning et al., Diag Micro Infect Dis., 1992, 15, 21-34; Clinical and Laboratory Standards Institute (CLSI), 2017, M38 Edition 3; CLSI, 2017, M27 Edition 4). In brief, the initial inoculum was 10³ fungi (yeasts, conidia, hyphal fragments, etc.)/mL with a two-fold dilution range of drug, starting at 64 μg/ml (in double serial dilutions down to 0.25 μg/ml), and incubated at a temperature to approximate human body temperature (35-37° C.). The MIC values were determined when the control (drug-free) growth was 4+. Two MIC values were assigned to BRI: the 100% inhibition endpoint was defined as the first tube without growth, and the 50% inhibition endpoint as 2+ growth compared to the 4+ in the control.

Checkerboard Method of Drug Interaction testing (FIC index analysis). for Example 7 only: To evaluate the effect of two drugs in combination, a checkerboard arrangement was performed employing ˜60 tubes/test. It allows 2 drugs to be studied over a variety of concentrations of both: e.g., high concentrations of drug A with high, medium and low of drug B; medium concentrations of drug A with high, medium and low of drug B; low concentrations of drug A with high, medium and low of drug B; and vice versa for all for drug B. In the “outside” rows (bottom row of checkerboard for one drug, and left-most row of the other), there is no mixing of the drugs, enabling a reading of the MICs of each drug alone. Where those 2 rows intersect (the extreme lower-left corner of the matrix), there is no drug present, and this control growth is the measure against which every tube of the matrix is compared. The rest of the matrix is tubes with the various mixtures. The matrices are up to 9×9 combinations.

The concentration ranges tested were: brilacidin 0.25-64 μg/ml; fluconazole 0.25-32 μg/ml; posaconazole 0.125-16 μg/ml; amphotericin B 0.125-8 μg/ml; micafungin 0.031-16 μg/ml; flucytosine 0.125-64 μg/ml; and voriconazole 0.063-8 μg/ml. When the same microbe was tested in different checkerboards, the BRI MICs were determined independently in each.

The effect of two drugs in combination was calculated via a Fractional Inhibitory Concentration index (FICi), determined by the equation: FICi=(MIC_{A} in combination/MIC_{A} tested alone)+(MIC_{B} in combination/MIC_{B} tested alone), (Eliopoulos and Eliopoulos, Clin. Microbiol. Rev., 1988, 1, 139-156; and Sass et al., Pathogens, 2021, 10, 519). Drug interactions were classified as strong synergism when FICi <0.5; weak synergism when 0.5-<1; additive when 1-<2; indifferent when FICI =2; and antagonistic when FICi >2.

### Example 1: Screening of the COVID Box, Pandemic Response Box, NIH Clinical, and Epigenetic Compound Libraries

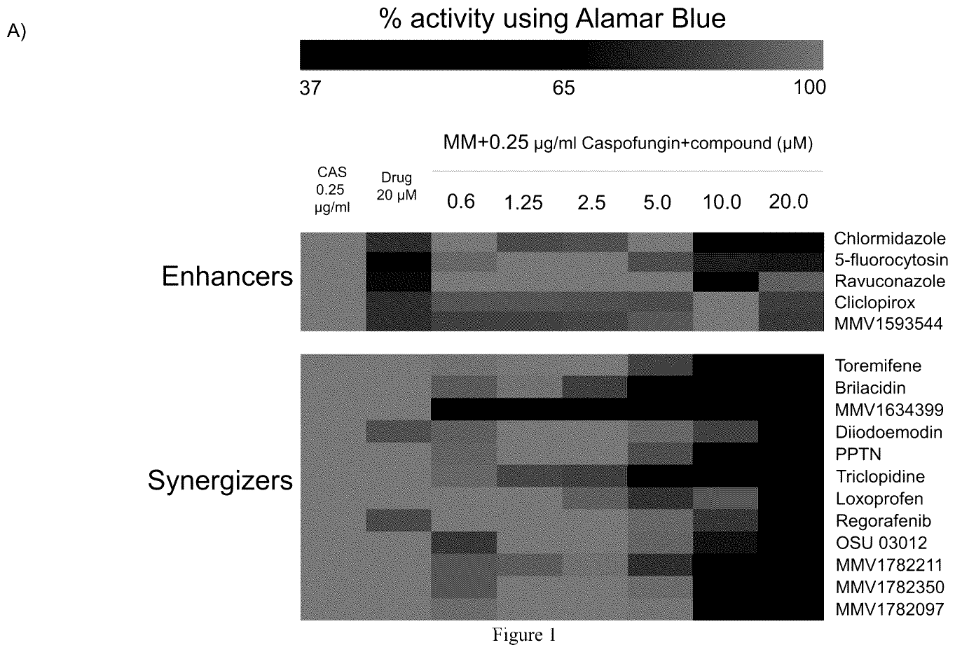

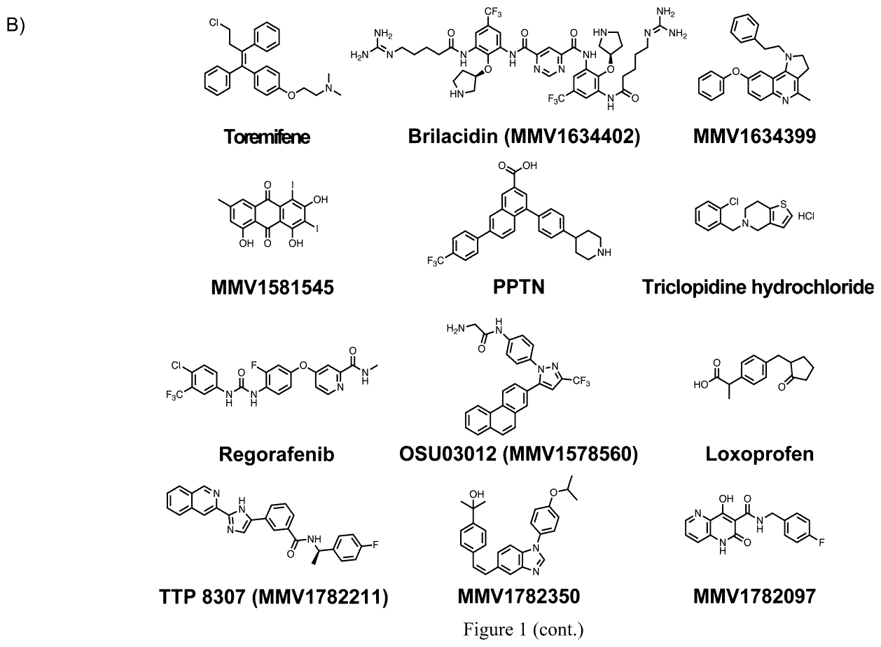

To identify compounds that can enhance or synergize with caspofungin (CAS) activity against A. fumigatus, the Minimal Effective Concentration (MEC) assay was used to screen the fungus susceptibility to four chemical drug libraries: i) the COVID Box (containing 160) compounds, see world wide web at “mmv.org/mmv-open/archived-projects/covid-box”), ii) the Pandemic Response Box (containing 400 compounds, see world wide web at “mmv.org/mmv-open/pandemic-response-box/about-pandemic-response-box”), iii) the National Institutes of Health (NIH) clinical collection (NCC) (containing 727 compounds; see world wide web at “pubchem.ncbi.nlm.nih.gov/source/NIH%20Clinical%20Collection”), and iv) the epigenetic probe library (containing 115 compounds, see world wide web at “sgc-ffm.uni-frankfurt.de/”). In total, combining all libraries, 1,402 compounds were assessed by using a combination of 0.2 μg/ml of CAS (a concentration below the minimum effective concentration, MEC; MEC of CAS is 0.25 μg/ml) and up to 20 μM of each compound compared to the effect on growth of A. fumigatus of each drug alone. First, growth was assessed by two independent rounds of visual inspection and 17 compounds were selected that could inhibit A. fumigatus growth. Second, A. fumigatus growth in the presence of CAS 0.2 μg/ml alone, each of these 17 compounds at 20 μM alone, and a combination of each of these compounds from 0.6 to 20 μM plus CAS 0.2 μg/ml was quantified by using Alamar blue (see, FIG. 1, Panel A). Based on this assay, enhancers were defined as the compounds that alone could inhibit over 30% of A. fumigatus metabolic activity but in combination with CAS inhibited even more, while synergizers were defined as compounds which alone inhibited less than 30% of the fungal metabolic activity but in combination with CAS inhibited more than 30%. Five compounds were classified as enhancers (FIG. 1, Panel A): i) chlormidazole (see, world wide web at “go.drugbank.com/drugs/DB13611”), ii) ravuconazole (see, world wide web at “go.drugbank.com/drugs/DB06440”; both inhibiting ergosterol biosynthesis), iii) 5-fluorocytosin (see, world wide web at “go.drugbank.com/unearth/q?c=_score&d=down&query=5-fluorocytosin&searcher=drugs”; that inhibits the RNA and DNA biosynthesis), iv) ciclopyxox (see, world wide web at “go.drugbank.com/unearth/q?utf8=%E2%9C%93&searcher=drugs&query=ciclopyxox”; it is thought to act through the chelation of polyvalent metal cations, such as Fe³⁺ and Al³⁺; Niewerth et al., Antimicrob. Agents Chemother., 2003, 47, 1805-17), and v) MMV¹⁵⁹³⁵⁴⁴ (a possible antiviral compound that inhibits SARS-COV-2 infection in vitro; Holwerda et al., Microorganisms, 2020, 8, 1872). Twelve compounds were classified as synergizers (FIG. 1, Panels A and B): i) toremifene (see, world wide web at “go.drugbank.com/drugs/DB00539”; a nonsteroidal triphenylethylene derivative used as an antitumor drug that appears to bind to the estrogen receptors competing with estradiol), ii) brilacidin (see, world wide web at “go.drugbank.com/drugs/DB12997”; a compound that acts as a mimetic of host defense peptides; Mensa et al., Antimicrob. Agents Chemother., 2014, 58, 5136-45), (iii) MMV¹⁶³⁴³⁹⁹ (a quinoline eficiente as anti-malarial; Reader et al., Nat. Commun., 2021, 12, 269), iv) Diiodoemodin or MMV¹⁵⁸¹⁵⁴⁵ (an anti-bacterial emodin derivative; Ji et al., Ann. Transl. Med., 2020, 8, 92), v) PPTN (a potent. high-affinity, competitive and highly selective nucleotide-sugar-activated P2Y14 receptor antagonist; Barrett et al., Mol. Pharmacol., 2013. 84. 41-9), vi) triclopidine (a prodrug that is metabolized to an active form. which blocks the ADP receptor that is involved in GPIIb/Illa receptor activation leading to platelet aggregation; see, world wide web at “go.drugbank.com/drugs/DB00208”), vii) loxoprofen (a non-steroidal anti-inflammatory drug that acts as a non-selective inhibitor of cyclooxygenase enzymes. which are responsible for the formation of various biologically active pain. fever. and inflammatory mediators; see, world wide web at “go.drugbank.com/drugs/DB09212”), viii) regorafenib (a small molecule inhibitor of multiple membrane-bound and intracellular kinases involved in normal cellular functions and in pathologic processes such as oncogenesis. tumor angiogenesis, and maintenance of the tumor microenvironment; see, world wide web at “go.drugbank.com/drugs/DB08896”), ix) OSU 03012 (a potent inhibitor of recombinant phosphoinositide-dependent kinase 1; Tseng et al., Blood, 2005, 105, 4021-7), x) MMV¹⁷⁸²²¹¹ (an inhibitor of the SARS-COV-2 main protease; see, world wide web at “chemrxiv.org/engage/chemrxiv/article-details/60c753ed469df403bef44e65”), xi) MMV¹⁷⁸²³⁵⁰, and xii) MMV¹⁷⁸²⁰⁹⁷ (two uncharacterized antivirals).

Taken together, these results suggest that many compounds were identified that can enhance or synergize the activity of CAS against A. fumigatus. The synergizers have very different mechanisms of action and targets, most of them apparently not conserved in A. fumigatus.

### Example 2: BRI Converts CAS into a Fungicidal Drug and Overcomes CAS-Resistance

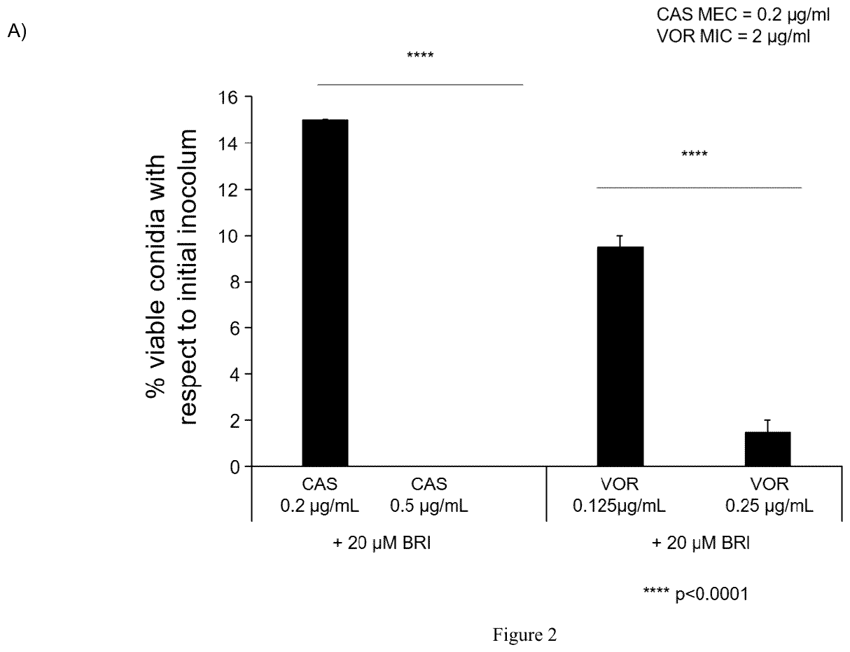

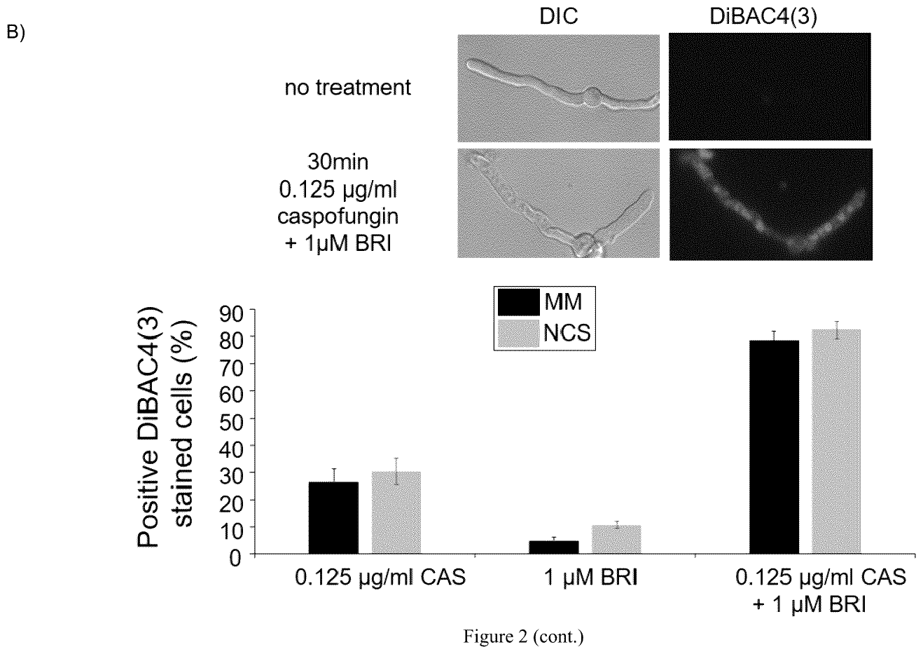

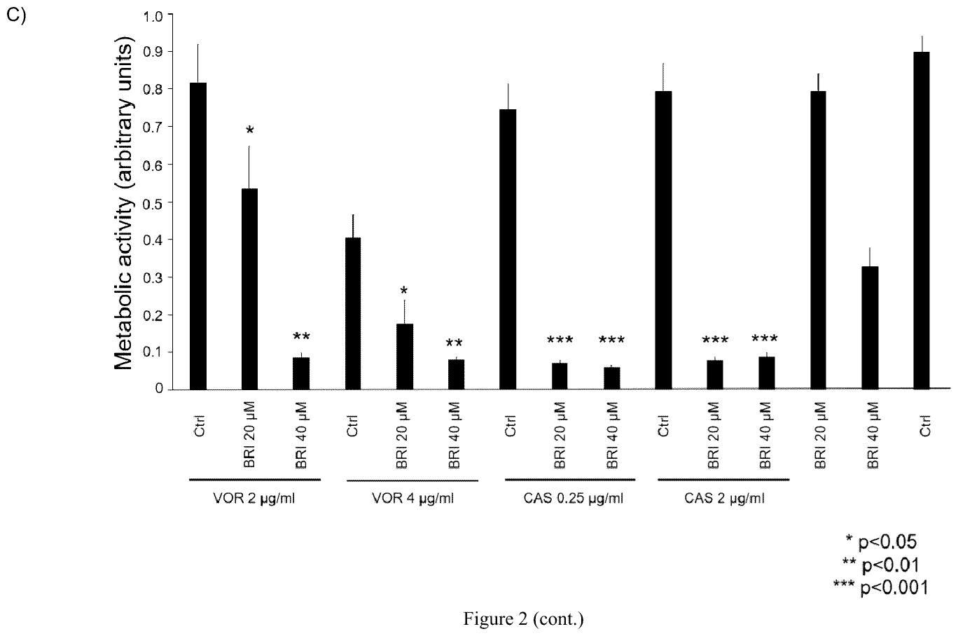

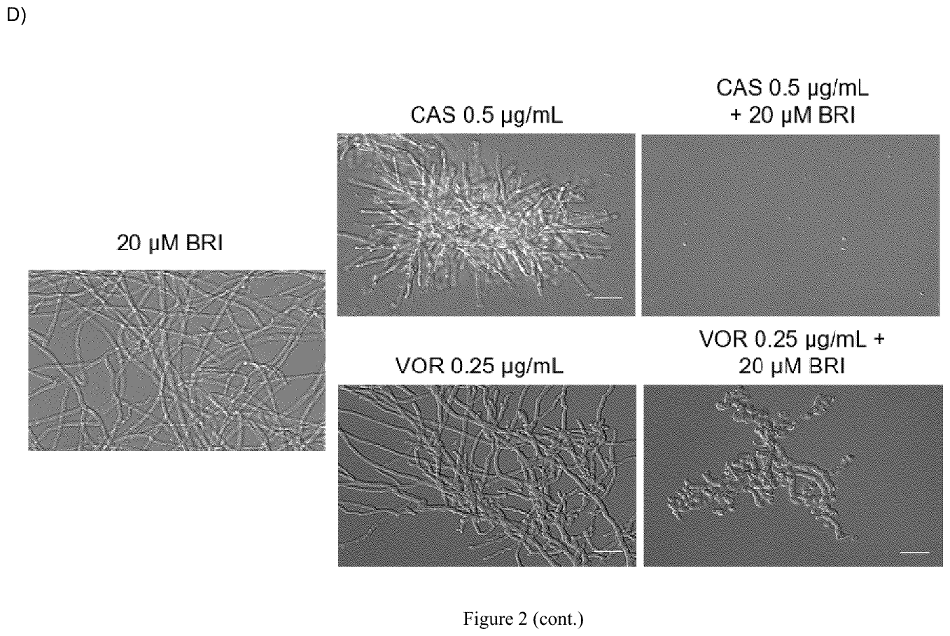

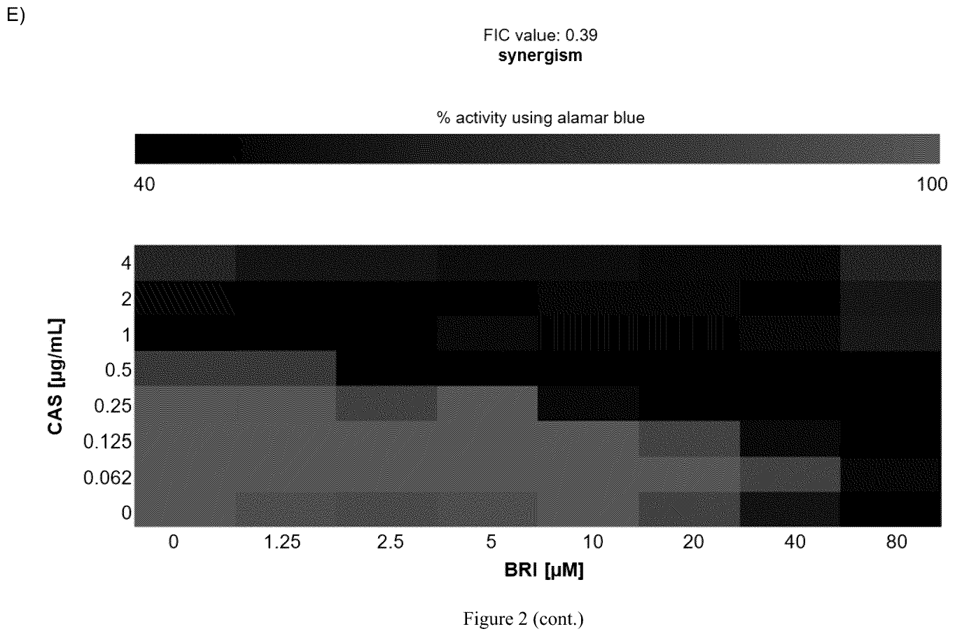

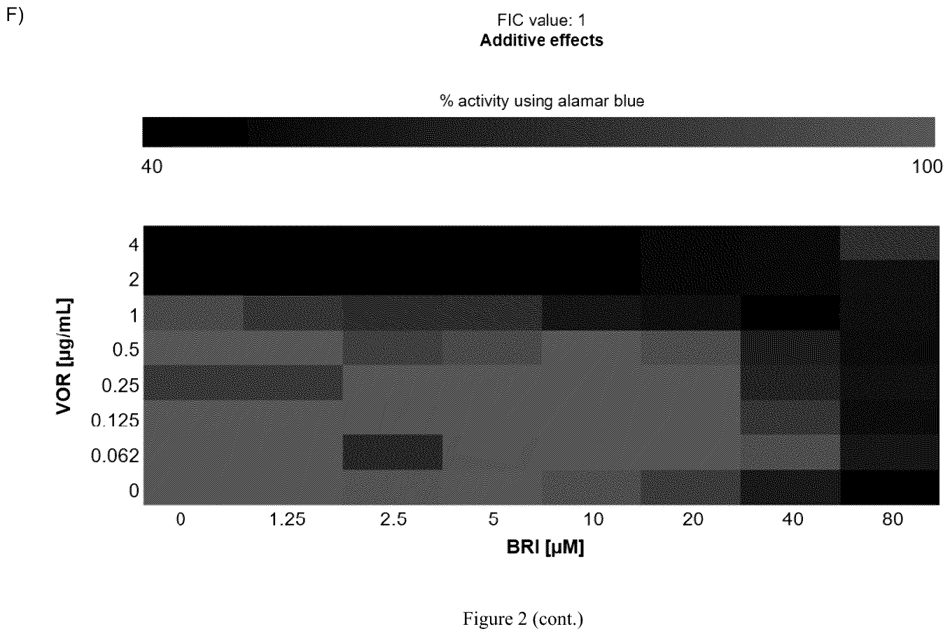

Further tested was performed with BRI. BRI MIC for wild-type A. fumigatus was measured as higher than 80 μM (Table 2) and the A. fumigatus conidial viability was tested after 48 hours of exposure to a combination of CAS 0.2 or 0.5 μg/ml combined with BRI 20 μM (see, FIG. 2, Panel A). The combination of 0.2 or 0.5 μg/ml of CAS with BRI 20 M reduced A. fumigatus conidial viability by 85% and 100%, respectively (see, FIG. 2, Panel A). BRI 20 μM could also synergize with subinhibitory VOR concentrations of 0.125 and 0.25 μg/ml by reducing the A. fumigatus conidial viability by 92% and 99%, respectively (see, FIG. 2, Panel A). See also, FIG. 2, Panel D, for microscopic images of A. fumigatus after 48 hours exposure to BRI 20 μM, CAS 0.5 μg/ml, BRI 20 μM+CAS 0.5 μg/ml, VOR 0.25 μg/ml, and BRI 20 μM+VOR 0.25 μg/ml. The Fractional Inhibitory Concentration (FIC) index for BRI+CAS was 0.39 indicating a synergistic effect while for BRI+VOR FIC index was 1.00 indicating an additive effect against A. fumigatus (see, FIG. 2, Panel E and Panel F).

TABLE 2

| | MEC | MIC | | | | | |
|---|---|---|---|---|---|---|---|
| | CAS | BRI | CAS (μg/mL) | | | BRI (μM) | |
| Strain | (μg/mL) | (μM) | 0.25 | 2 | 4 | 20 | 40 |
| WT | 0.25 | >80 | + | + | + | + | + |
| CM7555 CAS^{R} | 16 | >80 | + | + | + | + | + |
| DPL1033 CAS^{R} | 16 | >80 | + | + | + | + | + |
| MD24053 CAS^{R} | 16 | >80 | + | + | + | + | + |
| CYP-15-184 | 0.25 | >80 | + | ND | ND | + | ND |
| CYP-15-190 | 0.25 | >80 | + | ND | ND | + | ND |
| CYP-15-192 | 0.25 | >80 | + | ND | ND | + | ND |
| CYP-15-195 | 0.25 | >80 | + | ND | ND | + | ND |
| CYP-15-202 | 0.25 | >80 | + | ND | ND | + | ND |
| CYP-15-212 | 0.25 | >80 | + | ND | ND | + | ND |
| CYP-15-213 | 0.25 | >80 | + | ND | ND | − | ND |
| CYP-15-215 | 0.25 | >80 | + | ND | ND | − | ND |
| CYP-15-220 | 0.25 | >80 | + | ND | ND | + | ND |
| CYP-15-221 | 0.25 | >80 | + | ND | ND | + | ND |
| CYP-15-222 | 0.25 | >80 | + | ND | ND | + | ND |
| CYP-15-224 | 0.25 | >80 | + | ND | ND | + | ND |
| CYP-15-225 | 0.25 | >80 | + | ND | ND | + | ND |
| CYP-15-226 | 0.25 | >80 | + | ND | ND | + | ND |
| CYP-15-228 | 0.25 | >80 | + | ND | ND | + | ND |
| CYP-15-229 | 0.25 | >80 | + | ND | ND | + | ND |
| CYP-15-230 | 0.25 | >80 | + | ND | ND | + | ND |
| CYP-15-231 | 0.25 | >80 | + | ND | ND | + | ND |
| CYP-15-75 | 0.25 | >80 | + | ND | ND | + | ND |
| CYP-15-108 | 0.25 | >80 | + | ND | ND | + | ND |
| CYP-15-109 | 0.25 | >80 | + | ND | ND | + | ND |
| CYP-15-147 | 0.25 | >80 | + | ND | ND | + | ND |
| F14946 | 0.25 | >80 | + | ND | ND | + | ND |
| 20089320 | 0.25 | >80 | + | ND | ND | + | ND |

| | 0.25 μg/mL CAS | | 2 μg/mL CAS | | 4 μg/mL CAS | |
|---|---|---|---|---|---|---|
| | 20 μM | 40 μM | 20 μM | 40 μM | 20 μM | 40 μM |
| Strain | BRI | BRI | BRI | BRI | BRI | BRI |
| WT | −−− | −−− | −−− | −−− | −−− | −−− |
| CM7555 CAS^{R} | + | −−− | + | −−− | + | −−− |
| DPL1033 CAS^{R} | + | − | + | − | + | − |
| MD24053 CAS^{R} | + | −−− | + | −−− | + | −−− |
| CYP-15-184 | −−− | ND | ND | ND | ND | ND |
| CYP-15-190 | −−− | ND | ND | ND | ND | ND |
| CYP-15-192 | −−− | ND | ND | ND | ND | ND |
| CYP-15-195 | −−− | ND | ND | ND | ND | ND |
| CYP-15-202 | −−− | ND | ND | ND | ND | ND |
| CYP-15-212 | −−− | ND | ND | ND | ND | ND |
| CYP-15-213 | −−− | ND | ND | ND | ND | ND |
| CYP-15-215 | −−− | ND | ND | ND | ND | ND |
| CYP-15-220 | −−− | ND | ND | ND | ND | ND |
| CYP-15-221 | −−− | ND | ND | ND | ND | ND |
| CYP-15-222 | −−− | ND | ND | ND | ND | ND |
| CYP-15-224 | −−− | ND | ND | ND | ND | ND |
| CYP-15-225 | −−− | ND | ND | ND | ND | ND |
| CYP-15-226 | −−− | ND | ND | ND | ND | ND |
| CYP-15-228 | −−− | ND | ND | ND | ND | ND |
| CYP-15-229 | −−− | ND | ND | ND | ND | ND |
| CYP-15-230 | −−− | ND | ND | ND | ND | ND |
| CYP-15-231 | −−− | ND | ND | ND | ND | ND |
| CYP-15-75 | −−− | ND | ND | ND | ND | ND |
| CYP-15-108 | −−− | ND | ND | ND | ND | ND |
| CYP-15-109 | −−− | ND | ND | ND | ND | ND |
| CYP-15-147 | −−− | ND | ND | ND | ND | ND |
| F14946 | −−− | ND | ND | ND | ND | ND |
| 20089320 | −−− | ND | ND | ND | ND | ND |

Obs.: CAS-caspofungin; BRI = Brilacidin.
CM7555, DPL1033, and MD24053 are clinical isolates resistant to caspofungin. The mechanism of CASR in CM7555 is unknown while strains DPL1033 and MD24053 have mutations S679P and F675S in the fks1 gene.
(+): little growth; (−): partial inhibition; and (−−−) total inhibition; ND = not determined

Antimicrobial peptides target directly or indirectly the microorganism plasma membrane disrupting their membrane potential (Lima et al., Life Sci., 2021, 278, 119647; and Veerana et al., Microbial. Biotechnol., 2021, 14, 262-276), and BRI acts by a similar mechanism (Mensa et al., Antimicrob. Agents Chemother., 2014, 58, 5136-45; Tew et al., Proc. Natl. Acad. Sci. USA, 2002, 99, 5110-5114; Tew et al., Acc. Chem. Res., 2010, 43, 30-9). The effect of BRI+CAS on the resting membrane potential was determined by using the fluorescent voltage reporter DIBAC+(3) (increase in the fluorescent intensity indicates membrane depolarization; see, FIG. 2, Panel B). Untreated A. fumigatus had 0% fluorescent germlings while exposure to either CAS 0.125 μg/ml, BRI I μM, or a combination of both resulted in ˜25%, 5%, and 80% fluorescent germlings, respectively (see, FIG. 2, Panel B). Germlings previously transferred to MM without glucose (non-carbon source, NCS) for 4 hours before adding CAS, BRI, or CAS+BRI for 30 minutes and subsequently DIBAC+(3) have not shown any fluorescent difference with the cells that were grown in the presence of the carbon source glucose (see, FIG. 2, Panel B). These results suggest that BRI as well as CAS is passively transported through the cell membrane without the need for ATP. A. fumigatus biofilm formation is more resistant to antifungal agents (Morelli et al., PLOS Pathog., 2021, 17, e1009794). BRI alone at 20 or 40 μM can inhibit about 10% and 60% biofilm formation, respectively (see, FIG. 2, Panel C) while VOR alone at 2 μg/ml or 4 μg/ml were able to inhibit about 10% and 50% biofilm formation (see, FIG. 2, Panel C); CAS alone at 0.25 μg/ml or 2.0 μg/ml were able to inhibit about 10% biofilm formation (see, FIG. 2, Panel C). The different combinations BRI+VOR or BRI+CAS were able to potentiate the inhibition of the biofilm formation about 50 to 90% (see, FIG. 2, Panel C).

The combination of BRI+CAS was evaluated to determine whether it is able to inhibit CAS-resistant and VOR-resistant A. fumigatus clinical isolates (see, Table 2). CAS 0.25 to 4 μg/ml with BRI at 20 and 40 μM and VOR at concentrations of 0.5 and 2 μg/ml with BRI at 20 and 40 μM were tested. BRI had no activity against 25 A. fumigatus clinical isolates susceptible to CAS (MEC CAS of 0.25 μg/ml) and 3 CAS-resistant clinical strains (MEC CAS of 16 μg/ml; strains DPL1033, and MD24053 with known fks1 mutations; and strain CM7555 with an unknown mutation(s)). Interestingly, addition of BRI at 20 or 40 μM to CAS either partially or completely inhibited the growth of all tested strains including those that are resistant to CAS or with known resistance to azoles (Table 2). Thus, BRI clearly potentiates CAS activity against CAS- or VOR-resistant strains of A. fumigatus.

In contrast to the potentiation of CAS activity by BRI, addition of BRI (at 20 or 40 μM) to VOR had no effect on the resistant nature of 22 clinical isolates (15 strains with the TR^{34/}L98H mutation and 7 strains with unknown mutation(s)) to VOR (Table 3). In the experiment, effects were observed for 2 clinical isolates: one strain (CYP15-15-109) was totally inhibited by VOR, showing wild-type response; and one other strain (CYP15-15-147) was partially inhibited by VOR 0.5 μg/ml+BRI but the inhibition was not seen at VOR 2.0 μg/ml+BRI (Table 3). Curiously, the VOR-resistant clinical isolates were not inhibited by a combination of BRI+VOR but they were inhibited by BRI+CAS (compare Table 2 with Table 3). Most of the VOR-resistant strains have increased accumulation of ergosterol since the tandem-repeat mutations at the promoter region increase the erg11A expression and consequently the ergosterol production (Hagiwara et al., Front. Microbiol., 2016, 7, 1382). Ergosterol is essential for the integrity and fluidity of fungal cell membranes and azole-induced depletion of ergosterol alters the membrane sterol composition, its stability and arrests fungal growth (Shapiro et al., Microbiol. Mol. Biol. Rev., 2011, 75, 213-267).

TABLE 3

| | | MIC | MIC | | |
|---|---|---|---|---|---|
| | | VOR | BRI | VOR (μg/ml) | |
| Strain | VOR^{R} | (μg/ml) | (μM) | 0.5 | 2 |
| WT (CEA17) | WT | 2 | 80 | −−− | −−− |
| CYP-15-184 | TR34/L98H | 8 | 80 | + | + |
| CYP-15-190 | TR34/L98H | 8 | 80 | + | + |
| CYP-15-192 | TR34/L98H | 4 | 80 | + | + |
| CYP-15-195 | TR34/L98H | 8 | 80 | + | + |
| CYP-15-202 | TR34/L98H | 8 | 80 | + | + |
| CYP-15-212 | TR34/L98H | 8 | 80 | + | + |
| CYP-15-213 | TR34/L98H | 4 | 80 | + | + |
| CYP-15-215 | Unknown | 8 | 80 | + | + |
| CYP-15-220 | TR34/L98H | 4 | 80 | + | + |
| CYP-15-221 | TR34/L98H | 8 | 80 | + | + |
| CYP-15-222 | TR34/L98H | 4 | 80 | + | + |
| CYP-15-224 | Unknown | 8 | 80 | + | + |
| CYP-15-225 | Unknown | 8 | 80 | + | + |
| CYP-15-226 | TR34/L98H | 4 | 80 | + | + |
| CYP-15-228 | TR34/L98H | 4 | 80 | + | + |
| CYP-15-229 | TR34/L98H | 8 | 80 | + | + |
| CYP-15-230 | TR34/L98H | 8 | 80 | + | + |
| CYP-15-231 | TR34/L98H | 4 | 80 | + | + |
| CYP-15-75 | Unknown | 8 | 80 | + | + |
| CYP-15-108 | Unknown | 8 | 80 | + | + |
| CYP-15-109 | Unknown | >8 | 80 | −−− | −−− |
| CYP-15-147 | Unknown | 8 | 80 | + | + |
| F14946 | Unknown | 8 | 80 | + | + |
| 20089320 | Unknown | 8 | 80 | + | + |

| | | BRI | 0.5 μg/ml VOR | | 2 μg/ml VOR | |
|---|---|---|---|---|---|---|
| | | (μM) | 20 μM | 40 μM | 20 μM | 40 μM |
| Strain | VOR^{R} | 20 | BRI | BRI | BRI | BRI |
| WT (CEA17) | WT | + | −−− | −−− | −−− | −−− |
| CYP-15-184 | TR34/L98H | + | + | + | + | + |
| CYP-15-190 | TR34/L98H | + | + | + | + | + |
| CYP-15-192 | TR34/L98H | + | + | + | + | + |
| CYP-15-195 | TR34/L98H | + | + | + | + | + |
| CYP-15-202 | TR34/L98H | + | + | + | + | + |
| CYP-15-212 | TR34/L98H | + | + | + | + | + |
| CYP-15-213 | TR34/L98H | − | + | + | + | + |
| CYP-15-215 | Unknown | − | + | + | + | + |
| CYP-15-220 | TR34/L98H | + | + | + | + | + |
| CYP-15-221 | TR34/L98H | + | + | + | + | + |
| CYP-15-222 | TR34/L98H | + | + | + | + | + |
| CYP-15-224 | Unknown | + | + | + | + | + |
| CYP-15-225 | Unknown | + | + | + | + | + |
| CYP-15-226 | TR34/L98H | + | + | + | + | + |
| CYP-15-228 | TR34/L98H | + | + | + | + | + |
| CYP-15-229 | TR34/L98H | + | + | + | + | + |
| CYP-15-230 | TR34/L98H | + | + | + | + | + |
| CYP-15-231 | TR34/L98H | + | + | + | + | + |
| CYP-15-75 | Unknown | + | + | + | + | + |
| CYP-15-108 | Unknown | + | + | + | + | + |
| CYP-15-109 | Unknown | + | −−− | −−− | −−− | −−− |
| CYP-15-147 | Unknown | + | − | − | + | + |
| F14946 | Unknown | + | + | + | + | + |
| 20089320 | Unknown | + | + | + | + | + |

Obs.: 1) VOR-voriconazole; BRI = Brilacidin.
(+): no inhibition; (−): partial inhibition; and (−−−) total inhibition 2) In the experiment, one strain (CYP15-15-109) was totally inhibited by VOR, showing Wild Type response; also, one strain (CYP15-15-147) was partially inhibited by VOR 0.5 μg/ml + BRI but the inhibition was not seen at VOR 2.0 μg/ml + BRI.

Taken together, these results indicate that the combination CAS+BRI can depolarize the cell membrane converting CAS from a fungistatic into a fungicidal drug for A. fumigatus. BRI+CAS can decrease A. fumigatus biofilm formation and completely or partially overcome CAS-resistance in echinocandin-resistant A. fumigatus clinical isolates. VOR-resistant isolates are sensitive to CAS+BRI combinations but most of them are not sensitive to BRI+VOR combinations.

### Example 3: BRI is Impacting A. fumigatus Calcineurin Signaling and the Cell Wall Integrity (CWI) Pathway

To assess the mechanism of action of BRI, a collection of 58 protein kinase inhibitors (PKI, at a concentration of 20 μM; Table 4) was screened for A. fumigatus growth and corresponding metabolic activity alone or together with 20 μM BRI (Table 4).

TABLE 4

| COMPOUND | TARGET | PKI (20 | PKI (20 μM) + | molecular weight |
|---|---|---|---|---|
| NAME | KINASE | μM) | BRI (20 μM) | (MW) |
| PF + 3758309 | PAK3 | ++ | ++ | 490.634 |
| FRAX597 | | ++ | ++ | 558.108 |
| FRAX486 | | ++ | + | 513.403 |
| G5555 | | ++ | ++ | 492.966 |
| GW300660X | PRKAA1 | ++ | ++ | 453.482 |
| GW296115X | | ++ | ++ | 385.378 |
| GW416981X | | ++ | ++ | 415.469 |
| GW290597X | | ++ | ++ | 467.509 |
| CHIR + 99021 | GSK3A | ++ | ++ | 465.347 |
| SB 216763 | | ++ | ++ | 371.222 |
| LY2090314 | | ++ | ++ | 512.543 |
| AZD1080 | | ++ | ++ | 334.378 |
| AZD + 8055 | MTOR | ++ | ++ | 465.552 |
| vistusertib | | ++ | ++ | 462.552 |
| Torin 2 | | ++ | ++ | 432.404 |
| CC + 115 | | ++ | ++ | 336.358 |
| D4476 | CSNK1G2 | ++ | ++ | 398.421 |
| NK + 258 | | ++ | ++ | 322.289 |
| PP121 | STK25 | ++ | + | 319.371 |
| BX + 795 | | ++ | ++ | 591.472 |
| SCH772984 | MAPK3 | ++ | ++ | 587.686 |
| | (ERK1) | | | |
| GW305074X | | ++ | ++ | 520.942 |
| XMD8 + 92 | | ++ | ++ | 474.563 |
| GW300657X | | ++ | ++ | 450.478 |
| PF + 4708671 | RPS6KB2 | ++ | ++ | 390.412 |
| LY + 2584702 | | ++ | ++ | 617.626 |
| AT7867 | | ++ | ++ | 410.775 |
| VX + 745 | MAPK11 | ++ | ++ | 436.268 |
| AKI00000001a | | ++ | ++ | 396.4 |
| AKI00000067a | | ++ | ++ | 379.369 |
| GSK223810A | | ++ | ++ | 513.596 |
| ralimetinib | | ++ | ++ | 612.746 |
| TAK + 715 | CSNK1E | ++ | ++ | 399.517 |
| PF + 670462 | | ++ | ++ | 410.323 |
| CK1 + IN + 1 | | ++ | ++ | 383.4 |
| PF + 5006739 | | ++ | ++ | 419.463 |
| MK + 8353 | MAPK1 | ++ | ++ | 691.859 |
| PHA + 767491 | CDC7 | ++ | ++ | 213.239 |
| XL413 | | ++ | ++ | 289.721 |
| LY3177833 | | ++ | ++ | 309.303 |
| refametinib | MAP2K2 | ++ | ++ | 572.337 |
| selumetinib | | ++ | ++ | 457.686 |
| SR + 3677 | CAMK1G | ++ | ++ | 481.378 |
| CX + 4945 | CSNK2A2 | ++ | ++ | 349.776 |
| SGC + CK2 + 1 | CSNK2A2 | ++ | ++ | 375.435 |
| TTP 22 | | ++ | ++ | 330.4 |
| Sorafenib | MAPK13 | ++ | ++ | 464.83 |
| 5 + iodo | MAP2K3 | + | ++ | 392.148 |
| tubercidin | | | | |
| SB + 216385 | | ++ | ++ | 382.442 |
| SB + 203580 | PRKCA | ++ | ++ | 377.442 |
| enzastaurin | | ++ | ++ | 515.615 |
| darovasertib | | ++ | ++ | 472.474 |
| GW461487A | | ++ | ++ | 339.8 |
| UNC + ALM + 33 | | ++ | ++ | 303.339 |
| UNC + ALM + 39 | | + | ++ | 304.327 |
| UNC + ALM + 16 | | + | ++ | 304.327 |
| UNC + ALM + 87 | | ++ | ++ | 309.367 |
| GW434756X | | ++ | ++ | 289.31 |

++: growth ≥80% compared to no treatment
+: growth ≤60% compared to no treatment

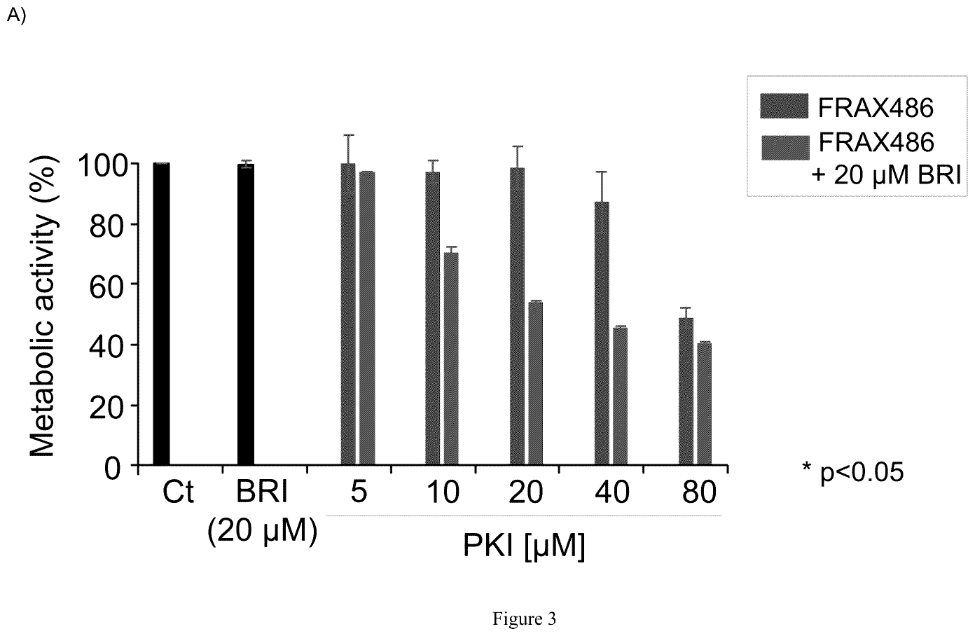

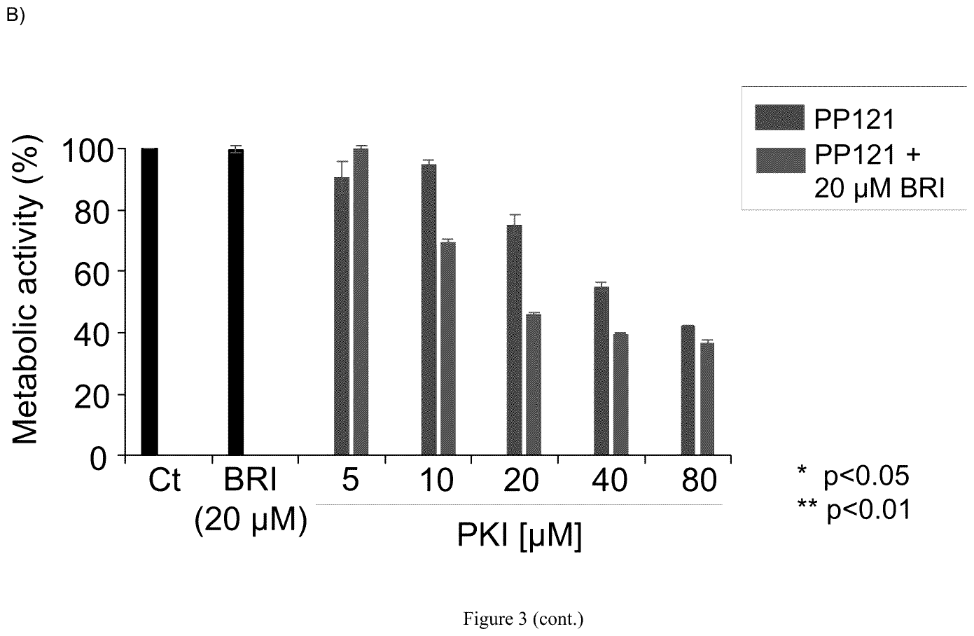

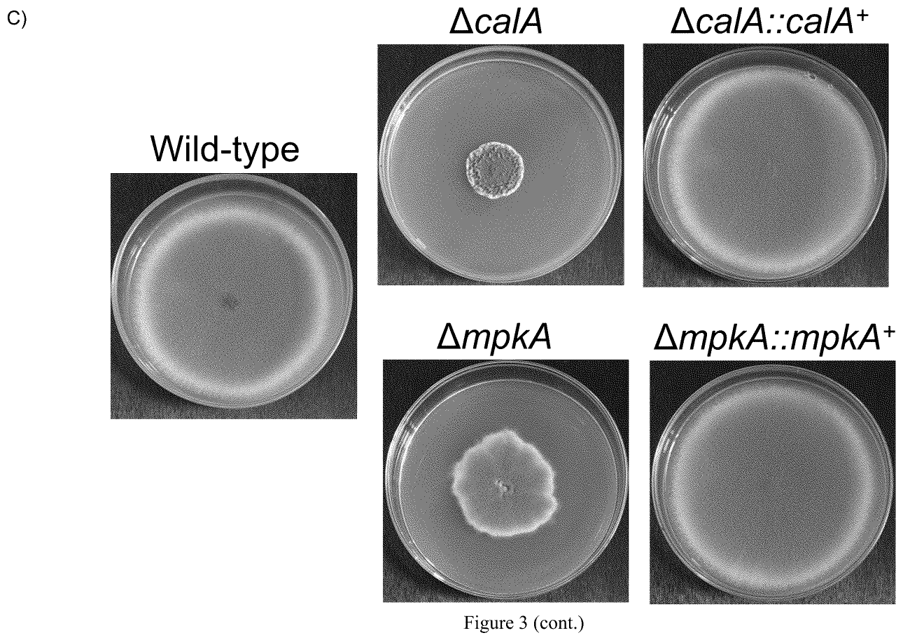

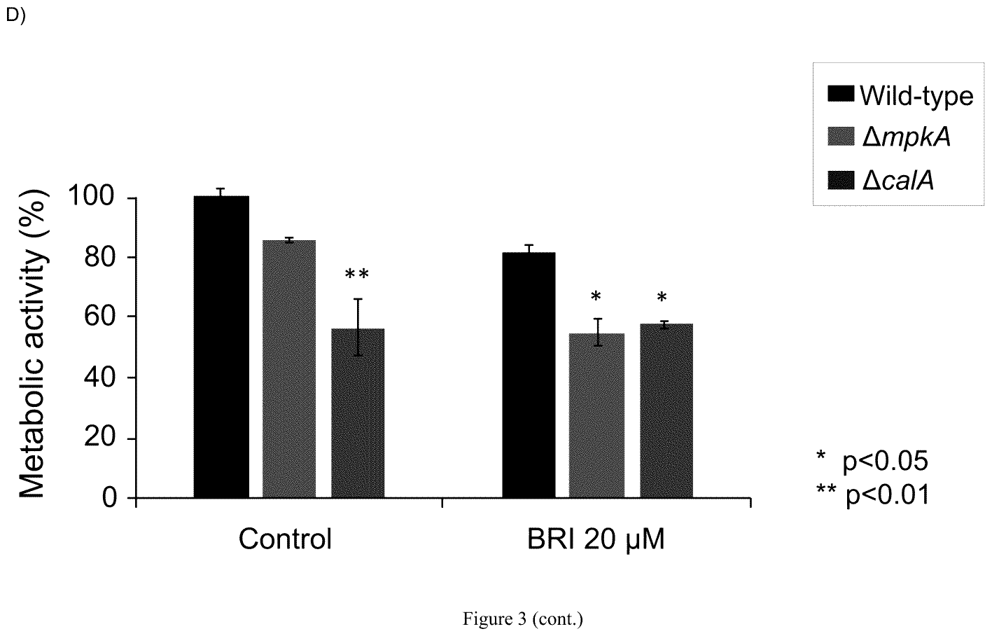

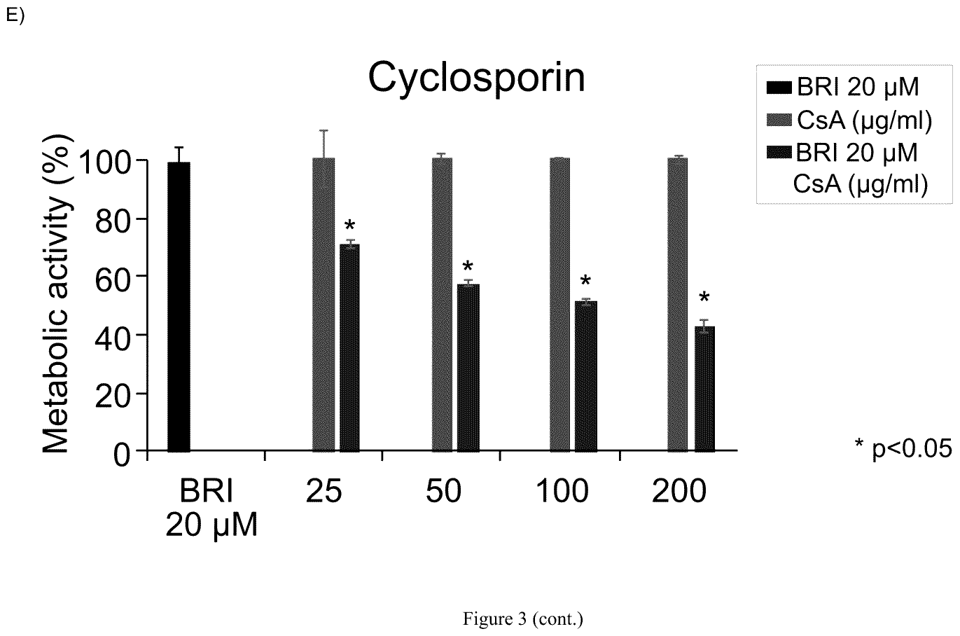

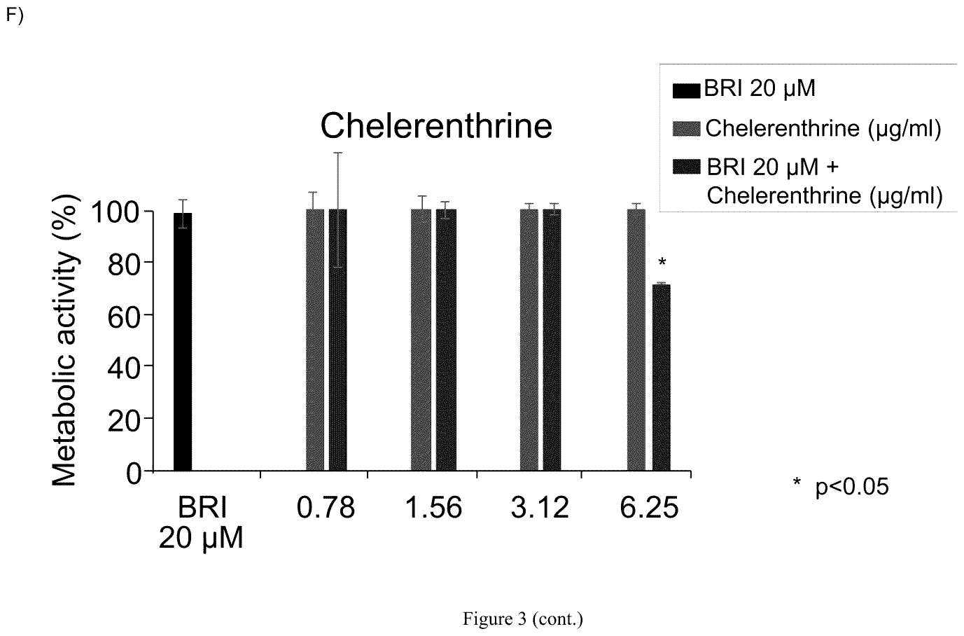

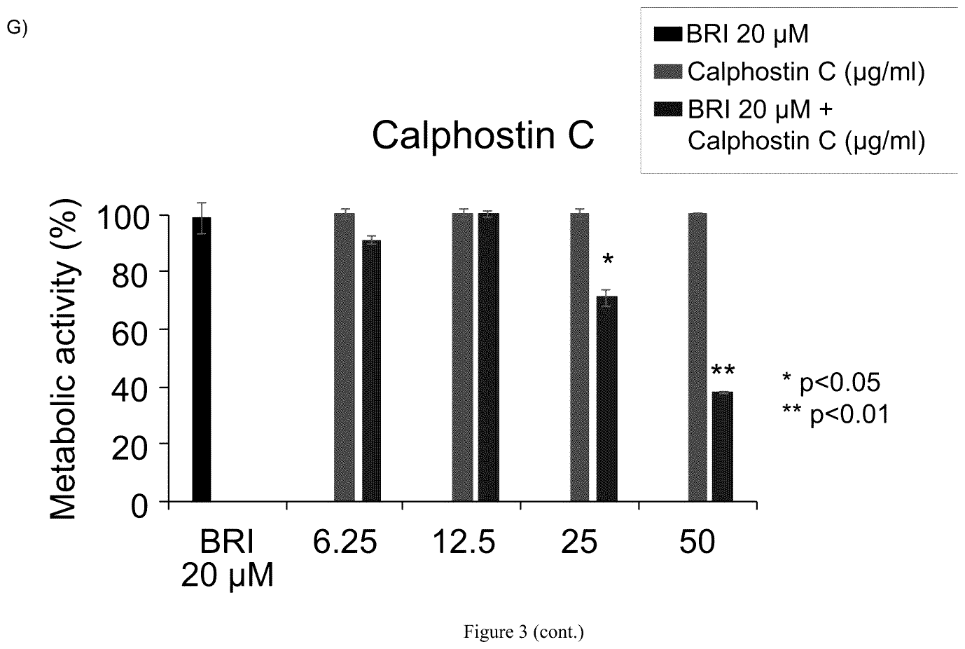

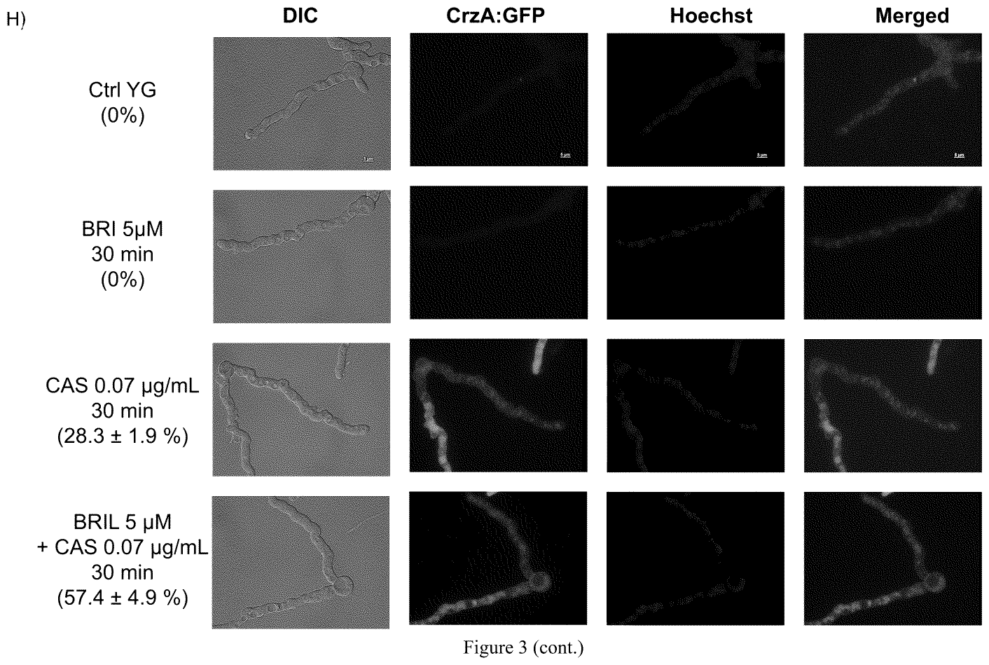

Two PKIs, a p21-Activated Kinase Inhibitor FRAX486 and a STK25 inhibitor PP121, both members of the sterile 20 superfamily of kinases, are identified as potentiating the BRI activity against A. fumigatus (see, FIG. 3, Panels A and B). The p21 activated kinases (PAKs) belongs to the family of Ste20-related kinases and these kinases have been shown to be involved in signaling through mitogen activated protein kinase (MAPK) pathways (Boyce et al., Trends Microbiol., 2011, 19, 400-10). The closest STK25 homologues are cAMP-mediated signaling proteins Sok1p in Saccharomyces cerevisiae whose overexpression suppresses the growth defect of mutants lacking protein kinase A activity (Ward et al., Mol. Cell. Biol., 1994, 14, 5619-27). A library of 25 A. fumigatus null mutants for phosphatase catalytic subunits (Winkelströter et al., G3 (Bethesda), 2015, 5, 1525-39) was also screened for sensitivity to BRI 20 μM. A single phosphatase mutant, ΔcalA (calA encodes the calcineurin catalytic subunit), was identified as more sensitive to BRI 20 μM (Table 5).

TABLE 5

| | | |
|---|---|---|
| Strain | CAS (0.25 μg/mL) | BRI (20 μM) |

reference strains

| | | |
|---|---|---|
| CEA17 (wild-type; A1160) | + | + |
| Afs35 (wild-type; A1159) | + | + |

phosphatase null mutants

| | | |
|---|---|---|
| ΔpphA (Afu5g12010) | + | + |
| ΔsitA (Afu6g11470) | + | + |
| ΔppzA (Afu2g03950) | + | + |
| ΔpptA (Afu5g06700) | + | + |
| ΔptcA (Afu1g15800) | + | + |
| ΔptcB (Afu1g09280) | + | + |
| ΔppmA (Afu8g04580) | + | + |
| ΔptcD (Afu5g13740) | + | + |
| ΔptcE (Afu2g03890) | + | + |
| ΔptcF (Afu1g06860) | + | + |
| ΔptcG (Afu5g13340) | + | + |
| ΔptcH (Afu4g00720) | + | + |
| ΔnemA (Afu1g09460) | + | + |
| ΔpsrA (Afu1g04790) | + | + |
| ΔppsA (Afu5g11690) | + | + |
| ΔdspD (Afu2g02760) | + | + |
| ΔcdcA (Afu3g12250) | + | + |
| ΔdspA (Afu1g13040) | + | + |
| ΔdspB (Afu1g03540) | + | + |
| ΔptpB (Afu3g10970) | + | + |
| ΔpypA (Afu4g04710) | + | + |
| ΔltpA (Afu2g01880) | + | + |
| ΔyphA (Afu4g07000) | + | + |
| ΔptyA (Afu6g06650) | + | + |
| ΔcalA (Afu5g09360) | + | − |

kinases null mutants

| | | |
|---|---|---|
| ΔmpkA | − | − |
| ΔmpkB | + | + |
| ΔmpkC | + | + |
| ΔsakA | + | + |
| ΔsakA; ΔmpkC | + | + |

+: sensitivity similar to wild-type
−: more sensitive than the wild-type

Considering the importance of calcineurin in the signaling response to A. fumigatus osmotic stress and the cell wall integrity pathway (da Silva Ferreira et al., Fungal Genet, Biol., 2007, 44, 219-30; Ries et al., mBio., 2017, 8, e00705-17; de Castro et al., Mol. Microbiol., 2014, 94, 655-74; de Castro et al., PLOS Genet., 2019, 15, e1008551) and the fact that PAKs have been shown to be involved in signaling through MAPK pathways (Boyce et al., Trends Microbiol., 2011, 19, 400-10), four A. fumigatus null mutants for MAPK ΔsakA, ΔmpkC, ΔmpkB and ΔmpkA were tested for BRI growth inhibition. Only ΔmpkA was identified as more sensitive to BRI 20 μM (Table 5). Both mutants ΔcalA and ΔmpkA have severe growth defects (see, FIG. 3, Panel C) and further validation by using Alamar blue metabolic activity showed that ΔcalA strain had similar metabolic activity in the presence or absence of BRI (see, FIG. 3, Panel D). In contrast, ΔmpkA mutant had a significantly decreased metabolic activity when grown in the presence of BRI (see, FIG. 3, Panel D). Cyclosporine (CsA) is a specific inhibitor of calcineurin and there is synergy, in inhibition of A. fumigatus metabolic activity, between increasing concentrations of CsA and BRI 20 μM (see, FIG. 3, Panel E). A. fumigatus protein kinase C (PKC) is important for the activation of the CWI pathway (Rocha et al., PLOS One, 2015, 10, e0135195) and PKC inhibitors such as chelerenthrine and calphostin C also synergize with BRI 20 μM (see, FIG. 3, Panels F and G). A. fumigatus calcineurin regulates the activity of the Crz.A transcription factor with the phosphorylated form accumulating in the cell cytosol, and in response to several stimuli, including CAS exposure, calcineurin dephosphorylates CrzA leading to its re-localization to the nucleus (Ries et al., mBio., 2017, 8, e00705-17; de Castro et al., Mol. Microbiol., 2014, 94, 655-74; de Castro et al., PLOS Genet., 2019, 15, e1008551). When a functional CrzA:GFP strain is not exposed to any drug, or exposed to BRI (5 μM), or to a sub-inhibitory concentration of CAS (0.07 μg/ml), 0, 0) and 28.3%, respectively, of the germlings have

CrzA:GFP in the nuclei, while 57.4% are in the nuclei when this strain is exposed to a combination of BRI+CAS (see, FIG. 3, Panel H).

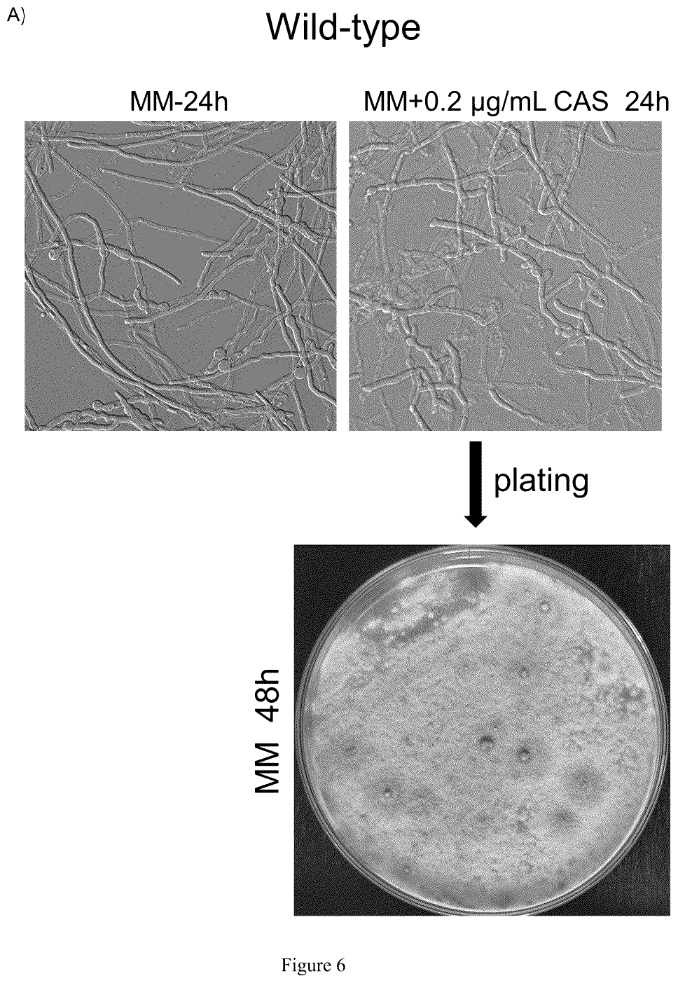

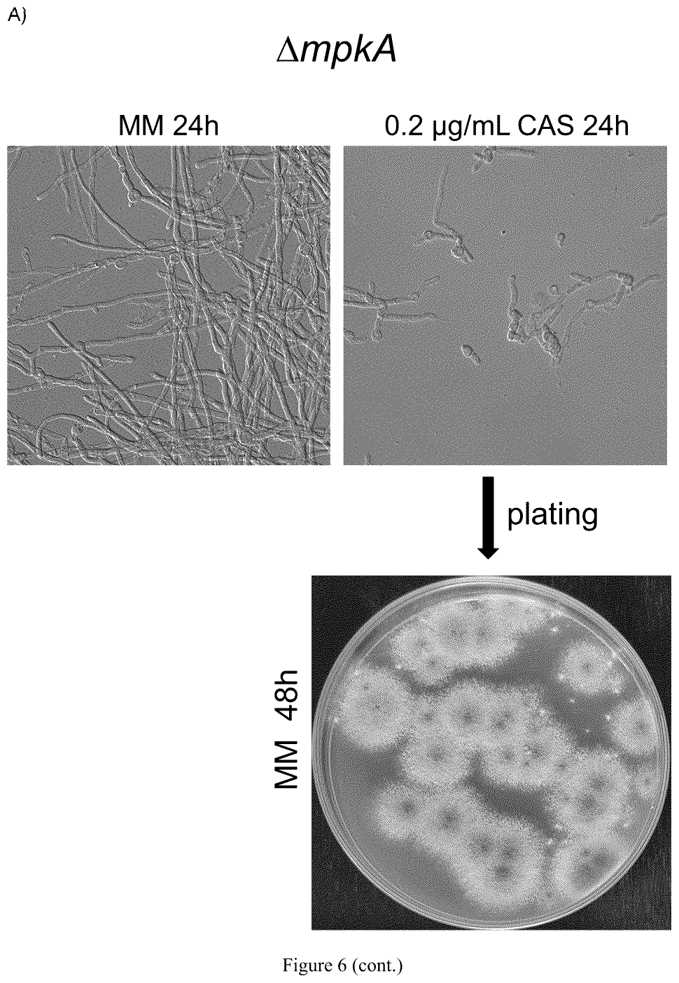

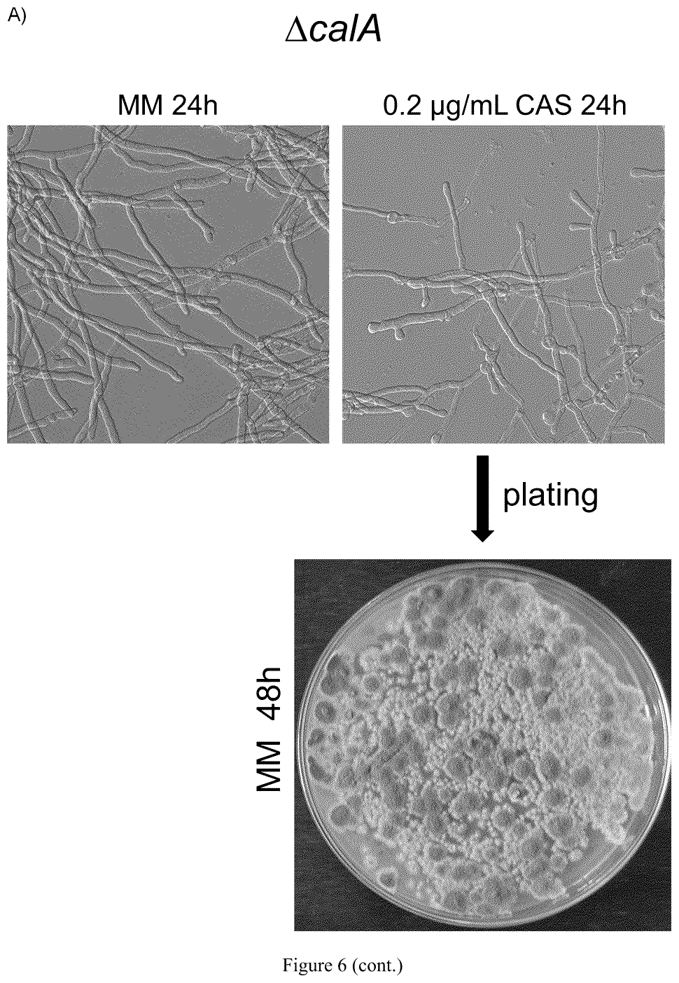

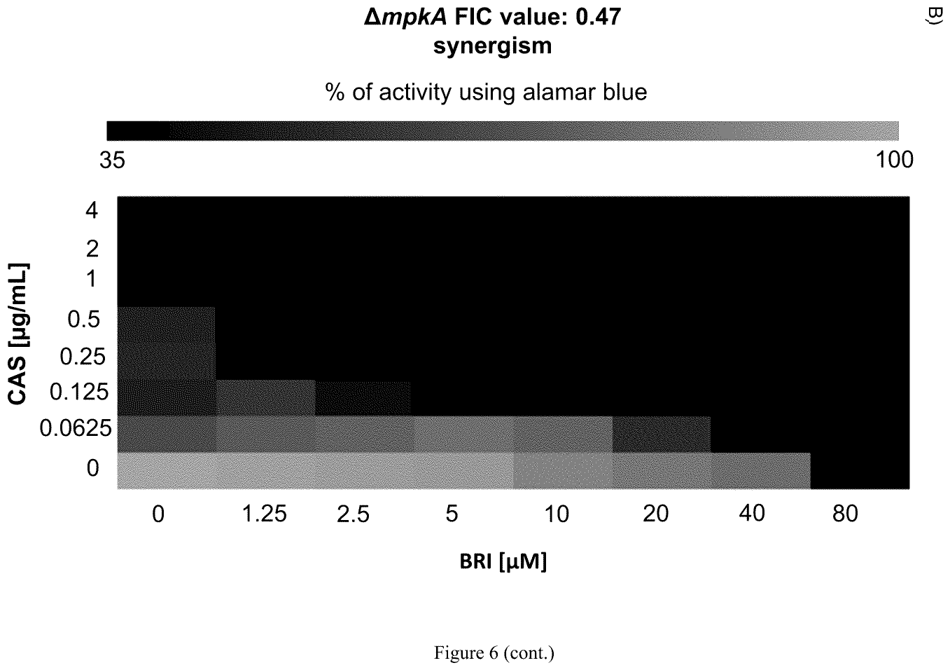

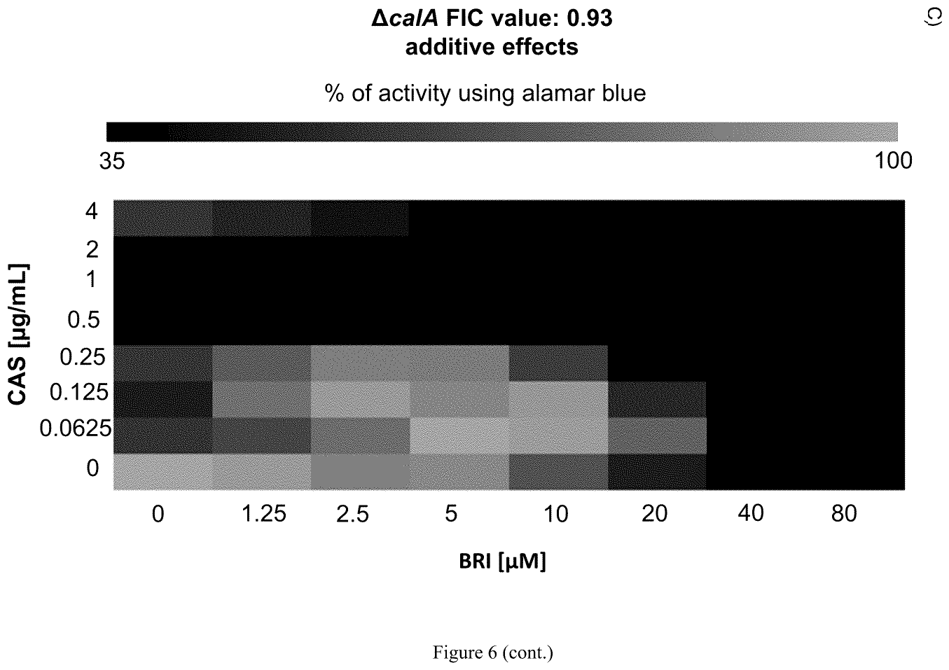

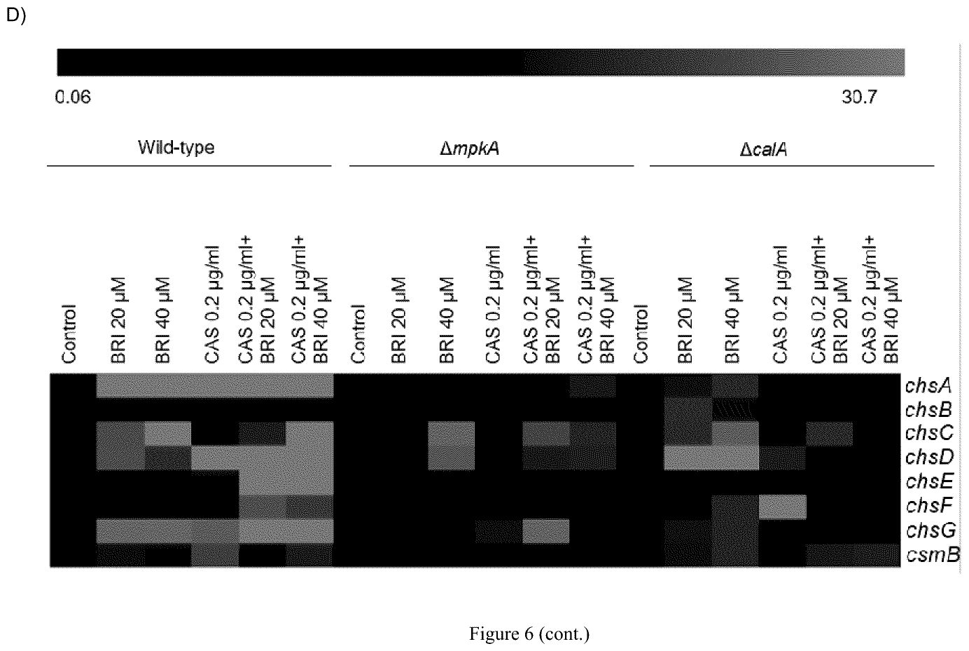

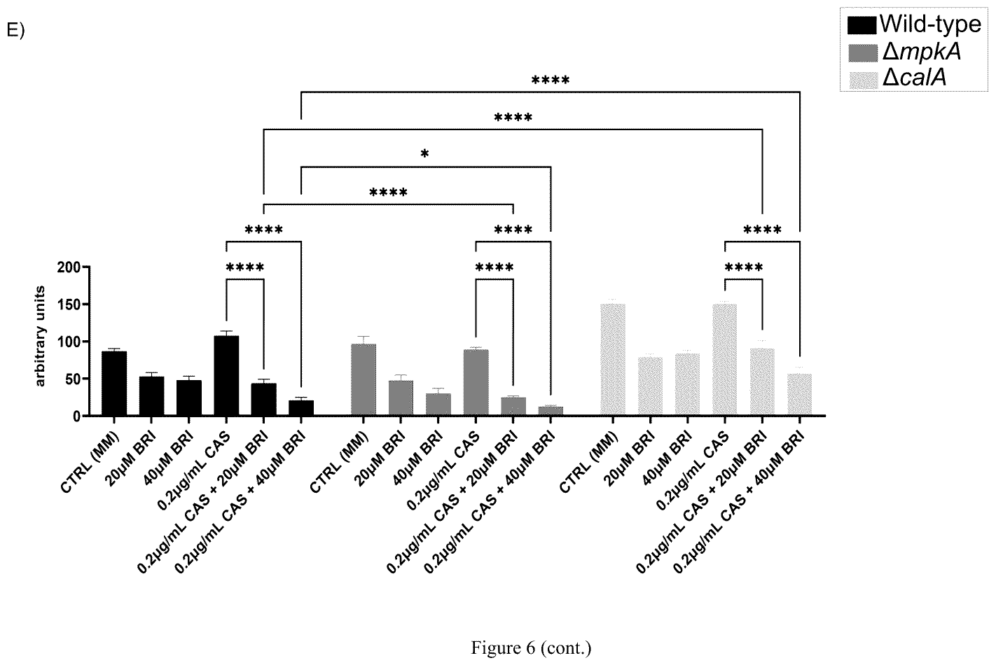

To further validate the involvement of BRI in the CWI pathway mediated by MpkA, and CalA, several phenotypic parameters related to this pathway in the wild-type and mutant strains were comparatively evaluated. Caspofungin shows a fungistatic activity not only against the A. fumigatus wild-type but also against ΔmpkA, and ΔcalA mutant strains (see, FIG. 6, Panel A). The relationship between BRI and CAS was compared in a checkerboard assay with the corresponding wild-type and null mutant strains (contrast FIG. 2, Panel E to FIG. 6, Panel B and Panel C). There is an increased susceptibility of ΔmpkA and ΔcalA to lower combinations of BRI+CAS when compared to the wild-type strain (FIG. 6, Panel B and Panel C). The FIC index for these two drugs in the wild-type and the mutant strains is 0.47 and 0.93, indicating synergistic and additive effects for ΔmpkA and ΔcalA, respectively (FIG. 6, Panel B and Panel C). Chitin overproduction stabilizes the cell wall at early stages of CAS exposure but this increase by itself is not able to confer resistance to the drug (Ries et al., mBio., 2017, 8, e00705-17). A. fumigatus has 8 chitin synthase genes and the increased expression of some of these genes, a hallmark for the response to CAS (Ries et al., mBio., 2017, 8, e00705-17), was also globally lower in the null mutants than in the wild-type strain when they were exposed to CAS or CAS+BRI (see, FIG. 6, Panel D). Interestingly, not only CAS and CAS+BRI but also BRI alone were able to induce some of these genes in the wild-type strain but not in the mutant strains (see, FIG. 6, Panel D). The germling exposure of chitin, as measured by Calcofluor White (CFW, that recognizes chitin) staining, is dysregulated in the mutant strains: lower in the ΔmpkA but higher in ΔcalA mutants than in the wild-type strain (see, FIG. 6, Panel E).

Taken together, these results strongly indicate that CalA and MpkA are important for BRI activity, and BRI is most likely impacting the A. fumigatus CWI pathway.

### Example 4: BRI can Potentiate Caspofungin Activity in C. neoformans, C. albicans, and C. auris

It was investigated whether BRI could potentiate CAS activity in other human fungal pathogens, such as C. neoformans, C. albicans, and C. auris. The MICs for BRI in C. neoformans, C. albicans, and C. auris are 2.5 μM, 80 μM and 80 μM, respectively (see, Table 6).

TABLE 6

| | MIC | | MIC BRI | |
|---|---|---|---|---|
| Species | CAS (μg/ml) | FKS1 mutation | (μM) | Mechanism |
| Cryptococcus | 32.0 | ND | 2.5 | Fungicidal |
| neoformans H99 | | | | |
| Candida albicans | 0.25 | ND | 80 | Fungicidal |
| SC5314 | | | | |
| Candida albicans | 2.0 | F641L | 20 | Fungicidal |
| DPL1006 CAS^{R} | | | | |
| Candida albicans | 4.0 | F641S | 80 | Fungicidal |
| DPL1007 CAS^{R} | | | | |
| Candida albicans | 4.0 | S645Y | 20 | Fungicidal |
| DPL1009 CAS^{R} | | | | |
| Candida albicans | 2.0 | S645F | 80 | Fungicidal |
| DPL1010 CAS^{R} | | | | |
| Candida albicans | 4.0 | S645F + R1361R/H | 40 | Fungicidal |
| DPL1011 CAS^{R} | | | | |
| Candida auris | 1.0 | ND | 80 | Fungicidal |
| 467/2015 | | | | |
| Candida auris | 1.0 | ND | 80 | Fungicidal |
| 468/2015 | | | | |
| Candida auris | 1.0 | ND | 80 | Fungicidal |
| 469/2015 | | | | |
| Candida auris | 1.0 | ND | 80 | Fungicidal |
| 470/2015 | | | | |
| Candida auris | 1.0 | ND | 80 | Fungicidal |
| 471/2015 | | | | |

Obs.: Fungicidal activity was measured by plating the cells or conidia after 48 hours incubation with the different drug concentrations on solid minimal medium and calculating the colony forming units divided by the initial inoculum;
ND = Not determined

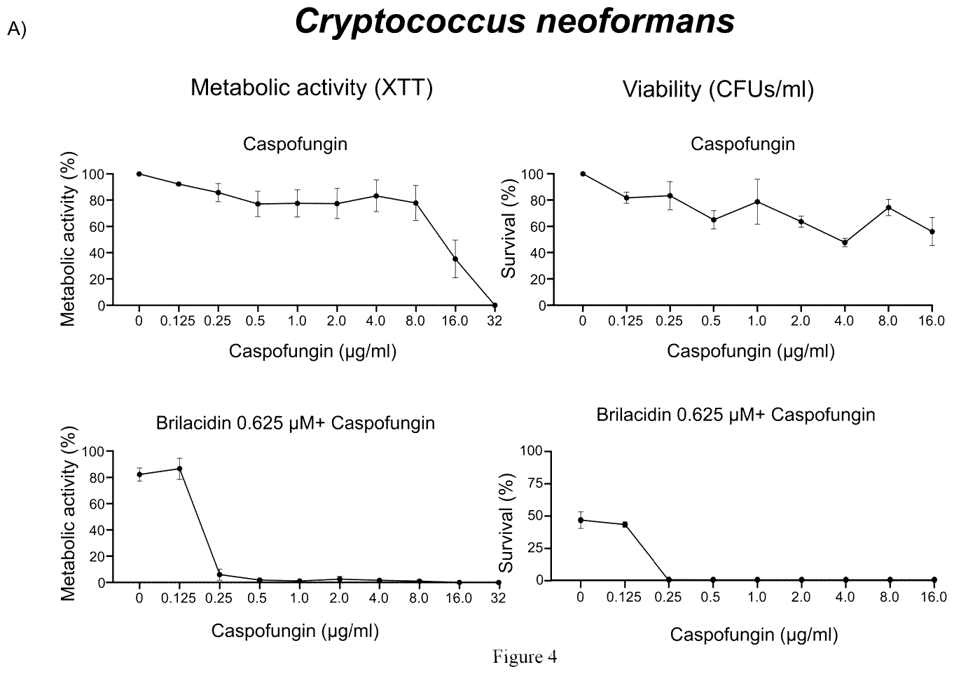

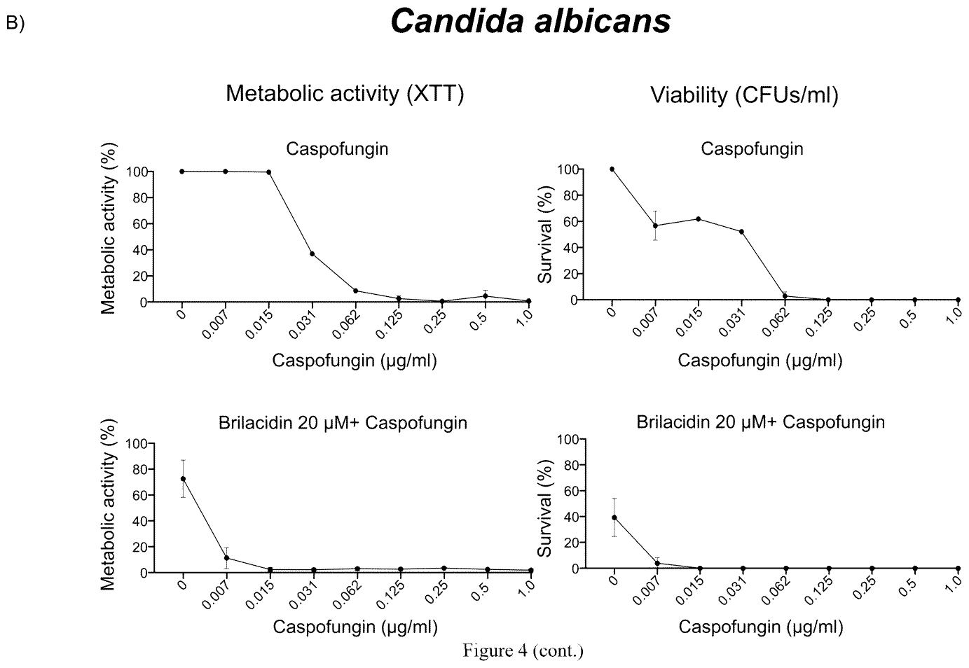

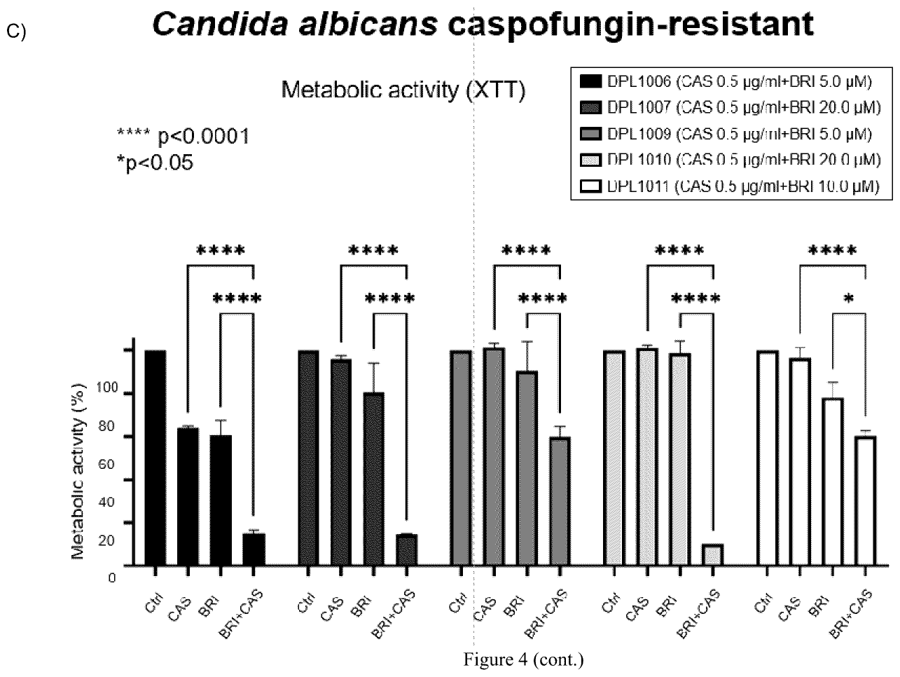

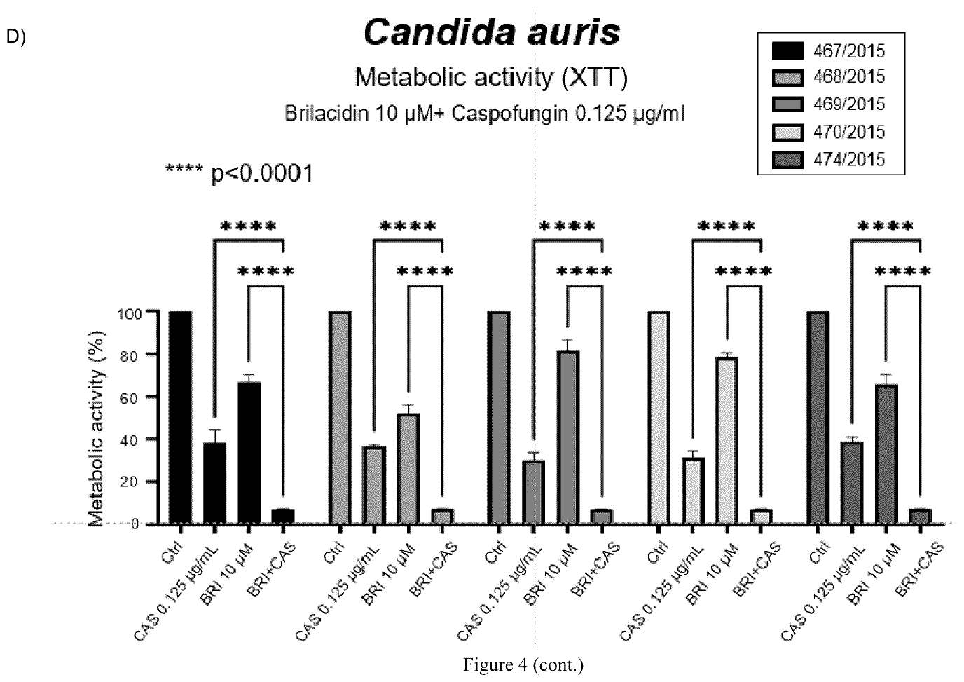

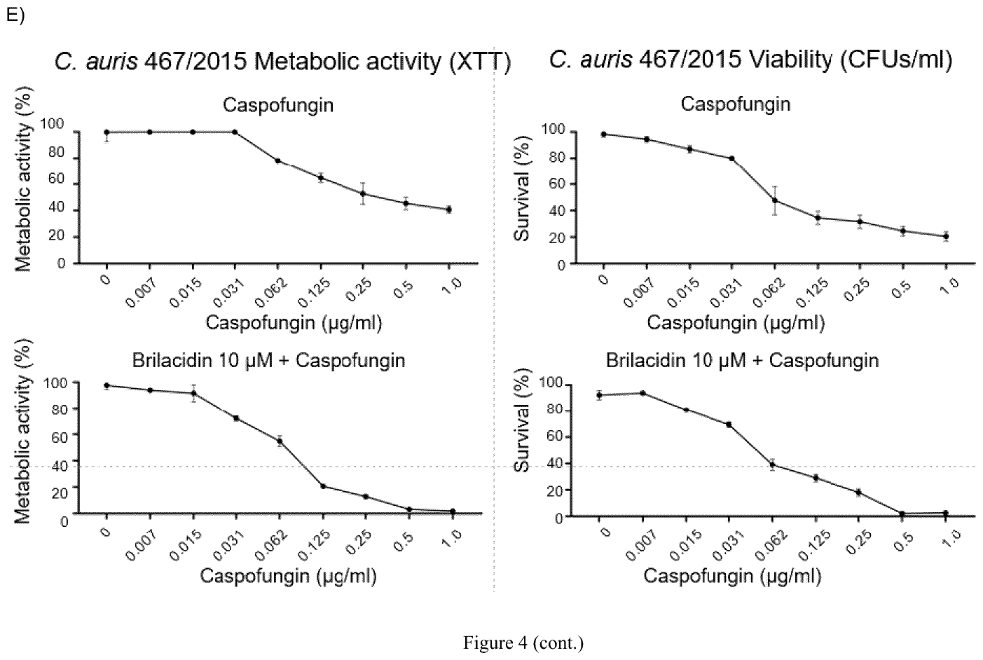

CAS lacks significant activity against C. neoformans (Johnson et al., Expert Opin. Pharmacother., 2003, 4, 807-23) and only high CAS concentrations, such as CAS 32 μg/ml can completely inhibit C. neoformans metabolic activity (as determined by XTT) and CAS 16 μg/ml can decrease survival (colony forming units, CFUs) by about 50% (see, FIG. 4, Panel A). BRI 0.625 μM (0.25× MIC) potentiates CAS activity (0.25 to 0.5 μg/ml of CAS) resulting in complete inhibition of C. neoformans metabolic activity and growth (see, FIG. 4, Panel A). Similarly, complete inhibition of C. albicans metabolic activity (XTT) and survival (CFUs) shifted from a concentration of CAS at ˜0.125 μg/ml in the absence of BRI to a concentration of 0.015 μg/ml of CAS when 20 μM of BRI is added (i.e., an 8-fold reduction in the MIC of CAS) (see, FIG. 4, Panel B). BRI also partially suppressed the CAS-resistance of (. albicans CAS-resistant clinical isolates (see, FIG. 4, Panel C). BRI MICs for each (. albicans CAS-resistant isolate is 20 to 80 μM (Table 6) and the combination of BRI 5 to 20 μM (0.25× MIC)+CAS 0.5 μg/ml decreased CAS-resistance of DPL1006, DPL1007, DPL1009, DPL1010, and DPL1011 7-, 10-, 4-, 10-, and 2-fold, respectively (see, FIG. 4, Panel C). BRI MIC for C. auris is 80 μM (Table 6) and BRI 10 μM+CAS 0.125 g/ml inhibited about 95% C. auris metabolic activity in clinical isolates 467/2015, 468/2015, 469/2015, 470/2015, and 474/2015 (see, FIG. 4, Panel D). In one of these clinical isolates, 467/2015, BRI 10 μM+CAS 0.5 μg/ml is able to inhibit 100% metabolic activity and survival, potentiating caspofungin activity by at least 2-fold (see, FIG. 4, Panel E).

Taken together, these results indicate that BRI is able to potentiate CAS activity for different human fungal pathogens, including C. neoformans. Interestingly, C. neoformans is very sensitive to BRI alone and BRI is fungicidal against this fungus. Thus, BRI is a novel therapeutic against C. neoformans alone or in combination with CAS since it potentiates the latter's activity into a fungicidal drug. BRI is also able to convert CAS into a fungicidal drug in C. auris.

### Example 5: BRI Combined With CAS is Not Toxic to Human Cells and Decreases the A. fumigatus Fungal Burden in a Chemotherapeutic Murine Model

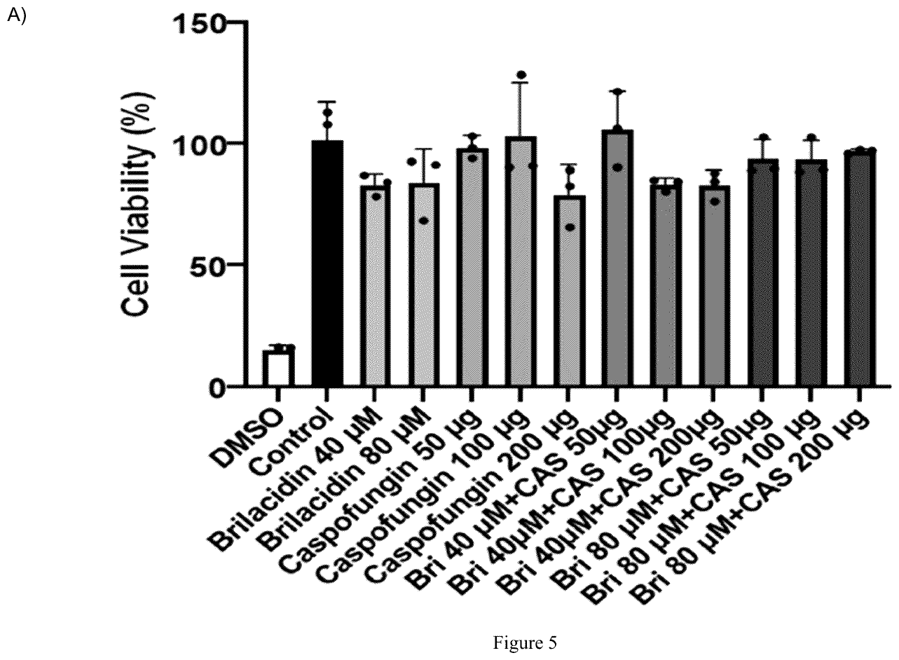

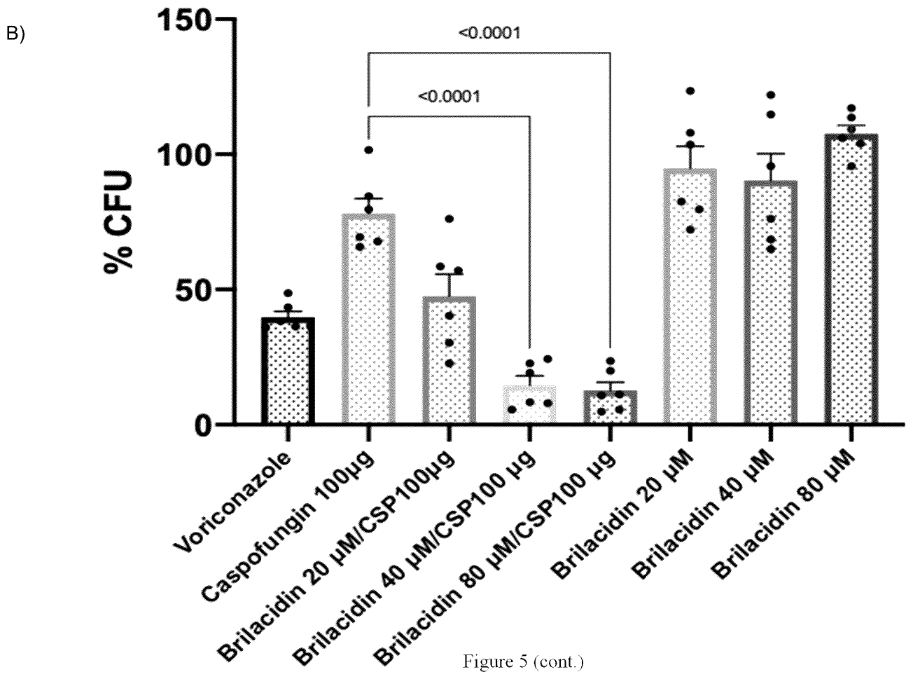

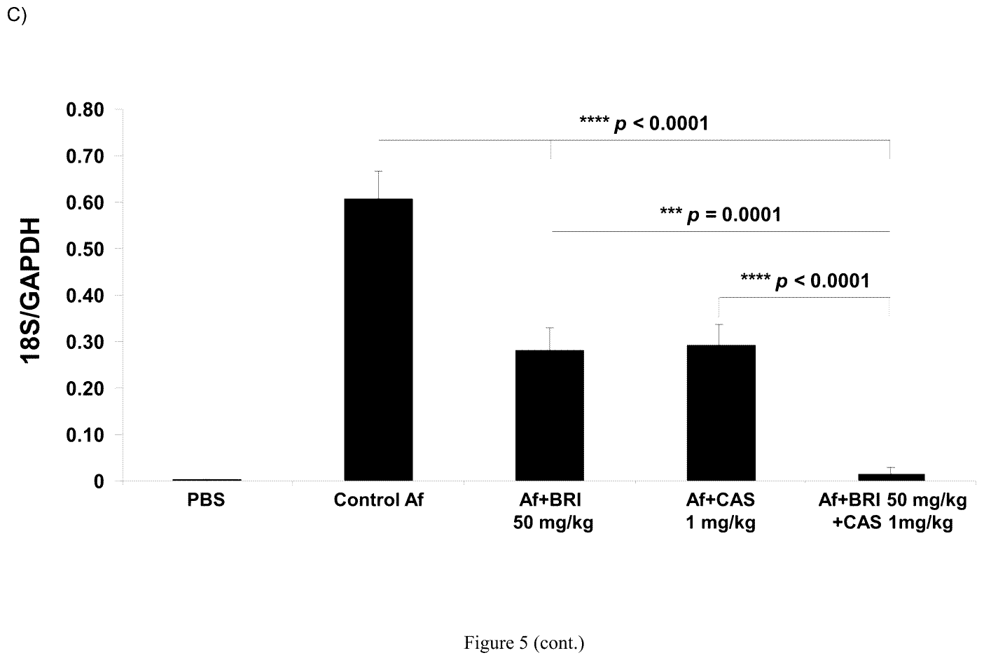

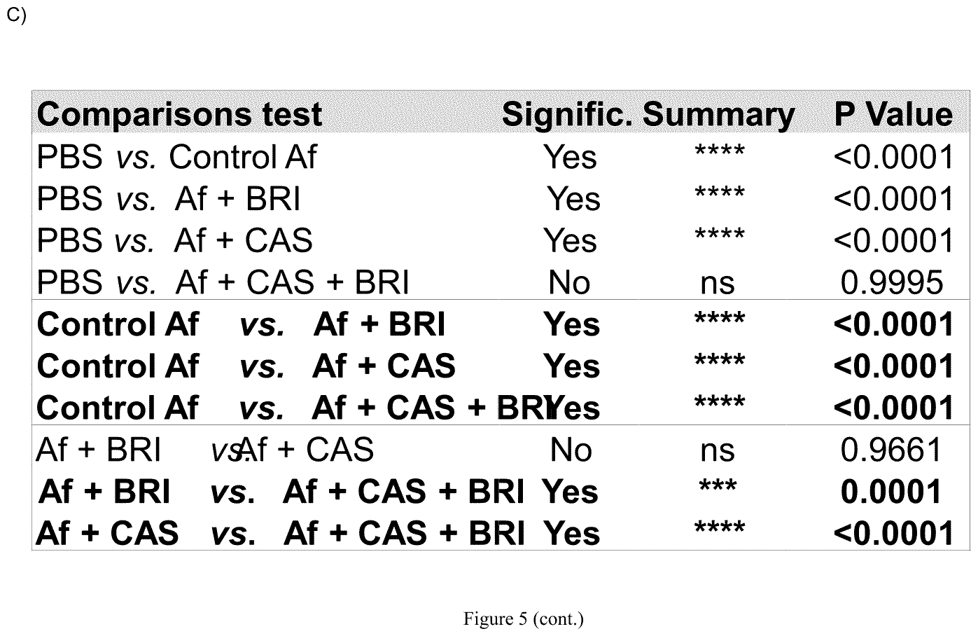

Toxicity assessment of brilacidin in A549 pulmonary cells was initially performed by incubating the cells either with 40 or 80 μM of BRI with or without increasing CAS concentrations for 48 hours, after which cell viability was assessed by XTT assay (see, FIG. 5, Panel A). As a positive control, DMSO 10% reduced cell viability by 80%. Neither BRI, CAS, or their combinations reduced cell viability when compared to the control (see, FIG. 5, Panel A). Also compared was the ability of the combination of BRI+CAS to the standard of care, VOR, in controlling A. fumigatus cell growth when infecting A549 pulmonary cells. While VOR, CAS, and BRI monotherapy killed ˜60%, 25%, and 0%-10%, respectively, a combination of BRI (20-80 μM)+CAS (100 μg/ml) resulted in 50-85% A. fumigatus killing (see. FIG. 5, Panel B).

It was also investigated whether BRI+CAS could impact A. fumigatus virulence in a chemotherapeutic murine model of IPA. Fungal burden in the lungs was approximately 50% reduced after 3 days post-infection in mice treated either with CAS (1 mg/kg) or BRI (50 mg/kg) when compared with the non-treated mice (see, FIG. 5, Panel C). However, the combination of BRI+CAS significantly reduced the fungal burden by ˜95% when compared with the non-treated mice (see, FIG. 5, Panel C). These results strongly indicate that the combination BRI+CAS is able to clear A. fumigatus infection in the lungs in a chemotherapeutic murine model of IPA.

Taken together, these data indicate that the combination treatment of BRI+CAS is non-toxic to mammalian cells in vitro and is able to enhance clearance of A. fumigatus infection in pulmonary cells in vitro and in vivo when compared to monotherapy alone.

### Example 6: BRI Inhibits Many Species of Fungi

It was investigated if BRI could inhibit the growth of a range of fungal species in vitro, with the testing including multiple fungal isolates for certain species. For each pathogen/strain, the BRI concentration resulting in (1) a prominent reduction in growth, i.e., 50% inhibition compared to the growth control (MIC₅₀), and (2) a complete inhibition of growth (100% inhibition vs. growth control, MIC₁₀₀) are presented in Table 7.

TABLE 7

| | | 50% | 100% |
|---|---|---|---|
| | | Inhibition | Inhibition |
| Pathogen | Strain | BRI (μg/ml) | BRI (μg/ml) |
| Cryptococcus | 00-288 | 1.0 | 2.0 |
| neoformans | 01-126 | 1.0 | 1.0 |
| | 06-71 | 1.0 | 1.0 |
| | 00-289 | 1.0 | 2.0 |
| | 97-370 | 2.0 | 2.0 |
| | CN9759 | 1.0 | 8.0 |
| | 17-66 | 2.0 | 2.0 |
| | 15-101 | 4.0 | 8.0 |
| Lomentospora | 15-99 | 4.0 | 8.0 |
| prolificans | 15-97 | 4.0 | 8.0 |
| | 15-98 | 4.0 | 8.0 |
| | 94-58 | 8.0 | 16 |
| | 10-03 | 4.0 | 8.0 |
| | 15-100 | 8.0 | 16 |
| Scedosporium | 12-13 | 4.0 | 8.0 |
| apiospermum complex | 98-38 | 2.0 | 8.0 |
| | 01-48 | 4.0 | 16 |
| | 10-23 | 2.0 | 4.0 |
| | 18-46 | 8.0 | 16 |
| Fusarium species | 07-144 | 4.0 | 16 |
| | 22-51 | 8.0 | 16 |
| | 07-136 | 2.0 | 16 |
| | 00-137 | 2.0 | 32 |
| | 19-171 | 2.0 | 32 |
| | 12-22 | 1.0 | 64 |
| | 22-1 | 2.0 | 32 |
| Mucorales | 16-88 | 4.0 | 16 |
| Rhizopus species | 20-235 | 16 | 32 |
| | 21-01 | 8.0 | 16 |
| | 13-91 | 2.0 | 8.0 |
| | 94-2 | 2.0 | 32 |
| | 21-85 | 4.0 | 64 |
| Mucorales | 20-177 | 16 | 32 |
| Mucor species | 15-64 | 4.0 | 64 |
| | 13-39 | 4.0 | 32 |
| | 13-127 | 4.0 | >64 |
| Mucorales | 07-140 | 2.0 | 16 |
| Unspeciated | | | |
| zygomycete | | | |
| Candida albicans | 20-132 | 1.0 | 4.0 |
| | 5 | 4.0 | >64 |
| | 21-76 | 32 | >64 |
| | (note fluconazole- | | |
| | resistant) | | |
| Candida auris | 20-253 | >64 | >64 |
| Candida krusei | 03-287 | 8.0 | 16 |
| | (note fluconazole- | | |
| | resistant) | | |
| Candida lusitaniae | 22-16 | 8.0 | 8.0 |
| Torulopsis glabrata | 22-21 | 64 | >64 |
| Acremonium species | 18-51 | 4 | >64 |
| Exserohilum species | 19-48 | 1.0 | 16 |
| Aspergillus niger | 22-4 | 8 | 16 |
| Aspergillus lentulus | 14-39 | 32 | >64 |
| Aspergillus terreus | 12-70 | >64 | >64 |
| Aspergillus fumigatus | 18-31 | >64 | >64 |
| | 13-130 | >64 | >64 |
| | 19-12 | >64 | >64 |
| | 21-23 | 64 | >64 |
| | 09-03 | >64 | >64 |
| | 18-32 | 64 | >64 |
| | 18-117 | >64 | >64 |
| | 13-30 | >64 | >64 |
| | 11-13 | >64 | >64 |
| | 09-117 | >64 | >64 |
| | 10AF | 64 | >64 |

Potent inhibitory activity of BRI was noted for many species of fungi. Particularly noteworthy are the results for Cryptococcus (currently a cause of almost a million cases globally annually), Mucorales (for which conventional therapy results are poor), and other lethal pathogens for which current therapy is poor or lacking (Fusarium, Scedosporium, Lomentospora, Acremonium, Exserohilum).

### Example 7: BRI Does Not Interfere with the Action of Conventional Antifungals, and Sometimes Potentiates Them

The in vitro interaction of BRI in combination with conventional antifungals was investigated in a number of critical fungal pathogens. Results are show in Table 8 by drug pairing, for each drug alone (MIC values) and for the combination of BRI+test antifungal (FIC index values).

TABLE 8

| | | | | FICi | FICi | |
|---|---|---|---|---|---|---|
| Isolate/ | | MIC₅₀ | MIC₁₀₀ | for 50% | for 100% | |
| Strain | Drug | (μg/ml) | (μg/ml) | inhibition | inhibition | Comment |
| C. | Fluconazole | 1 | 1 | — | — | |
| neoformans | (FLU) | | | | | |
| CN9759 | BRI | 1 | 8 | — | — | |
| | FLU + BRI | — | — | 0.75 | 0.25 | |
| Mucor sp. | Posaconazole | 1 | >16 | — | — | |
| 13-127 | (POSA) | | | | | |
| | BRI | 4 | 64 | — | — | |
| | POSA + BRI | — | — | 0.625 | Indifference | |
| Mucor sp. | Amphotericin | 0.25 | 0.5 | — | — | |
| 13-127 | B (AMPB) | | | | | |
| | BRI | 4 | 64 | — | — | |
| | AMPB + BRI | — | — | Indifference | 0.75 | |
| Mucor sp. | Micafungin | >16 | >16 | — | — | |
| 13-127 | (MICA) | | | | | |
| | BRI | 4 | 64 | — | — | |
| | MICA + BRI | — | — | Indifference | 0.53 | |
| C. | Micafungin | ≤0.03 | 0.25 | — | — | |
| albicans | (MICA) | | | | | |
| 5 | BRI | 4 | >64 | — | — | |
| | MICA + BRI | — | — | Indifference | Indifference | |
| A. | Flucytosine | >64 | >64 | — | — | |
| fumigatus | (FLCY) | | | | | |
| 10AF | BRI | 64 | >64 | — | — | |
| | FLCY + BRI | — | — | Indifference | Indifference | |
| A. | Voriconazole | 0.125 | 1 | — | — | |
| fumigatus | (VOR) | | | | | |
| 10AF | BRI | 64 | >64 | — | — | |
| | VOR + BRI | — | — | Indifference | Antagonism | There is a |
| | | | | | | trend toward |
| | | | | | | antagonism |
| | | | | | | at 50% also |
| A. | Micafungin | ≤0.06 | >16 | — | — | |
| fumigatus | (MICA) | | | | | |
| 21-23 | BRI | 64 | >64 | — | — | |
| | MICA + BRI | — | — | Indifference | Indifference | There is a |
| | | | | | | trend toward |
| | | | | | | synergy at |
| | | | | | | both 50% |
| | | | | | | and 100% |

BRI = brilacidin; MIC50 = lowest drug concentration that caused a 50% decrease in growth with respect to the untreated control; MIC100 = lowest drug concentration without growth; FICi = Fractional Inhibitory Concentration index. Drug interactions were classified as strong synergism when FICi < 0.5; weak synergism when 0.5-<1; additive when 1-<2; indifferent when FICI = 2; and antagonistic when FICi > 2. “Indifference” in this context means each of the drugs acts independently of the other, each is fully active in the presence of the other; there is no antagonism nor synergy. “A trend towards” means there is some interaction between the drugs, but the interaction does not meet stringent cutoffs of 50% or 100% endpoints, whichever of those is referred to.

BRI alone had favorable in vitro activity against the organisms tested here, with the exception of Aspergillus. Looking across the various species and drugs tested, the most common interaction of BRI with other antifungals is “Indifference”. There was only 1 instance of antagonism. There were 4 instances of weak synergy, and one of strong synergy.

These results indicate that, in general, brilacidin does not interfere with the action of other antifungals, and sometimes potentiates them.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A pharmaceutical composition comprising a compound having the formula: 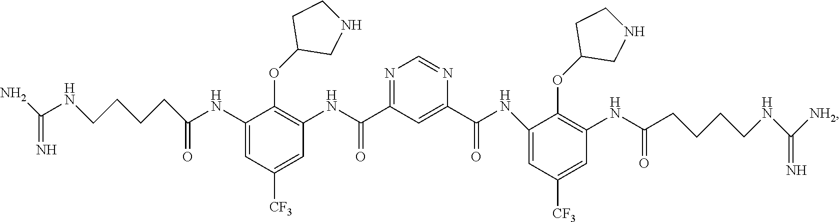 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3OC3CCNC3)ncn2)c1OC1CCNC1]
or a pharmaceutically acceptable salt thereof, and one or more other anti-fungal agents which is an azole or an echinocandin.

2. The method of claim 1, wherein the compound has the formula: 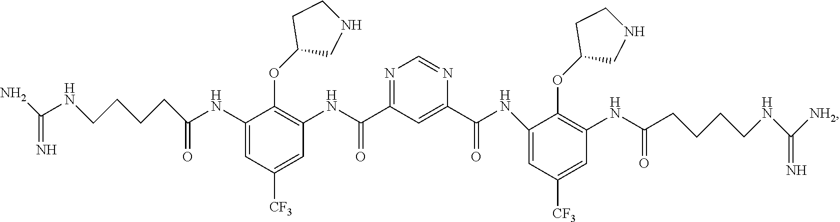 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3O[C@@H]3CCNC3)ncn2)c1O[C@@H]1CCNC1]
or a pharmaceutically acceptable salt thereof.

3. The method of claim 1 or claim 2, wherein the azole is itraconazole, posaconazole, voriconazole (VOR), or isavuconazole.

4. The method of claim 3, wherein the azole is VOR.

5. The method of claim 1 or claim 2, wherein the echinocandin is caspofungin (CAS).

6. A method of treating or preventing a Cryptococcus fungal infection in a mammal comprising administering to the mammal in need thereof a compound having the formula: 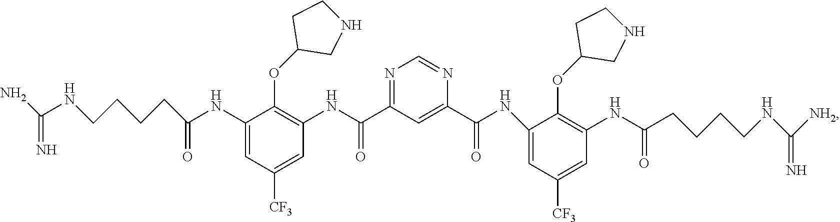 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3OC3CCNC3)ncn2)c1OC1CCNC1]
or a pharmaceutically acceptable salt thereof.

7. The method of claim 6, wherein the compound has the formula: 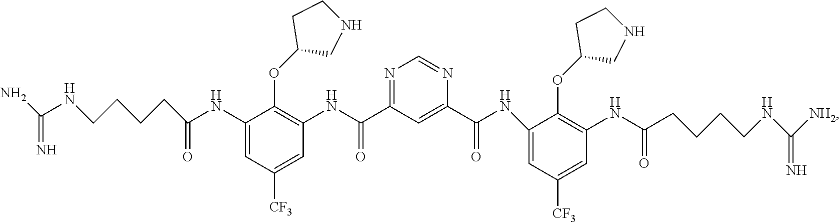 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3O[C@@H]3CCNC3)ncn2)c1O[C@@H]1CCNC1]
or a pharmaceutically acceptable salt thereof.

8. The method of claim 6 or claim 7, wherein the Cryptococcus fungal infection comprises Cryptococcus neoformas.

9. A method of killing or inhibiting the growth of a Cryptococcus species comprising contacting the Cryptococcus species with a compound having the formula: 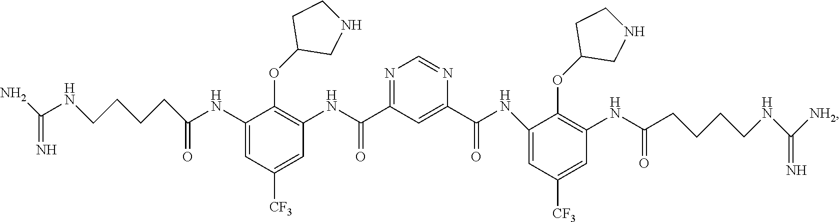 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3OC3CCNC3)ncn2)c1OC1CCNC1]
or a pharmaceutically acceptable salt thereof.

10. The method of claim 9, wherein the compound has the formula: 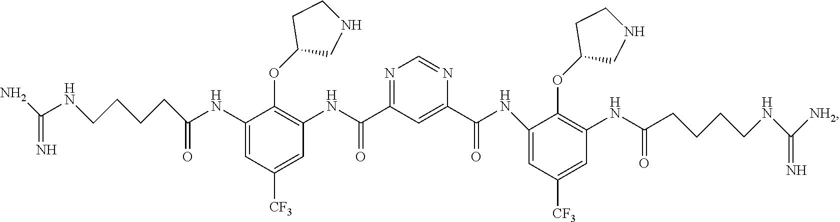 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3O[C@@H]3CCNC3)ncn2)c1O[C@@H]1CCNC1]
or a pharmaceutically acceptable salt thereof.

11. The method of claim 9 or claim 10, wherein the Cryptococcus species is Cryptococcus neoformas.

12. A method of treating or preventing a fungal infection in a mammal comprising administering to the mammal in need thereof:
a compound having the formula: 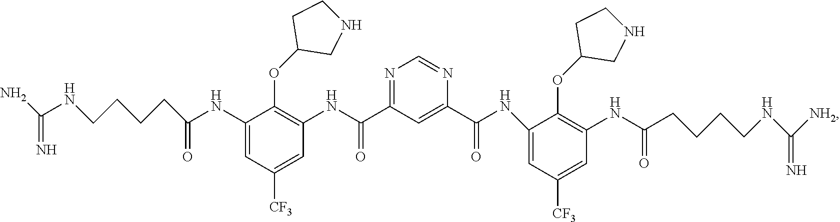 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3OC3CCNC3)ncn2)c1OC1CCNC1]
or a pharmaceutically acceptable salt thereof; and
One or more other anti-fungal agents which is an azole or an echinocandin.

13. The method of claim 12, wherein the compound has the formula: 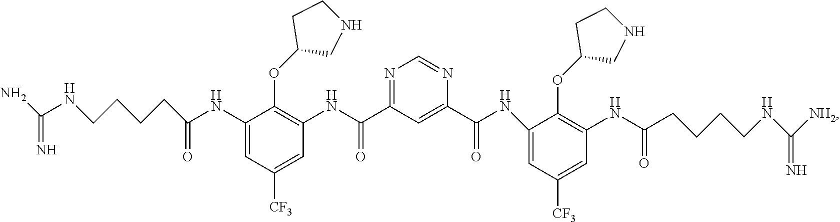 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3O[C@@H]3CCNC3)ncn2)c1O[C@@H]1CCNC1]
or a pharmaceutically acceptable salt thereof.

14. The method of claim 12 or claim 13, wherein the azole is itraconazole, posaconazole, voriconazole (VOR), or isavuconazole.

15. The method of claim 14, wherein the azole is VOR.

16. The method of claim 12 or claim 13, wherein the echinocandin is caspofungin (CAS).

17. The method of any one of claims 12 to 16, wherein the fungal infection is an Aspergillus spp., Fusarium spp., Malessezia spp., Candida spp., or Cryptococcus spp. infection.

18. The method of any one of claims 12 to 16, wherein the fungal infection is an Aspergillus spp. infection.

19. The method of claim 18, wherein the Aspergillus spp. infection is an Aspergillus fumigatus infection.

20. The method of claim 18, wherein the Aspergillus spp. infection is an Aspergillus favus infection.

21. The method of claim 18, wherein the Aspergillus spp. infection is an Aspergillus niger infection.

22. The method of claim 18, wherein the Aspergillus spp. infection is an Aspergillus terreus infection.

23. The method of any one of claims 12 to 16, wherein the fungal infection is an Fusarium spp. infection.

24. The method of claim 23, wherein the Fusarium spp. infection is a Fusarium solani infection.

25. The method of claim 23, wherein the Fusarium spp. infection is a Fusarium moniliforme infection.

26. The method of claim 23, wherein the Fusarium spp. infection is a Fusarium proliferartum infection.

27. The method of any one of claims 12 to 16, wherein the fungal infection is an Malessezia spp. infection.

28. The method of claim 27, wherein the Malessezia spp. infection is a Malessezia pachydermatis infection.

29. The method of any one of claims 12 to 16, wherein the fungal infection is a Candida spp. infection.

30. The method of claim 29, wherein the Candida spp. infection is a Candida albicans infection.

31. The method of claim 29, wherein the Candida spp. infection is a Candida glabrata infection.

32. The method of claim 29, wherein the Candida spp. infection is a Candida tropicalis infection. 33 The method of claim 29, wherein the Candida spp. infection is a Candida krusei infection.

34. The method of claim 29, wherein the Candida spp. infection is a Candida auris infection.

35. The method of any one of claims 12 to 16, wherein the fungal infection is a Cryptococcus spp. infection.

36. The method of claim 35, wherein the Cryptococcus spp. infection is a Cryptococcus neoformans infection.

37. The method of any one of claims 12 to 16, wherein the fungal infection is a Chrysosporium parvum, Metarhizium anisopliae, Phaeoisaria clematidis, or Sarcopodium oculorum infection.

38. The method of any one of claims 12 to 16, wherein the fungal infection is a Mucorales infection.

39. The method of claim 38, wherein the Mucorales infection is a Mucor spp., Rhizopus spp., Lichtheimia spp., or Rhizomucor spp. infection.

40. The method of claim 39, wherein the Mucor spp. infection is a M. circinelloides infection.

41. The method of claim 39, wherein the Rhizopus spp. infection is a Rhizopus delemar infection or a Rhizopus oryzae infection.

42. The method of claim 39, wherein the Lichtheimia spp. infection is a Lichtheimia corymbifera infection.

43. A method of killing or inhibiting the growth of a fungus comprising contacting the fungus with:
a compound having the formula: 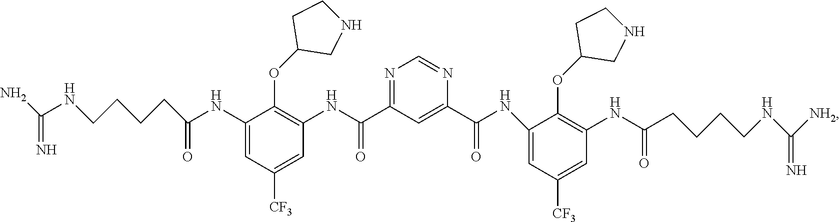 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3OC3CCNC3)ncn2)c1OC1CCNC1]
or a pharmaceutically acceptable salt thereof; and
One or more other anti-fungal agents which is an azole or an echinocandin.

44. The method of claim 43, wherein the compound has the formula: 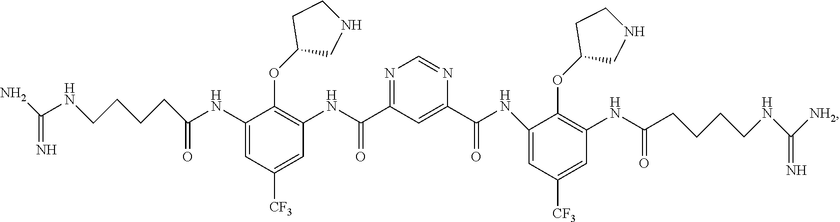 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3O[C@@H]3CCNC3)ncn2)c1O[C@@H]1CCNC1]
or a pharmaceutically acceptable salt thereof.

45. The method of claim 43 or claim 44, wherein the azole is itraconazole, posaconazole, voriconazole (VOR), or isavuconazole.

46. The method of claim 45, wherein the azole is VOR.

47. The method of claim 43 or claim 44, wherein the echinocandin is caspofungin (CAS).

48. The method of any one of claims 43 to 47, wherein the fungal infection is an Aspergillus spp., Fusarium spp., Malessezia spp., Candida spp., or Cryptococcus spp. infection.

49. The method of any one of claims 43 to 47, wherein the fungal infection is an Aspergillus spp. infection.

50. The method of claim 49, wherein the Aspergillus spp. infection is an Aspergillus fumigatus infection.

51. The method of claim 49, wherein the Aspergillus spp. infection is an Aspergillus favus infection.

52. The method of claim 49, wherein the Aspergillus spp. infection is an Aspergillus niger infection.

53. The method of claim 49, wherein the Aspergillus spp. infection is an Aspergillus terreus infection.

54. The method of any one of claims 43 to 47, wherein the fungal infection is an Fusarium spp. infection.

55. The method of claim 54, wherein the Fusarium spp. infection is a Fusarium solani infection.

56. The method of claim 54, wherein the Fusarium spp. infection is a Fusarium moniliforme infection.

57. The method of claim 54, wherein the Fusarium spp. infection is a Fusarium proliferartum infection.

58. The method of any one of claims 43 to 47, wherein the fungal infection is an Malessezia spp. infection.

59. The method of claim 58, wherein the Malessezia spp. infection is a Malessezia pachydermatis infection.

60. The method of any one of claims 43 to 47, wherein the fungal infection is a Candida spp. infection.

61. The method of claim 60, wherein the Candida spp. infection is a Candida albicans infection.

62. The method of claim 60, wherein the Candida spp. infection is a Candida glabrata infection.

63. The method of claim 60, wherein the Candida spp. infection is a Candida tropicalis infection.

64. The method of claim 60, wherein the Candida spp. infection is a Candida krusei infection.

65. The method of claim 60, wherein the Candida spp. infection is a Candida auris infection.

66. The method of any one of claims 43 to 47, wherein the fungal infection is a Cryptococcus spp. infection.

67. The method of claim 66, wherein the Cryptococcus spp. infection is a Cryptococcus neoformans infection.

68. The method of any one of claims 43 to 47, wherein the fungal infection is a Chrysosporium parvum, Metarhizium anisopliae, Phaeoisaria clematidis, or Sarcopodium oculorum infection.

69. The method of any one of claims 43 to 47, wherein the fungal infection is a Mucorales infection.

70. The method of claim 69, wherein the Mucorales infection is a Mucor spp., Rhizopus spp., Lichtheimia spp., or Rhizomucor spp. infection.

71. The method of claim 70, wherein the Mucor spp. infection is a M. circinelloides infection.

72. The method of claim 70, wherein the Rhizopus spp. infection is a Rhizopus delemar infection or a Rhizopus oryzae infection.

73. The method of claim 70, wherein the Lichtheimia spp. infection is a Lichtheimia corymbifera infection.
